(19) United States
(12) Patent Application Publication
(10) Pub. No.: US 2024/0392027 A1
(43) Pub. Date: Nov. 28, 2024

(54) COMPOSITIONS COMPRISING A BISPECIFIC GPRC5D/CD3 ANTIBODY

(71) Applicant: Janssen Biotech Inc., Horsham, PA (US)

(72) Inventors: Kevin B. Vargo, Collegeville, PA (US); Daniel P. Heinaman, Norristown, PA (US); Curie K Javiya, Harleysville, PA (US); Salman Muzammil, Furlong, PA (US)

(21) Appl. No.: 18/588,224

(22) Filed: Feb. 27, 2024

Related U.S. Application Data
(60) Provisional application No. 63/487,514, filed on Feb. 28, 2023.
(60) Provisional application No. 63/588,488, filed on Oct. 6, 2023.

(51) Int. Cl. C07K 16/28; A61K 39/00; A61P 35/00

(52) U.S. Cl. CPC C07K 16/2896; A61P 35/00; C07K 16/2809; A61K 2039/505; A61K 2039/54; C07K 2317/24; C07K 2317/31; C07K 2317/732; C07K 2317/94

(57) ABSTRACT

Provided herein are aqueous pharmaceutical compositions comprising formulations of a bispecific GPRC5D/CD3 antibody or an antigen-binding fragment thereof and methods of preparing the same. Also provided herein are methods of treating cancer in a subject in need thereof by administering to the subject the aqueous pharmaceutical compositions as disclosed herein. Further provided herein are kits and articles of manufacture comprising the aqueous pharmaceutical compositions as disclosed herein.

### CROSS-REFERENCE TO RELATED APPLICATIONS

This application claims priority to U.S. Provisional Application Ser. No. 63/487,514, filed Feb. 28, 2023, and 63/588,488, filed Oct. 6, 2023, the entire contents of which are incorporated herein by reference in their entirety.

### REFERENCE TO SEQUENCE LISTING SUBMITTED ELECTRONICALLY

This application contains a sequence listing, which is submitted electronically in XML format and is hereby incorporated by reference in its entirety. Said XML copy, created on Oct. 6, 2023, is named “258199.061201 (JBI6770USNP1) Sequence Listing.xml” and is 36,888 bytes in size.

### FIELD OF THE INVENTION

Disclosed are compositions and methods for formulating a pharmaceutical composition comprising bispecific GPRC5D/CD3 antibodies.

### BACKGROUND OF THE INVENTION

Multiple myeloma (MM) is a cancer of the plasma cells. Mechanistically, multiple myeloma is characterized by production of monoclonal proteins (M-proteins) comprised of pathological immunoglobulins or fragments of such, which have lost their function. The proliferation of multiple myeloma cells leads to subsequent displacement from the normal bone marrow niche, while overproduction of M-proteins causes characteristic osteolytic lesions, increased susceptibility to infections, hypercalcemia, renal insufficiency or failure, and neurological complications.

Treatment options for multiple myeloma have improved over time and vary depending on the aggressiveness of the disease, underlying prognostic factors, physical condition of the patient, and existing comorbidities. Therapeutic options include proteasome inhibitors (PIs), immunomodulatory drugs (IMiDs), monoclonal antibodies (mAbs), and stem cell transplantation.

Despite these therapeutic achievements, the disease recurs and is associated with additional risk factors (e.g., comorbidities or increasing age), thus warranting the need for novel therapeutic approaches, such as new dosage and treatment regimens. In particular, in the elderly population, for which stem cell transplantation is often not a viable option, and in patients with refractory disease who exhausted all available therapies, multiple myeloma remains an incurable malignancy and an unmet medical need with significant morbidity and mortality.

T cell redirected killing is a desirable mode of action in many therapeutic areas. In general, T cell redirecting molecules are engineered to have at least two antigen binding sites wherein one site binds a surface antigen on a target cell and the other site binds a T cell surface antigen. There remains a need in the art for pharmaceutical compositions comprising such antibodies for treatment of patients with multiple myeloma, including compositions that are stable for long periods of time within a range of temperatures.

### SUMMARY OF THE INVENTION

Disclosed herein are aqueous pharmaceutical compositions comprising specific formulations of a G-protein coupled receptor, class C, group 5, member D (GPRC5D)/cluster of differentiation 3 (CD3) antibody. In certain embodiments, the antibody is Talquetamab (Tal), an investigational bispecific antibody that binds to G Protein-Coupled Receptor Class C Group 5 Member D (GPRC5D) and CD3.

In some, provided herein are aqueous pharmaceutical compositions comprising:
(a) a concentration of about 0.5 mg/mL to about 3.5 mg/mL of a bispecific G Protein-Coupled Receptor Class C Group 5 Member D (GPRC5D)/cluster of differentiation 3 (CD3) antibody or antigen-binding fragment thereof, the bispecific GPRC5D/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
(b) about 10 mM to about 20 mM of acetate and/or pharmaceutically acceptable acetate salt;
(c) about 6% (w/v) to about 10% (w/v) of sucrose;
(d) about 7 μg/mL to about 33 μg/mL of ethylenediaminetetraacetic acid (EDTA);
(e) about 0.01% to about 0.07% polysorbate 20; and
(f) a pH from about 4.6 to about 5.8.

In some embodiments, provided herein are aqueous pharmaceutical compositions comprising:
(a) a concentration of about 25 mg/mL to about 55 mg/mL of a bispecific G Protein-Coupled Receptor Class C Group 5 Member D (GPRC5D)/cluster of differentiation 3 (CD3) antibody or antigen-binding fragment thereof, the bispecific GPRC5D/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
(b) about 10 mM to about 20 mM of acetate and/or pharmaceutically acceptable acetate salt;
(c) about 6% (w/v) to about 10% (w/v) of sucrose;
(d) about 7 μg/mL to about 33 μg/mL of ethylenediaminetetraacetic acid (EDTA);
(e) about 0.01% to about 0.07% polysorbate 20; and
(f) a pH from about 4.6 to about 5.8.

In some embodiments, the aqueous pharmaceutical compositions comprise 2 mg/mL of the bispecific GPRC5D/CD3 antibody, 15 mM of acetate and/or pharmaceutically acceptable acetate salt, 8% (w/v) sucrose, 20 μg/mL of EDTA, 0.04% PS 20, and a pH of 5.2.

In some embodiments, the aqueous pharmaceutical compositions comprise 40 mg/mL of the bispecific GPRC5D/CD3 antibody, 15 mM of acetate and/or pharmaceutically acceptable acetate salt, 8% (w/v) sucrose, 20 μg/mL of EDTA, 0.04% PS 20, and a pH of 5.2.

In some embodiments, a method for treating cancer in a subject in need thereof is provided. In some embodiments, the method comprises administering to the subject the aqueous pharmaceutical composition as provided for herein.

In some embodiments, a method for preparing aqueous compositions of the bispecific G Protein-Coupled Receptor Class C Group 5 Member D (GPRC5D)/cluster of differentiation 3 (CD3) antibody or antigen-binding fragment thereof are provided. In some embodiments, the bispecific GPRC5D/CD3 antibody or antigen-binding fragment thereof comprises:
(1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
   In some embodiments, the methods comprises combining a composition comprising about 2 mg/mL of the bispecific GPRC5D/CD3 antibody, about 15 mM of acetate and/or a pharmaceutically acceptable acetate salt, about 8% (w/v) sucrose, about 18 μg/mL of EDTA, and about 0.04% polysorbate (PS) 20, wherein the stable aqueous pharmaceutical composition has a pH of about 5.2.

In some embodiments, a method for preparing aqueous compositions of the bispecific G Protein-Coupled Receptor Class C Group 5 Member D (GPRC5D)/cluster of differentiation 3 (CD3) antibody or antigen-binding fragment thereof are provided. In some embodiments, the bispecific GPRC5D/CD3 antibody or antigen-binding fragment thereof comprises:
(5) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
(6) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(7) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
(8) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
   In some embodiments, the methods comprises combining a composition comprising about 40 mg/mL of the bispecific GPRC5D/CD3 antibody, about 15 mM of acetate and/or a pharmaceutically acceptable acetate salt, about 8% (w/v) sucrose, about 18 μg/mL of EDTA, and about 0.04% polysorbate (PS) 20, wherein the stable aqueous pharmaceutical composition has a pH of about 5.2.

In some embodiments, the aqueous pharmaceutical compositions are stable. In some embodiments, stability is defined as provided for herein.

In some embodiments, the GPRC5D/CD3 antibody is talquetamab.

In some embodiments, kits comprising the pharmaceutical aqueous compositions, as provided for herein, and instructions for use thereof, are provided.

In some embodiments, an article of manufacture is provided. In some embodiments, the article of manufacture comprises a container holding the aqueous pharmaceutical composition as provided for herein.

In some embodiments, use of the aqueous pharmaceutical composition as provided for herein for treating a cancer in a subject are provided. In some embodiments, the use comprises administering the aqueous pharmaceutical composition to the subject in need thereof.

### BRIEF DESCRIPTION OF FIGURES

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 illustrates main effects of test formulation parameters vs. % monomer measured by SEC.

FIG. 2 illustrates main effects of test formulation parameters vs. % aggregate measured by SEC.

FIG. 3 illustrates main effects of test formulation parameters vs. % fragment measured by SEC.

FIG. 4 illustrates main effects of acetate test formulation parameters vs. % monomer measured by SEC.

FIG. 5 illustrates main effects of acetate test formulation parameters vs. % aggregate measured by SEC.

FIG. 6 illustrates main effects of acetate test formulation parameters vs. % fragment measured by SEC.

### DETAILED DESCRIPTION OF THE INVENTION

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

The term “comprising” is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The term “antibody,” and like terms is meant in a broad sense and includes immunoglobulin molecules or fragments thereof, including monoclonal antibodies (such as murine, human, human-adapted, humanized, and chimeric monoclonal antibodies), antibody fragments, bispecific or multispecific antibodies, dimeric, tetrameric or multimeric antibodies, and single chain antibodies.

Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Antibody fragment” refers to a portion of an immunoglobulin molecule that retains the antigen binding properties of the parental full-length antibody. Exemplary antibody fragments are heavy chain complementarity determining regions (HCDR) 1, 2, and 3, light chain complementarity determining regions (LCDR) 1, 2, and 3, a heavy chain variable region (VH), or a light chain variable region (VL). Antibody fragments include: a Fab fragment, a monovalent fragment consisting of the VL, VH, constant light (CL), and constant heavy 1 (CH1) domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; and a domain antibody (dAb) fragment, which consists of a VH domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in Int'l Pat. Pub. Nos. WO1998/44001, WO1988/01649, WO1994/13804, and WO1992/01047. These antibody fragments are obtained using techniques well known to those of skill in the art, and the fragments are screened for utility in the same manner as are full length antibodies.

An antibody variable region consists of a “framework” region interrupted by three “antigen binding sites.” The antigen binding sites are defined using various terms: (i) Complementarity Determining Regions (CDRs), three in the VH (HCDR1, HCDR2, HCDR3), and three in the VL (LCDR1, LCDR2, LCDR3) are based on sequence variability (Wu and Kabat J Exp Med 132:211-50, 1970; Kabat et al. Sequences of Proteins of Immunological Interest, 5th Ed. Public Health Service, National Institutes of Health, Bethesda, Md., 1991); and (ii) “Hypervariable regions” (“HVR” or “HV”), three in the VH (H1, H2, H3) and three in the VL (L1, L2, L3) refer to the regions of the antibody variable domains which are hypervariable in structure as defined by Chothia and Lesk (Chothia and Lesk Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro Mol Recognit 17:132-43, 2004). The International ImMunoGeneTics (IMGT) database (www_imgt_org) provides a standardized numbering and definition of antigen-binding sites. The correspondence between CDRs, HVs and IMGT delineations is described in Lefranc et al., Dev Comparat Immunol 27:55-77, 2003.

“Monoclonal antibody” refers to a preparation of antibody molecules of a single molecular composition. A monoclonal antibody composition displays a single binding specificity and affinity for a particular epitope, or in a case of a bispecific monoclonal antibody, a dual binding specificity to two distinct epitopes. Monoclonal antibody therefore refers to an antibody population with single amino acid composition in each heavy and each light chain, except for possible well-known alterations such as removal of C-terminal lysine from the antibody heavy chain. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Bispecific” refers to an antibody that specifically binds two distinct antigens or two distinct epitopes within the same antigen. The bispecific antibody can have cross-reactivity to other related antigens, for example to the same antigen from other species (homologs), such as human or monkey, for example Macaca cynomolgus (cynomolgus, cyno) or Pan troglodytes, or can bind an epitope that is shared between two or more distinct antigens.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” can contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody can include substitutions in the frameworks so that the frameworks cannot be exact copies of expressed human immunoglobulin or human immunoglobulin germline gene sequences.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“GPRC5D×CD3 bispecific antibody” refers to a molecule containing two or more binding regions, wherein one of the binding regions specifically binds the cell surface antigen G Protein-Coupled Receptor Class C Group 5 Member D antigen (GPRC5D) on a target cell or tissue and wherein a second binding region of the molecule specifically binds a T cell antigen CD3. This dual/multi-target binding ability recruit T cells to the target cell or tissue leading to the eradication of the target cell or tissue. GPRC5D/CD3 bispecific antibodies are described in U.S. Pat. No. 10,562,968, which is incorporated by reference herein in its entirety.

“GPRC5D” refers to human G-protein coupled receptor family C group 5 member D having the amino acid sequence shown in SEQ ID NO: 21.

(SEQ ID NO: 21)
MYKDCIESTGDYFLLCDAEGPWGIILESLAILGIVVTILLLLAFLFLMR
KIQDCSQWNVLPTQLLFLLSVLGLFGLAFAFIIELNQQTAPVRYFLFGV
LFALCESCLLAHASNLVKLVRGCVSFSWTTILCIAIGCSLLQIIIATEY
VTLIMTRGMMFVNMTPCQLNVDFVVLLVYVLFLMALTFFVSKATFCGPC
ENWKQHGRLIFITVLFSIIIWVVWISMLLRGNPQFQRQPQWDDPVVCIA
LVTNAWVELLLYIVPELCILYRSCRQECPLQGNACPVTAYQHSFQVENQ
ELSRARDSDGAEEDVALTSYGTPIQPQTVDPTQECFIPQAKLSPQQDAG
GV

“CD3” refers to a human antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO: 22. SEQ ID NO: 23 shows the extracellular domain of CD3 epsilon.

(SEQ ID NO: 22)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI
(SEQ ID NO: 23)
DGNEEMGGITQTPYKVSISGTTVILTCPQYPGSEILWQHNDKNIGGDED
DKNIGSDEDHLSLKEFSELEQSGYYVCYPRGSKPEDANFYLYLRARVCE
NCMEMD

“Epitope” refers to a portion of an antigen to which an antibody specifically binds. Epitopes usually consist of chemically active (such as polar, non-polar, or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Variant” refers to a polypeptide or a polynucleotide that differs from a reference polypeptide or a reference polynucleotide by one or more modifications for example, substitutions, insertions, or deletions.

“In combination with” means that two or more therapeutics can be administered to a subject together in a mixture, concurrently as single agents, or sequentially as single agents in any order.

“Treat,” “treatment,” and like terms refer to both therapeutic treatment and prophylactic or preventative measures, and includes reducing the severity and/or frequency of symptoms, eliminating symptoms and/or the underlying cause of the symptoms, reducing the frequency or likelihood of symptoms and/or their underlying cause, improving or remediating damage caused, directly or indirectly, by the malignancy. Treatment also includes prolonging survival as compared to the expected survival of a subject not receiving treatment. Subjects to be treated include those that have the condition or disorder as well as those prone to have the condition or disorder or those in which the condition or disorder is to be prevented.

“Therapeutically effective amount” refers to an amount of the disclosed composition, which is therapeutically effective at dosages and for periods of time necessary, to achieve a desired treatment. A therapeutically effective amount may vary according to factors such as the disease state, age, sex, and weight of the subject, and the ability of the combination therapy to elicit a desired response in the subject. Exemplary indicators of a therapeutically effect amount include, for example, improved well-being of the patient, reduction of a tumor burden, arrested or slowed growth of a tumor, and/or absence of metastasis of cancer cells to other locations in the body.

“Pharmaceutical composition” refers to composition that comprises an active ingredient and a pharmaceutically acceptable carrier. As used herein, “drug product” or “DP” is synonymous with “pharmaceutical composition” or “aqueous pharmaceutical composition” unless explicitly stated otherwise. Accordingly, embodiments which refer to a “pharmaceutical composition” or an “aqueous pharmaceutical composition” or described characteristics of the same are understood to encompass and apply to a “drug product” or “DP”. Likewise, embodiments or characteristics of a “drug product” or “DP” are understood to apply encompass and apply to a “pharmaceutical composition” or an “aqueous pharmaceutical composition”

The pharmaceutical compositions, aqueous pharmaceutical compositions, drug products, or DPs of the present disclosure may be “stable”. As used herein, a pharmaceutical composition, aqueous pharmaceutical composition, drug product, or DP (all of which are synonymous) is considered stable if it adheres to the criteria as provided for herein. For example, a pharmaceutical composition, aqueous pharmaceutical composition, drug product, or DP is considered stable if it meets certain pre-determined thresholds for color of solution, pH, turbidity, number of sub-visible particles, percentage of purity (via cSDS), percentage of new peaks (via cSDS), percentage of main component (via SE-HPLC), percentage of low molecular weight species (via SE-HPLC), percentage of high molecular weight species (via SE-HPLC), percentage of main peak (via IEX), percentage of sum of the acidic peaks (via IEX), percentage of sum of the basic peaks (via IEX), protein concentration, percentage of T cell activation, percentage of PS20 (w/v), or any combination thereof. The stability of the pharmaceutical composition, aqueous pharmaceutical composition, drug product, or DP may depend on any one of the above recited criteria, or any combination thereof. Accordingly, a pharmaceutical composition, aqueous pharmaceutical composition, drug product, or DP meeting certain criteria may be considered stable even if other criteria are not met. Further, it is to be understood that the techniques of cSDS, SE-HPLC, and IEX are exemplary only. The percentage of purity, percentage of new peaks, percentage of main component, percentage of low molecular weight species, percentage of high molecular weight species, percentage of main peak, percentage of sum of the acidic peaks, and percentage of sum of the basic peaks may be determined by any known method in the art. Such methods are within the scope of the present disclosure.

“Pharmaceutically acceptable carrier” or “excipient” refers to an ingredient in a pharmaceutical composition, other than the active ingredient, which is nontoxic to a subject.

The term “cancer” as used herein is defined as disease characterized by the rapid and uncontrolled growth of aberrant cells. Cancer cells can spread locally or through the bloodstream and lymphatic system to other parts of the body. In certain embodiments, the cancer is a hematological malignancy or a solid tumor. In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation can arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

“T cell redirecting therapeutic” refers to a molecule containing two or more binding regions, wherein one of the binding regions specifically binds a cell surface antigen on a target cell or tissue and wherein a second binding region of the molecule specifically binds a T cell antigen. Examples of cell surface antigen include a tumor associated antigen, such as BCMA or GPRC5D. Examples of T cell antigen include, e.g., CD3. This dual/multi-target binding ability recruits T cells to the target cell or tissue leading to the eradication of the target cell or tissue.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

### DESCRIPTION

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated. Antibody constant chain numbering can be found for example at ImMunoGeneTics website, at IMGT Web resources at IMGT Scientific charts.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### GPRC5D/CD3 Bispecific Antibodies and Uses Thereof

Disclosed herein are aqueous pharmaceutical compositions comprising a bispecific GPRC5D/CD3 antibody, and methods of using the same for treatment of multiple myeloma, including treatment of human subjects that are relapsed or refractory to treatment with one or more prior therapies.

In some embodiments, provided herein are aqueous pharmaceutical compositions comprising:
(a) a concentration of about 0.5 mg/mL to about 100 mg/mL of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
(b) about 10 mM to about 20 mM of acetate and/or pharmaceutically acceptable acetate salt;
(c) about 6% (w/v) to about 10% (w/v) of sucrose;
(d) about 7 μg/mL to about 33 μg/mL of ethylenediaminetetraacetic acid (EDTA);
(e) about 0.01% to about 0.07% polysorbate 20; and
(f) a pH from about 4.7 to about 5.7.

In some embodiments, provided herein are aqueous pharmaceutical compositions comprising:
(a) a concentration of about 0.5 mg/mL to about 3.5 mg/mL of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
   (5) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
   (6) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (7) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
   (8) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
(f) about 10 mM to about 20 mM of acetate and/or pharmaceutically acceptable acetate salt;
(g) about 6% (w/v) to about 10% (w/v) of sucrose;
(h) about 7 μg/mL to about 33 μg/mL of ethylenediaminetetraacetic acid (EDTA);
(i) about 0.01% to about 0.07% polysorbate 20; and
(j) a pH from about 4.7 to about 5.7.

In some embodiments, provided herein are aqueous pharmaceutical compositions comprising:
(a) a concentration of about 25 mg/mL to about 55 mg/mL of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
(b) about 10 mM to about 20 mM of acetate and/or pharmaceutically acceptable acetate salt;
(c) about 6% (w/v) to about 10% (w/v) of sucrose;
(d) about 7 μg/mL to about 33 μg/mL of ethylenediaminetetraacetic acid (EDTA);
(e) about 0.01% to about 0.07% polysorbate 20; and
(f) a pH from about 4.7 to about 5.7.

In some embodiments, the GPRC5D/CD3 bispecific antibody comprises a GPRC5D binding domain comprising a VH having the HCDR1 of SEQ ID NO: 1, the HCDR2 of SEQ ID NO: 2, the HCDR3 of SEQ ID NO: 3, and a VL having the LCDR1 of SEQ ID NO: 4, the LCDR2 of SEQ ID NO: 5 and the LCDR3 of SEQ ID NO: 6, and a CD3 binding domain comprising a VH having the HCDR1 of SEQ ID NO: 11, the HCDR2 of SEQ ID NO: 12, the HCDR3 of SEQ ID NO: 13, and a VL having the LCDR1 of SEQ ID NO: 14, the LCDR2 of SEQ ID NO: 15 and the LCDR3 of SEQ ID NO: 16. The HCDRs and LCDRs of the GPRC5D/CD3 bispecific antibody are recited in Table 2 below:

TABLE 2
Exemplary CDRs of GPRC5D/CD3 bispecific antibody

| Binding Arm | Region | Sequence | SEQ ID NO: |
|---|---|---|---|
| GPRC5D | HCDR1 | GYTMN | 1 |
| | HCDR2 | LINPYNSDTNYAQKLQG | 2 |
| | HCDR3 | VALRVALDY | 3 |
| | LCDR1 | KASQNVATHVG | 4 |
| | LCDR2 | SASYRYS | 5 |
| | LCDR3 | QQYNRYPYT | 6 |
| CD3 | HCDR1 | TYAMN | 11 |
| | HCDR2 | RIRSKYNNYATYYAASVKG | 12 |
| | HCDR3 | HGNFGNSYVSWFAY | 13 |
| | LCDR1 | RSSTGAVTTSNYAN | 14 |
| | LCDR2 | GTNKRAP | 15 |
| | LCDR3 | ALWYSNLWV | 16 |

The CDRs recited in the table above are of the Kabat numbering system. However, as provided for herein, the CDRs of the present disclosure may be provided by any appropriate numbering system, such as any of the Kabat, Chothia, IMGT, or AbM numbering systems. Tables 3-5 below provide exemplary CDRs utilizing the Chothia, AbM, and IMGT numbering systems:

TABLE 3
Exemplary CDRs of GPRC5D/CD3 bispecific
antibody-Chothia numbering system:

| Binding Arm | Region | Sequence | SEQ ID NO: |
|---|---|---|---|
| GPRC5D | HCDR1 | GYSFTGY | 24 |
| | HCDR2 | NPYNSD | 25 |
| | HCDR3 | VALRVALDY | 3 |
| | LCDR1 | KASQNVATHVG | 4 |
| | LCDR2 | SASYRYS | 5 |
| | LCDR3 | QQYNRYPYT | 6 |
| CD3 | HCDR1 | GFTFNTY | 26 |
| | HCDR2 | RSKYNNYA | 27 |
| | HCDR3 | HGNFGNSYVSWFAY | 13 |
| | LCDR1 | RSSTGAVTTSNYAN | 14 |
| | LCDR2 | GTNKRAP | 15 |
| | LCDR3 | ALWYSNLWV | 16 |

TABLE 4
Exemplary CDRs of GPRC5D/CD3 bispecific
antibody-AbM numbering system:

| Binding Arm | Region | Sequence | SEQ ID NO: |
|---|---|---|---|
| GPRC5D | HCDR1 | GYSFTGYTMN | 28 |
| | HCDR2 | LINPYNSDTN | 29 |
| | HCDR3 | VALRVALDY | 3 |
| | LCDR1 | KASQNVATHVG | 4 |
| | LCDR2 | SASYRYS | 5 |
| | LCDR3 | QQYNRYPYT | 6 |
| CD3 | HCDR1 | GFTFNTYAMN | 30 |
| | HCDR2 | RIRSKYNNYATY | 31 |
| | HCDR3 | HGNFGNSYVSWFAY | 11 |
| | LCDR1 | RSSTGAVTTSNYAN | 12 |
| | LCDR2 | GTNKRAP | 13 |
| | LCDR3 | ALWYSNLWV | 14 |

TABLE 5
Exemplary CDRs of GPRC5D/CD3 bispecific
antibody-IMGT numbering system:

| Binding Arm | Region | Sequence | SEQ ID NO: |
|---|---|---|---|
| GPRC5D | HCDR1 | GYSFTGYT | 32 |
| | HCDR2 | INPYNSDT | 33 |
| | HCDR3 | ARVALRVALDY | 34 |
| | LCDR1 | QNVATH | 35 |
| | LCDR2 | SAS | NA |
| | LCDR3 | QQYNRYPYT | 6 |
| CD3 | HCDR1 | GFTFNTYA | 36 |
| | HCDR2 | IRSKYNNYAT | 37 |
| | HCDR3 | ARHGNFGNSYVSWFAY | 38 |
| | LCDR1 | TGAVTTSNY | 39 |
| | LCDR2 | GTN | NA |
| | LCDR3 | ALWYSNLWV | 16 |

In some embodiments, the GPRC5D/CD3 bispecific antibody comprises a GPRC5D binding domain comprising a VH having the HCDR1 of SEQ ID NO: 1, the HCDR2 of SEQ ID NO: 2, the HCDR3 of SEQ ID NO: 3, and a VL having the LCDR1 of SEQ ID NO: 4, the LCDR2 of SEQ ID NO: 5 and the LCDR3 of SEQ ID NO: 6, and a CD3 binding domain comprising a VH having the HCDR1 of SEQ ID NO: 11, the HCDR2 of SEQ ID NO: 12, the HCDR3 of SEQ ID NO: 13, and a VL having the LCDR1 of SEQ ID NO: 14, the LCDR2 of SEQ ID NO: 15 and the LCDR3 of SEQ ID NO: 16.

In some embodiments, the GPRC5D/CD3 bispecific antibody comprises a GPRC5D binding domain comprising a VH having the HCDR1 of SEQ ID NO: 24, the HCDR2 of SEQ ID NO: 25, the HCDR3 of SEQ ID NO: 3, and a VL having the LCDR1 of SEQ ID NO: 4, the LCDR2 of SEQ ID NO: 5 and the LCDR3 of SEQ ID NO: 6, and a CD3 binding domain comprising a VH having the HCDR1 of SEQ ID NO: 26, the HCDR2 of SEQ ID NO: 27, the HCDR3 of SEQ ID NO: 13, and a VL having the LCDR1 of SEQ ID NO: 14, the LCDR2 of SEQ ID NO: 15 and the LCDR3 of SEQ ID NO: 16.

In some embodiments, the GPRC5D/CD3 bispecific antibody comprises a GPRC5D binding domain comprising a VH having the HCDR1 of SEQ ID NO: 28, the HCDR2 of SEQ ID NO: 29, the HCDR3 of SEQ ID NO: 3, and a VL having the LCDR1 of SEQ ID NO: 4, the LCDR2 of SEQ ID NO: 5 and the LCDR3 of SEQ ID NO: 6, and a CD3 binding domain comprising a VH having the HCDR1 of SEQ ID NO: 30, the HCDR2 of SEQ ID NO: 31, the HCDR3 of SEQ ID NO: 13, and a VL having the LCDR1 of SEQ ID NO: 14, the LCDR2 of SEQ ID NO: 15 and the LCDR3 of SEQ ID NO: 16.

In some embodiments, the GPRC5D/CD3 bispecific antibody comprises a GPRC5D binding domain comprising a VH having the HCDR1 of SEQ ID NO: 32, the HCDR2 of SEQ ID NO: 33, the HCDR3 of SEQ ID NO: 34, and a VL having the LCDR1 of SEQ ID NO: 35, a LCDR2 having the amino acid sequence of SAS and the LCDR3 of SEQ ID NO: 6, and a CD3 binding domain comprising a VH having the HCDR1 of SEQ ID NO: 36, the HCDR2 of SEQ ID NO: 37, the HCDR3 of SEQ ID NO: 38, and a VL having the LCDR1 of SEQ ID NO: 39, a LCDR2 having the amino acid sequence of GTN and the LCDR3 of SEQ ID NO: 16.

In certain embodiments, the bispecific GPRC5D/CD3 antibody comprises a VH1 having the amino acid sequence of SEQ ID NO:7 and a VL1 having the amino acid sequence of SEQ ID NO:8. In certain embodiments, the bispecific GPRC5D/CD3 antibody comprises a HC1 having the amino acid sequence of SEQ ID NO:9, and a LC1 having the amino acid sequence of SEQ ID NO:10. In certain embodiments, the bispecific GPRC5D/CD3 antibody comprises a VH2 having the amino acid sequence of SEQ ID NO:17, and a VL2 having the amino acid sequence of SEQ ID NO:18. In certain embodiments, the bispecific GPRC5D/CD3 antibody comprises a HC2 having the amino acid sequence of SEQ ID NO:19, and a LC2 having the amino acid sequence of SEQ ID NO:20.

In some embodiments, the GPRC5D binding arm of the GPRC5D/CD3 bispecific antibody and the CD3 binding arm of the GPRC5D/CD3 bispecific antibody comprise the amino acid sequences as provided for in Tables 6a and 6b.

TABLE 6a
Sequences of the GPRC5D binding arm of a GPRC5D/CD3 bispecific antibody.

| | Region | Sequence | SEQ ID NO: |
|---|---|---|---|
| GC5B596 | HCDR1 | GYTMN | 1 |
| | HCDR2 | LINPYNSDTNYAQKLQG | 2 |
| | HCDR3 | VALRVALDY | 3 |
| | LCDR1 | KASQNVATHVG | 4 |
| | LCDR2 | SASYRYS | 5 |
| | LCDR3 | QQYNRYPYT | 6 |
| | VH | QVQLVQSGAEVKKPGASVKVSCKASGYSFTGYTMN | 7 |
| | | WVRQAPGQGLEWMGLINPYNSDTNYAQKLQGRVT | |
| | | MTTDTSTSTAYMELRSLRSDDTAVYYCARVALRVA | |
| | | LDYWGQGTLVTVSS | |
| | VL | DIQMTQSPSSLSASVGDRVTITCKASQNVATHVGWY | 8 |
| | | QQKPGKAPKRLIYSASYRYSGVPSRFSGSGSGTEFTL | |
| | | TISNLQPEDFATYYCQQYNRYPYTFGQGTKLEIK | |
| | HC | QVQLVQSGAEVKKPGASVKVSCKASGYSFTGYTMN | 9 |
| | | WVRQAPGQGLEWMGLINPYNSDTNYAQKLQGRVT | |
| | | MTTDTSTSTAYMELRSLRSDDTAVYYCARVALRVA | |
| | | LDYWGQGTLVTVSSASTKGPSVFPLAPCSRSTSESTA | |
| | | ALGCLVKDYFPEPVTVSWNSGALTSGVHTFPAVLQS | |
| | | SGLYSLSSVVTVPSSSLGTKTYTCNVDHKPSNTKVD | |
| | | KRVESKYGPPCPPCPAPEAAGGPSVFLFPPKPKDTLM | |
| | | ISRTPEVTCVVVDVSQEDPEVQFNWYVDGVEVHNA | |
| | | KTKPREEQFNSTYRVVSVLTVLHQDWLNGKEYKCK | |
| | | VSNKGLPSSIEKTISKAKGQPREPQVYTLPPSQEEMT | |
| | | KNQVSLTCLVKGFYPSDIAVEWESNGQPENNYKTTP | |
| | | PVLDSDGSFFLYSRLTVDKSRWQEGNVFSCSVMHE | |
| | | ALHNHYTQKSLSLSLGK | |
| | LC | DIQMTQSPSSLSASVGDRVTITCKASQNVATHVGWY | 10 |
| | | QQKPGKAPKRLIYSASYRYSGVPSRFSGSGSGTEFTL | |
| | | TISNLQPEDFATYYCQQYNRYPYTFGQGTKLEIKRT | |
| | | VAAPSVFIFPPSDEQLKSGTASVVCLLNNFYPREAKV | |
| | | QWKVDNALQSGNSQESVTEQDSKDSTYSLSSTLTLS | |
| | | KADYEKHKVYACEVTHQGLSSPVTKSFNRGEC | |

TABLE 6b
Sequences of the CD3 binding arm of a GPRC5D x CD3 bispecific antibody.

| | Region | Sequence | SEQ ID NO: |
|---|---|---|---|
| CD3B219 | HCDR1 | TYAMN | 11 |
| | HCDR2 | RIRSKYNNYATYYAASVKG | 12 |
| | HCDR3 | HGNFGNSYVSWFAY | 13 |
| | LCDR1 | RSSTGAVTTSNYAN | 14 |
| | LCDR2 | GTNKRAP | 15 |
| | LCDR3 | ALWYSNLWV | 16 |
| | VH | EVQLVESGGGLVQPGGSLRLSCAASGFTFNTYAMN | 17 |
| | | WVRQAPGKGLEWVARIRSKYNNYATYYAASVKG | |
| | | RFTISRDDSKNSLYLQMNSLKTEDTAVYYCARHGN | |
| | | FGNSYVSWFAYWGQGTLVTVSS | |
| | VL | QTVVTQEPSLTVSPGGTVTLTCRSSTGAVTTSNYAN | 18 |
| | | WVQQKPGQAPRGLIGGTNKRAPGTPARFSGSLLGG | |
| | | KAALTLSGVQPEDEAEYYCALWYSNLWVFGGGTK | |
| | | LTVLGQP | |
| | HC | EVQLVESGGGLVQPGGSLRLSCAASGFTFNTYAMN | 19 |
| | | WVRQAPGKGLEWVARIRSKYNNYATYYAASVKG | |
| | | RFTISRDDSKNSLYLQMNSLKTEDTAVYYCARHGN | |
| | | FGNSYVSWFAYWGQGTLVTVSSASTKGPSVFPLAP | |
| | | CSRSTSESTAALGCLVKDYFPEPVTVSWNSGALTSG | |
| | | VHTFPAVLQSSGLYSLSSVVTVPSSSLGTKTYTCNV | |
| | | DHKPSNTKVDKRVESKYGPPCPPCPAPEAAGGPSV | |
| | | FLFPPKPKDTLMISRTPEVTCVVVDVSQEDPEVQFN | |
| | | WYVDGVEVHNAKTKPREEQFNSTYRVVSVLTVLH | |
| | | QDWLNGKEYKCKVSNKGLPSSIEKTISKAKGQPRE | |
| | | PQVYTLPPSQEEMTKNQVSLTCLVKGFYPSDIAVE | |
| | | WESNGQPENNYKTTPPVLDSDGSFLLYSKLTVDKS | |
| | | RWQEGNVFSCSVMHEALHNHYTQKSLSLSLGK | |
| | LC | QTVVTQEPSLTVSPGGTVTLTCRSSTGAVTTSNYAN | 20 |
| | | WVQQKPGQAPRGLIGGTNKRAPGTPARFSGSLLGG | |
| | | KAALTLSGVQPEDEAEYYCALWYSNLWVFGGGTK | |
| | | LTVLGQPKAAPSVTLFPPSSEELQANKATLVCLISDF | |
| | | YPGAVTVAWKADSSPVKAGVETTTPSKQSNNKYA | |
| | | ASSYLSLTPEQWKSHRSYSCQVTHEGSTVEKTVAP | |
| | | TECS | |

In some embodiments, the bispecific GPRC5D/CD3 antibody can, for example, be talquetamab. In some embodiments, talquetamab comprises a first heavy chain (HC1), a first light chain (LC1), a second heavy chain (HC2), and a second light chain (LC2), wherein the HC1 is associated with LC1 and the HC2 is associated with LC2, wherein HC1 and LC1 form a first antigen-binding site that immunospecifically binds to GPRC5D and wherein HC2 and LC2 form a second antigen-binding site that immunospecifically binds to CD3. In some embodiments, talquetamab comprises a HC1 of SEQ ID NO: 9, a LC1 of SEQ ID NO: 10, a HC2 of SEQ TD NO: 19, and a LC2 of SEQ ID NO: 20. In some embodiments, the GPRC5D arm and the CD3 arm of talquetamab form a functional bispecific antibody through an interaction between their respective Fc domains.

In some embodiments, the bispecific GPRC5D/CD3 antibody has a concentration of about 0.5 mg/mL to about 100 mg/mL, about 30.0 mg/mL to about 90.0 mg/mL, about 40 mg/mL to about 90.0 mg/mL, about 50 mg/mL to about 90.0 mg/mL, or about 60.0 mg/mL to about 90.0 mg/mL, or any value or range in between. In some embodiments, the bispecific GPRC5D/CD3 antibody can, for example, have a concentration of about 10 mg/mL, about 20 mg/mL, about 30 mg/mL, about 40 mg/mL, about 50 mg/mL, about 60 mg/mL, about 70 mg/mL, about 80 mg/mL, or about 90 mg/mL, or any value in between. In some embodiments, the bispecific GPRC5D/CD3 antibody can, for example, have a concentration of about 2 mg/mL, about 30 mg/mL, about 40 mg/mL, about 60 mg/mL, or about 90 mg/mL.

In some embodiments, the bispecific GPRC5D/CD3 antibody has a concentration of about 0.5 mg/mL to about 3.5 mg/mL, about 1.0 mg/mL to about 3.0 mg/mL, or about 1.5 mg/mL to about 2.5 mg/mL, or any value or range in between.

In some embodiments, the bispecific GPRC5D/CD3 antibody has a concentration of about 0.5 mg/mL to about 3.5 mg/mL. In some embodiments, the bispecific GPRC5D/CD3 antibody has a concentration of about 1.0 mg/mL to about 3.5 mg/mL. In some embodiments, the bispecific GPRC5D/CD3 antibody has a concentration of about 1.5 mg/mL to about 3.5 mg/mL. In some embodiments, the bispecific GPRC5D/CD3 antibody has a concentration of about 2.0 mg/mL to about 3.5 mg/mL. In some embodiments, the bispecific GPRC5D/CD3 antibody has a concentration of about 2.5 mg/mL to about 3.5 mg/mL. In some embodiments, the bispecific GPRC5D/CD3 antibody has a concentration of about 3.0 mg/mL to about 3.5 mg/mL.

In some embodiments, the bispecific GPRC5D/CD3 antibody has a concentration of about 0.5 mg/mL to about 3.5 mg/mL. In some embodiments, the bispecific GPRC5D/CD3 antibody has a concentration of about 0.5 mg/mL to about 3.0 mg/mL. In some embodiments, the bispecific GPRC5D/CD3 antibody has a concentration of about 0.5 mg/mL to about 2.5 mg/mL. In some embodiments, the bispecific GPRC5D/CD3 antibody has a concentration of about 0.5 mg/mL to about 2.0 mg/mL. In some embodiments, the bispecific GPRC5D/CD3 antibody has a concentration of about 0.5 mg/mL to about 1.5 mg/mL. In some embodiments, the bispecific GPRC5D/CD3 antibody has a concentration of about 0.5 mg/mL to about 1.0 mg/mL.

In some embodiments, the bispecific GPRC5D/CD3 antibody can, for example, have a concentration of about 0.5 mg/mL, about 1 mg/mL, about 1.5 mg/mL, about 2 mg/mL, about 2.5 mg/mL, about 3 mg/mL, or about 3.5 mg/mL, or any value in between. In some embodiments, the bispecific GPRC5D/CD3 antibody has a concentration of about 0.5 mg/mL. In some embodiments, the bispecific GPRC5D/CD3 antibody has a concentration of about 1.0 mg/mL. In some embodiments, the bispecific GPRC5D/CD3 antibody has a concentration of about 1.5 mg/mL. In some embodiments, the bispecific GPRC5D/CD3 antibody has a concentration of about 2 mg/mL. In some embodiments, the bispecific GPRC5D/CD3 antibody has a concentration of about 2.5 mg/mL. In some embodiments, the bispecific GPRC5D/CD3 antibody has a concentration of about 3 mg/mL. In some embodiments, the bispecific GPRC5D/CD3 antibody has a concentration of about 3.5 mg/mL.

In some embodiments, the bispecific GPRC5D/CD3 antibody has a concentration of about 25 mg/mL to about 55 mg/mL, about 30 mg/mL to about 50 mg/mL, about 35 mg/mL to about 45 mg/mL, or about 38 mg/mL to about 42 mg/mL, or any value or range in between.

In some embodiments, the bispecific GPRC5D/CD3 antibody has a concentration of about 25 mg/mL to about 55 mg/mL. In some embodiments, the bispecific GPRC5D/CD3 antibody has a concentration of about 30 mg/mL to about 55 mg/mL. In some embodiments, the bispecific GPRC5D/CD3 antibody has a concentration of about 35 mg/mL to about 55 mg/mL. In some embodiments, the bispecific GPRC5D/CD3 antibody has a concentration of about 40 mg/mL to about 55 mg/mL. In some embodiments, the bispecific GPRC5D/CD3 antibody has a concentration of about 45 mg/mL to about 55 mg/mL. In some embodiments, the bispecific GPRC5D/CD3 antibody has a concentration of about 50 mg/mL to about 55 mg/mL.

In some embodiments, the bispecific GPRC5D/CD3 antibody has a concentration of about 25 mg/mL to about 55 mg/mL. In some embodiments, the bispecific GPRC5D/CD3 antibody has a concentration of about 25 mg/mL to about 50 mg/mL. In some embodiments, the bispecific GPRC5D/CD3 antibody has a concentration of about 25 mg/mL to about 45 mg/mL. In some embodiments, the bispecific GPRC5D/CD3 antibody has a concentration of about 25 mg/mL to about 40 mg/mL. In some embodiments, the bispecific GPRC5D/CD3 antibody has a concentration of about 25 mg/mL to about 35 mg/mL. In some embodiments, the bispecific GPRC5D/CD3 antibody has a concentration of about 25 mg/mL to about 30 mg/mL.

In some embodiments, the bispecific GPRC5D/CD3 antibody can, for example, have a concentration of about 25 mg/mL, about 30 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 50 mg/mL, or about 55 mg/mL, or any value in between. In some embodiments, the bispecific GPRC5D/CD3 antibody has a concentration of about 25 mg/mL. In some embodiments, the bispecific GPRC5D/CD3 antibody has a concentration of about 30 mg/mL. In some embodiments, the bispecific GPRC5D/CD3 antibody has a concentration of about 35 mg/mL. In some embodiments, the bispecific GPRC5D/CD3 antibody has a concentration of about 36 mg/mL. In some embodiments, the bispecific GPRC5D/CD3 antibody has a concentration of about 37 mg/mL. In some embodiments, the bispecific GPRC5D/CD3 antibody has a concentration of about 38 mg/mL. In some embodiments, the bispecific GPRC5D/CD3 antibody has a concentration of about 39 mg/mL. In some embodiments, the bispecific GPRC5D/CD3 antibody has a concentration of about 40 mg/mL. In some embodiments, the bispecific GPRC5D/CD3 antibody has a concentration of about 41 mg/mL. In some embodiments, the bispecific GPRC5D/CD3 antibody has a concentration of about 42 mg/mL. In some embodiments, the bispecific GPRC5D/CD3 antibody has a concentration of about 43 mg/mL. In some embodiments, the bispecific GPRC5D/CD3 antibody has a concentration of about 44 mg/mL. In some embodiments, the bispecific GPRC5D/CD3 antibody has a concentration of about 45 mg/mL. In some embodiments, the bispecific GPRC5D/CD3 antibody has a concentration of about 50 mg/mL. In some embodiments, the bispecific GPRC5D/CD3 antibody has a concentration of about 55 mg/mL.

In some embodiments, the GPRC5D/CD3 antibody can, for example, be talquetamab. In some embodiments, talquetamab is present at a concentration of about 0.5 mg/mL to about 3.5 mg/mL, about 1.0 mg/mL to about 3.0 mg/mL, or about 1.5 mg/mL to about 2.5 mg/mL, or any value or range in between.

In some embodiments, talquetamab is present at a concentration of about 0.5 mg/mL to about 3.5 mg/mL. In some embodiments, talquetamab is present at a concentration of about 1.0 mg/mL to about 3.5 mg/mL. In some embodiments, talquetamab is present at a concentration of about 1.5 mg/mL to about 3.5 mg/mL. In some embodiments, talquetamab is present at a concentration of about 2.0 mg/mL to about 3.5 mg/mL. In some embodiments, talquetamab is present at a concentration of about 2.5 mg/mL to about 3.5 mg/mL. In some embodiments, talquetamab is present at a concentration of about 3.0 mg/mL to about 3.5 mg/mL.

In some embodiments, talquetamab is present at a concentration of about 0.5 mg/mL to about 3.5 mg/mL. In some embodiments, talquetamab is present at a concentration of about 0.5 mg/mL to about 3.0 mg/mL. In some embodiments, talquetamab is present at a concentration of about 0.5 mg/mL to about 2.5 mg/mL. In some embodiments, talquetamab is present at a concentration of about 0.5 mg/mL to about 2.0 mg/mL. In some embodiments, talquetamab is present at a concentration of about 0.5 mg/mL to about 1.5 mg/mL. In some embodiments, talquetamab is present at a concentration of about 0.5 mg/mL to about 1.0 mg/mL.

In some embodiments, talquetamab is present at, for example, a concentration of about 0.5 mg/mL, about 1 mg/mL, about 1.5 mg/mL, about 2 mg/mL, about 2.5 mg/mL, about 3 mg/mL, or about 3.5 mg/mL, or any value in between. In some embodiments, talquetamab is present at a concentration of about 0.5 mg/mL. In some embodiments, talquetamab is present at a concentration of about 1.0 mg/mL. In some embodiments, talquetamab is present at a concentration of about 1.5 mg/mL. In some embodiments talquetamab is present at a concentration of about 2 mg/mL. In some embodiments, talquetamab is present at a concentration of about 2.5 mg/mL. In some embodiments talquetamab is present at a concentration of about 3 mg/mL. In some embodiments, talquetamab is present at a concentration of about 3.5 mg/mL.

In some embodiments, talquetamab is present at a concentration of about 25 mg/mL to about 55 mg/mL, about 30 mg/mL to about 50 mg/mL, about 35 mg/mL to about 45 mg/mL, or about 38 mg/mL to about 42 mg/mL, or any value or range in between.

In some embodiments, talquetamab is present at a concentration of about 25 mg/mL to about 55 mg/mL. In some embodiments, talquetamab is present at a concentration of about 30 mg/mL to about 55 mg/mL. In some embodiments, talquetamab is present at a concentration of about 35 mg/mL to about 55 mg/mL. In some embodiments talquetamab is present at a concentration of about 40 mg/mL to about 55 mg/mL. In some embodiments, talquetamab is present at a concentration of about 45 mg/mL to about 55 mg/mL. In some embodiments, talquetamab is present at a concentration of about 50 mg/mL to about 55 mg/mL.

In some embodiments talquetamab is present at a concentration of about 25 mg/mL to about 55 mg/mL. In some embodiments, talquetamab is present at a concentration of about 25 mg/mL to about 50 mg/mL. In some embodiments talquetamab is present at a concentration of about 25 mg/mL to about 45 mg/mL. In some embodiments, talquetamab is present at a concentration of about 25 mg/mL to about 40 mg/mL. In some embodiments, talquetamab is present at a concentration of about 25 mg/mL to about 35 mg/mL. In some embodiments, talquetamab is present at a concentration of about 25 mg/mL to about 30 mg/mL.

In some embodiments, talquetamab is present at, for example, a concentration of about 25 mg/mL, about 30 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 50 mg/mL, or about 55 mg/mL, or any value in between. In some embodiments talquetamab is present at a concentration of about 25 mg/mL. In some embodiments, talquetamab is present at a concentration of about 30 mg/mL. In some embodiments, talquetamab is present at a concentration of about 35 mg/mL. In some embodiments, talquetamab is present at a concentration of about 36 mg/mL. In some embodiments, talquetamab is present at a concentration of about 37 mg/mL. In some embodiments, talquetamab is present at a concentration of about 38 mg/mL. In some embodiments, talquetamab is present at a concentration of about 39 mg/mL. In some embodiments, talquetamab is present at a concentration of about 40 mg/mL. In some embodiments talquetamab is present at a concentration of about 41 mg/mL. In some embodiments talquetamab is present at a concentration of about 42 mg/mL. In some embodiments, talquetamab is present at a concentration of about 43 mg/mL. In some embodiments, talquetamab is present at a concentration of about 44 mg/mL. In some embodiments, talquetamab is present at a concentration of about 45 mg/mL. In some embodiments, talquetamab is present at a concentration of about 50 mg/mL. In some embodiments talquetamab is present at a concentration of about 55 mg/mL.

In some embodiments, the composition comprises about 10 mM to about 20 mM, about 12 mM to about 18 mM, or about 14 mM to about 16 mM, or any value or range in between of acetate and/or a pharmaceutically acceptable acetate salt.

In some embodiments, the composition comprises about 10 mM to about 20 mM of acetate and/or a pharmaceutically acceptable acetate salt. In some embodiments, the composition comprises about 11 mM to about 20 mM of acetate and/or a pharmaceutically acceptable acetate salt. In some embodiments, the composition comprises about 12 mM to about 20 mM of acetate and/or a pharmaceutically acceptable acetate salt. In some embodiments, the composition comprises about 13 mM to about 20 mM of acetate and/or a pharmaceutically acceptable acetate salt. In some embodiments, the composition comprises about 14 mM to about 20 mM of acetate and/or a pharmaceutically acceptable acetate salt. In some embodiments, the composition comprises about 15 mM to about 20 mM of acetate and/or a pharmaceutically acceptable acetate salt. In some embodiments, the composition comprises about 16 mM to about 20 mM of acetate and/or a pharmaceutically acceptable acetate salt. In some embodiments, the composition comprises about 17 mM to about 20 mM of acetate and/or a pharmaceutically acceptable acetate salt. In some embodiments, the composition comprises about 18 mM to about 20 mM of acetate and/or a pharmaceutically acceptable acetate salt. In some embodiments, the composition comprises about 19 mM to about 20 mM of acetate and/or a pharmaceutically acceptable acetate salt.

In some embodiments, the composition comprises about 10 mM to about 20 mM of acetate and/or a pharmaceutically acceptable acetate salt. In some embodiments, the composition comprises about 10 mM to about 19 mM of acetate and/or a pharmaceutically acceptable acetate salt. In some embodiments, the composition comprises about 10 mM to about 18 mM of acetate and/or a pharmaceutically acceptable acetate salt. In some embodiments, the composition comprises about 10 mM to about 17 mM of acetate and/or a pharmaceutically acceptable acetate salt. In some embodiments, the composition comprises about 10 mM to about 16 mM of acetate and/or a pharmaceutically acceptable acetate salt. In some embodiments, the composition comprises about 10 mM to about 15 mM of acetate and/or a pharmaceutically acceptable acetate salt. In some embodiments, the composition comprises about 10 mM to about 14 mM of acetate and/or a pharmaceutically acceptable acetate salt. In some embodiments, the composition comprises about 10 mM to about 13 mM of acetate and/or a pharmaceutically acceptable acetate salt. In some embodiments, the composition comprises about 10 mM to about 12 mM of acetate and/or a pharmaceutically acceptable acetate salt. In some embodiments, the composition comprises about 10 mM to about 11 mM of acetate and/or a pharmaceutically acceptable acetate salt.

In some embodiments, the composition can, for example, comprise about 10 mM, about 11 mM, about 12 mM, about 13 mM, about 14 mM, about 15 mM, about 16 mM, about 17 mM, about 18 mM, about 19 mM, or about 20 mM, or any value in between of acetate and/or a pharmaceutically acceptable acetate salt. In some embodiments, the composition comprises about 10 mM acetate or a pharmaceutically acceptable acetate salt. In some embodiments, the composition comprises about 11 mM acetate or a pharmaceutically acceptable acetate salt. In some embodiments, the composition comprises about 12 mM acetate or a pharmaceutically acceptable acetate salt. In some embodiments, the composition comprises about 13 mM acetate or a pharmaceutically acceptable acetate salt. In some embodiments, the composition comprises about 14 mM acetate or a pharmaceutically acceptable acetate salt. In some embodiments, the composition comprises about 15 mM acetate or a pharmaceutically acceptable acetate salt. In some embodiments, the composition comprises about 16 mM acetate or a pharmaceutically acceptable acetate salt. In some embodiments, the composition comprises about 17 mM acetate or a pharmaceutically acceptable acetate salt. In some embodiments, the composition comprises about 18 mM acetate or a pharmaceutically acceptable acetate salt. In some embodiments, the composition comprises about 19 mM acetate or a pharmaceutically acceptable acetate salt. In some embodiments, the composition comprises about 20 mM acetate or a pharmaceutically acceptable acetate salt.

In some embodiments, the composition comprises about 6% (w/v) to about 10% (w/v) or about 7% (w/v) to about 9% (w/v), or any value or range in between, of sucrose.

In some embodiments, the composition comprises about 6% (w/v) to about 10% (w/v) sucrose. In some embodiments, the composition comprises about 7% (w/v) to about 10% (w/v) sucrose. In some embodiments, the composition comprises about 8% (w/v) to about 10% (w/v) sucrose. In some embodiments, the composition comprises about 9% (w/v) to about 10% (w/v) sucrose.

In some embodiments, the composition comprises about 6% (w/v) to about 10% (w/v) sucrose. In some embodiments, the composition comprises about 6% (w/v) to about 9% (w/v) sucrose. In some embodiments, the composition comprises about 6% (w/v) to about 8% (w/v) sucrose. In some embodiments, the composition comprises about 6% (w/v) to about 7% (w/v) sucrose.

In some embodiments, the composition can, for example comprise about 6% (w/v), about 7% (w/v), about 8% (w/v), about 9% (w/v), or about 10% (w/v), or any value in between of sucrose. In some embodiments, the composition comprises about 6% (w/v) sucrose. In some embodiments, the composition comprises about 7% (w/v) sucrose. In some embodiments, the composition comprises about 8% (w/v) sucrose. In some embodiments, the composition comprises about 9% (w/v) sucrose. In some embodiments, the composition comprises about 10% (w/v) sucrose.

In some embodiments, the composition comprises about 7 μg/mL to about 33 μg/mL, 12 μg/mL to about 28 μg/mL, or about 15 μg/mL to about 21 μg/mL, or any value or range in between, of EDTA.

In some embodiments, the composition comprises about 7 μg/mL to about 33 μg/mL EDTA. In some embodiments, the composition comprises about 12 μg/mL to about 33 μg/mL EDTA. In some embodiments, the composition comprises about 15 μg/mL to about 33 μg/mL EDTA. In some embodiments, the composition comprises about 16 μg/mL to about 33 μg/mL EDTA. In some embodiments, the composition comprises about 17 μg/mL to about 33 μg/mL EDTA. In some embodiments, the composition comprises about 18 μg/mL to about 33 μg/mL EDTA. In some embodiments, the composition comprises about 19 μg/mL to about 33 μg/mL EDTA. In some embodiments, the composition comprises about 20 μg/mL to about 33 μg/mL EDTA. In some embodiments, the composition comprises about 21 μg/mL to about 33 μg/mL EDTA. In some embodiments, the composition comprises about 24 μg/mL to about 33 μg/mL EDTA. In some embodiments, the composition comprises about 28 μg/mL to about 33 μg/mL EDTA.

In some embodiments, the composition comprises about 7 μg/mL to about 33 μg/mL EDTA. In some embodiments, the composition comprises about 7 μg/mL to about 28 μg/mL EDTA. In some embodiments, the composition comprises about 7 μg/mL to about 24 μg/mL EDTA. In some embodiments, the composition comprises about 7 μg/mL to about 21 μg/mL EDTA. In some embodiments, the composition comprises about 7 μg/mL to about 20 μg/mL EDTA. In some embodiments, the composition comprises about 7 μg/mL to about 19 μg/mL EDTA. In some embodiments, the composition comprises about 7 μg/mL to about 18 μg/mL EDTA. In some embodiments, the composition comprises about 7 μg/mL to about 17 μg/mL EDTA. In some embodiments, the composition comprises about 7 μg/mL to about 16 μg/mL EDTA. In some embodiments, the composition comprises about 7 μg/mL to about 15 μg/mL EDTA. In some embodiments, the composition comprises about 7 μg/mL to about 12 μg/mL EDTA.

In some embodiments, the composition can, for example, comprise about 7 μg/mL, 12 μg/mL, 15 μg/mL, 16 μg/mL, about 17 μg/mL, about 18 μg/mL, about 19 μg/mL, about 20 μg/mL, about 21 μg/mL, about 22 μg/mL, about 23 μg/mL, about 24 μg/mL, about 28 μg/mL, or about 33 μg/mL, or any value in between, of EDTA. In some embodiments, the composition comprises about 7 μg/mL EDTA. In some embodiments, the composition comprises about 12 μg/mL EDTA. In some embodiments, the composition comprises about 15 μg/mL EDTA. In some embodiments, the composition comprises about 16 μg/mL EDTA. In some embodiments, the composition comprises about 17 μg/mL EDTA. In some embodiments, the composition comprises about 18 μg/mL EDTA. In some embodiments, the composition comprises about 19 μg/mL EDTA. In some embodiments, the composition comprises about 20 μg/mL of EDTA. In some embodiments, the composition comprises about 21 μg/mL EDTA. In some embodiments, the composition comprises about 22 μg/mL EDTA. In some embodiments, the composition comprises about 23 μg/mL EDTA. In some embodiments, the composition comprises about 24 μg/mL EDTA. In some embodiments, the composition comprises about 28 μg/mL EDTA. In some embodiments, the composition comprises about 33 μg/mL EDTA.

In some embodiments, the composition comprises about 0.01% to about 0.07%, about 0.02% to about 0.06%, or about 0.03% to about 0.05%, or any value or range in between, of polysorbate 20 (PS20).

In some embodiments, the composition comprises about 0.01% to about 0.07% PS20. In some embodiments, the composition comprises about 0.02% to about 0.07% PS20. In some embodiments, the composition comprises about 0.03% to about 0.07% PS20. In some embodiments, the composition comprises about 0.04% to about 0.07% PS20. In some embodiments, the composition comprises about 0.05% to about 0.07% PS20. In some embodiments, the composition comprises about 0.06% to about 0.07% PS20.

In some embodiments, the composition comprises about 0.01% to about 0.07% PS20. In some embodiments, the composition comprises about 0.01% to about 0.06% PS20. In some embodiments, the composition comprises about 0.01% to about 0.05% PS20. In some embodiments, the composition comprises about 0.01% to about 0.04% PS20. In some embodiments, the composition comprises about 0.01% to about 0.03% PS20. In some embodiments, the composition comprises about 0.01% to about 0.02% PS20.

In some embodiments, the composition can, for example, comprise about 0.01%, about 0.02%, about 0.03%, about 0.04%, about 0.05%, about 0.06%, or about 0.07%, or any value in between of PS20. In some embodiments, the composition comprises about 0.01% PS20. In some embodiments, the composition comprises about 0.02% PS20. In some embodiments, the composition comprises about 0.03% PS20. In some embodiments, the composition comprises about 0.04% PS20. In some embodiments, the composition comprises about 0.05% PS20. In some embodiments, the composition comprises about 0.06% PS20. In some embodiments, the composition comprises about 0.07% PS20.

In some embodiments, the pH of the composition is about 4.7 to about 5.7, about 4.8 to about 5.6, about 4.9 to about 5.5, or any value or range in-between.

In some embodiments, the pH of the composition is about 4.7 to about 5.7. In some embodiments, the pH of the composition is about 4.8 to about 5.7. In some embodiments, the pH of the composition is about 4.9 to about 5.7. In some embodiments, the pH of the composition is about 5.0 to about 5.7. In some embodiments, the pH of the composition is about 5.1 to about 5.7. In some embodiments, the pH of the composition is about 5.2 to about 5.7. In some embodiments, the pH of the composition is about 5.3 to about 5.7. In some embodiments, the pH of the composition is about 5.4 to about 5.7. In some embodiments, the pH of the composition is about 5.5 to about 5.7. In some embodiments, the pH of the composition is about 5.6 to about 5.7.

In some embodiments, the pH of the composition is about 4.7 to about 5.7. In some embodiments, the pH of the composition is about 4.7 to about 5.6. In some embodiments, the pH of the composition is about 4.7 to about 5.5. In some embodiments, the pH of the composition is about 4.7 to about 5.4. In some embodiments, the pH of the composition is about 4.7 to about 5.3. In some embodiments, the pH of the composition is about 4.7 to about 5.2. In some embodiments, the pH of the composition is about 4.7 to about 5.1. In some embodiments, the pH of the composition is about 4.7 to about 5.0. In some embodiments, the pH of the composition is about 4.7 to about 4.9. In some embodiments, the pH of the composition is about 4.7 to about 4.8.

In some embodiments, the pH of the composition can, for example, be about 4.7 about 4.8, about 4.9, about 5.0, about 5.1, about 5.2, about 5.3, about 5.4, about 5.5, about 5.6, or about 5.7, or any value in between. In some embodiments, the pH of the composition is about 4.7. In some embodiments, the pH of the composition is about 4.8. In some embodiments, the pH of the composition is about 4.9. In some embodiments, the pH of the composition is about 5.0. In some embodiments, the pH of the composition is about 5.1. In some embodiments, the pH of the composition is about 5.2. In some embodiments, the pH of the composition is about 5.3. In some embodiments, the pH of the composition is about 5.4. In some embodiments, the pH of the composition is about 5.5. In some embodiments, the pH of the composition is about 5.6. In some embodiments, the pH of the composition is about 5.7

The stability of the presently disclosed aqueous pharmaceutical compositions, also referred to as drug product (DP), is determined based on specific amount or proportion of the GPRC5D/CD3 antibody and other constituents of the DP as provided herein (such as, but not limited to, acetate and/or pharmaceutically acceptable acetate salts, sucrose, PS20, and EDTA), as well as the assessment of various factors. These factors include but are not limited to the color of the solution, the pH, the turbidity, percentage of purity, number of subvisible particles, percentage of new peak(s), percentage of main component, percentage of high molecular weight species (HMWS), percentage of low molecular weight species (LMWS), percentage of sum of acidic peaks, percentage of sum of basic peaks, protein concentration, percentage of T-cell activation, and/or percentage of PS20.

Stable DP as disclosed herein should not be construed to require all the factors listed herein but rather at least one, at least two, or at least three or more of those factors. In some embodiments, the stable disclosed DP exhibits the following results for at least one, at least two, at least three or more of the factors listed in detail below herein. In some embodiments, the stable DP exhibits the following results for most of the factors listed in detail below herein. In some embodiments, the stable DP exhibits the following results for all the factors listed in detail below herein.

### Color of Solution

The Color of a DP solution is monitored and can be assessed to verify that the appearance of the solution is consistent with previous batches at release and over the shelf life. The color of the DP solution can reflect stability. In some embodiments, a stable DP is defined as having a color of solution spanning from colorless to about BY2 or less, to about BY4 or less, to about B2 or less, to about B4 or less, to about Y2 or less or to about Y4 or less as described in the European Pharmacopoeia 2.2.2, Degree of Coloration of Liquids European Pharmacopoeia (Ph. Eur.) 10th Edition monograph number 20202, July 2019.

In some embodiments, a stable DP is defined as having a color of solution of colorless to about BY2 or less, about B2 or less, about Y2 or less after storage for about 12 months or more and at a temperature of about 5° C., after storage for about 12 months or more and at a temperature of about 25° C., and/or after storage for about 2 years or more and at a temperature of about 5° C. In some embodiments, a stable DP is defined as having a color of solution of colorless to about BY4 or less, to about B4 or less, to about Y4 or less after storage for about 12 months or more and at a temperature of about 5° C., after storage for about 12 months or more and at a temperature of about 25° C., and/or after storage for about 2 years or more and at a temperature of about 5° C. In some embodiments, a stable DP is defined as having a color of solution of colorless to about BY5 or less, to about B5 or less, to about Y5 or less after storage for about 12 months or more and at a temperature of about 5° C., after storage for about 12 months or more and at a temperature of about 25° C., and/or after storage for about 2 years or more and at a temperature of about 5° C.

pH

Measuring the pH of the DP solution allows confirmation that it is consistent with previous DP batches at release and over the shelf life. The pH of the solution can reflect stability. In some embodiments, a stable DP is defined as having a pH of about: 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7.0. In some embodiments, the pH of the DP is about 5.2 after storage for about 12 months or more and at a temperature of about 5° C., after storage for about 12 months or more and at a temperature of about 25° C., and/or after storage for about 2 years or more and at a temperature of about 5° C. In some embodiments, the pH ranges from about 4.7 to about 5.7 after storage for about 12 months or more and at a temperature of about 5° C., after storage for about 12 months or more and at a temperature of about 25° C., and/or after storage for about 2 years or more and at a temperature of about 5° C. In some embodiments, a stable DP is defined as having a pH range from about 4.8 to about 5.6 after storage for about 12 months or more and at a temperature of about 5° C., after storage for about 12 months or more and at a temperature of about 25° C., and/or after storage for about 2 years or more and at a temperature of about 5° C. In some embodiments, a stable DP is defined as having a pH range from about 4.9 to about 5.5 after storage for about 12 months or more and at a temperature of about 5° C., after storage for about 12 months or more and at a temperature of about 25° C., and/or after storage for about 2 years or more and at a temperature of about 5° C.

### Turbidity

Turbidity allows measuring the presence of particles in the DP solution in order to ensure consistency with previous DP batches and applicable compendia guidance at release and over the shelf life. The turbidity of a solution can reflect stability. In some embodiments, a stable DP is defined as having a turbidity value of about: 0, 1, 2, 3, 4, 5, 6, 7, 8, 9, 10, 11, 12, 13, 14, 15, 16, 17, 18, 19, or 20 nephelometric turbidity units (NTU) after storage for about 12 months or more and at a temperature of about 5° C., after storage for about 12 months or more and at a temperature of about 25° C., and/or after storage for about 2 years or more and at a temperature of about 5° C. In some embodiments, a stable DP is defined as having a turbidity value of about or less than 18 NTU after storage for about 12 months or more and at a temperature of about 5° C., after storage for about 12 months or more and at a temperature of about 25° C., and/or after storage for about 2 years or more and at a temperature of about 5° C. In some embodiments, a stable DP is defined as having a turbidity value of about or less than 13 NTU after storage for about 12 months or more and at a temperature of about 5° C., after storage for about 12 months or more and at a temperature of about 25° C., and/or after storage for about 2 years or more and at a temperature of about 5° C. In some embodiments, a stable DP is defined as having a turbidity value of about or less than 8 NTU after storage for about 12 months or more and at a temperature of about 5° C., after storage for about 12 months or more and at a temperature of about 25° C., and/or after storage for about 2 years or more and at a temperature of about 5° C.

### Particle Analysis

The stability of the DP is set to a specific threshold of particles contamination based on the average number of sub-visible particles. In some embodiments, the average number of particles present in the DP units tested should not exceed 100, 200, 300, 400, 500, 600, 700, 800, 900, 1000, 2000, 3000, 4000, 5000, or 6000, per container for particle size equal to 10 μm or greater. In some embodiments, the average number of particles present in the DP units tested should not exceed 6000 per container for particle size equal to 10 μm or greater. In some embodiments, the average number of particles present in the DP units tested should not exceed 100, 200, 300, 400, 500, or 600, per container for particle size equal to 25 μm or greater. In some embodiments, the average number of particles present in the DP units tested should not exceed 600 per container for particle size equal to 25 μm or greater.

cSDS Conditions

Capillary SDS-PAGE (cSDS), like gel-based SDS-PAGE, is a method for separating denatured protein based on molecular weight. This process allows quantifying DP purity and monitoring its stability at release and over the shelf life.

In some embodiments, the DP stability is defined based upon various results of cSDS variables (e.g. percent purity or presence of new peak) where the cSDS was performed under reduced or non-reduced conditions after storage for about 12 months or more and at a temperature of about 5° C., after storage for about 12 months or more and at a temperature of about 25° C., and/or after storage for about 2 years or more and at a temperature of about 5° C.

In some embodiments, a stable DP is defined as having a percent purity under reduced conditions of about: 80%, 81%, 82%, 83%, 84%, 85%, 86%, 87%, 88%, 89%, 90%, 91%, 92%, 93%, 94%, 95%, 96%, 97%, 98%, 99% or about or equal to 100% or any value or range in between after storage of the DP for about 12 months or more and at a temperature of about 5° C., after storage of the DP for about 12 months or more and at a temperature of about 25° C., and/or after storage of the DP for about 2 years or more and at a temperature of about 5° C.

In some embodiments, a stable DP is defined as showing no new peak in the reduced cSDS results of more than 0.5%, 0.8%, 0.9%, 1.0%, 1.2%, 1.3%, 1.4%, 1.5%, 1.6%, 1.7%, 1.8%, 1.9% or more than 2% when compared to an untreated reference material after storage of the DP for about 12 months or more and at a temperature of about 5° C., after storage of the DP for about 12 months or more and at a temperature of about 25° C., and/or after storage of the DP for about 2 years or more and at a temperature of about 5° C.

In some embodiments, a stable DP is defined as having a percent purity under reduced conditions of about 90% or more and no new peak of more than 1.5% as compared to a reference material after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a percent purity of about 95% or more, and with no new peak of more than 1.2% compared to a reference material after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a percent purity of about 97% or more and with no new peak of more than 1.0% as compared to a reference material after storage of the DP for about 12 months or more and at a temperature of about 5° C., after storage of the DP for about 12 months or more and at a temperature of about 25° C., and/or after storage of the DP for about 2 years or more and at a temperature of about 5° C.

In some embodiments, a stable DP is defined as having a percent purity under non-reduced conditions about: 80%, 81%, 82%, 83%, 84%, 85%, 86%, 87%, 88%, 89%, 90%, 91%, 92%, 93%, 94%, 95%, 96%, 97%, 98%, 99% or about or equal to 100% or any range there in between after storage of the DP for about 12 months or more and at a temperature of about 5° C., after storage of the DP for about 12 months or more and at a temperature of about 25° C., and/or after storage of the DP for about 2 years or more and at a temperature of about 5° C.

In some embodiments, a stable DP is defined as showing no new peak in the non-reduced cSDS results of more than 0.5%, 0.8%, 0.9%, 1.0%, 1.2%, 1.3%, 1.4%, 1.5%, 1.6%, 1.7%, 1.8%, 1.9% or more than 2% when compared to an untreated reference material after storage of the DP for about 12 months or more and at a temperature of about 5° C., after storage of the DP for about 12 months or more and at a temperature of about 25° C., and/or after storage of the DP for about 2 years or more and at a temperature of about 5° C.

In some embodiments, a stable DP is defined as having a percent purity under non-reduced conditions of about 90% or more and no new peak of more than 1.5% as compared to a reference material after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a percent purity of about 94% or more, and with no new peak of more than 1.2% compared to a reference material after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a percent purity of about 96% or more and with no new peak of more than 1.0% as compared to a reference material after storage of the DP for about 12 months or more and at a temperature of about 5° C., after storage of the DP for about 12 months or more and at a temperature of about 25° C., and/or after storage of the DP for about 2 years or more and at a temperature of about 5° C.

Size-Exclusion HPLC (SE-HPLC) Results Consistent with Stability

SE-HPLC procedure allows assessing purity of the DP and monitoring its stability under non-denaturing conditions at release and over the shelf life.

In some embodiments, the DP stability is defined based upon various results of SE-HPLC variables such as the Main Component (MC), High Molecular Weight Species (HMWS), or Low Molecular Weight Species (LMWS)), after storage of the DP for about 12 months or more and at a temperature of about 5° C., after storage for about 12 months or more and at a temperature of about 25° C., and/or after storage for about 2 years or more and at a temperature of about 5° C.

In some embodiments, a stable DP is defined as having a MC of about: 80%, 81%, 82%, 83%, 84%, 85%, 86%, 87%, 88%, 89%, 90%, 91%, 92%, 93%, 94%, 95%, 96%, 97%, 98%, 99% or equal to about 100% or any value or range in between after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a MC of about 90% or more after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a MC of about 95% or more after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a MC of about 97% or more after storage of the DP for about 12 months or more and at a temperature of about 5° C., after storage of the DP for about 12 months or more and at a temperature of about 25° C., and/or after storage of the DP for about 2 years or more and at a temperature of about 5° C.

In some embodiments, a stable DP is defined as having a HMWS of about: 0.1%, 0.5%, 1%, 2%, 3%, 4%, 5%, 6%, 7%, 8%, 9%, 10%, 11%, 12%, 13%, 14%, 15% or any value or range in between after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a HMWS of about 10% or less after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a HMWS of about 5% or less after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a HMWS of about to 3% or less after storage of the DP for about 12 months or more and at a temperature of about 5° C., after storage of the DP for about 12 months or more and at a temperature of about 25° C., and/or after storage of the DP for about 2 years or more and at a temperature of about 5° C.

In some embodiments, a stable DP is defined as having a LMWS of about: 0.1%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%, or any value or range in between after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a LMWS of about 5% or less after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a LMWS of about 2% or less after storage of the DP for about 12 months or more and at a temperature of about 5° C., after storage of the DP for about 12 months or more and at a temperature of about 25° C., and/or after storage of the DP for about 2 years or more and at a temperature of about 5° C. In some embodiments, a stable DP is defined as having a LMWS of about 1% or less after storage of the DP for about 12 months or more and at a temperature of about 5° C., after storage of the DP for about 12 months or more and at a temperature of about 25° C., and/or after storage of the DP for about 2 years or more and at a temperature of about 5° C.

### Ion Exchange Chromatography (IEX)

The IEX method separates proteins on the basis of overall charge. This procedure allows monitoring the distribution of charge-based isoforms of the drug product at release and over the shelf life. In some embodiments, the DP stability is defined based upon various results of IEX variables such as the Main Peak (MP), the sum of acidic peaks or the sum of basic peaks, after DP storage for about 12 months or more and at a temperature of about 25° C., and/or after storage for about 2 years or more and at a temperature of about 5° C.

In some embodiments, a stable DP is defined as having a IEX with a MP of about: 50%, 55%, 60%, 65%, 70%, 75%, 80%, 85%, 90%, 95%, or 100% or any value or range there in between after DP storage for about 12 months or more and at a temperature of about 25° C., and/or after storage for about 2 years or more and at a temperature of about 5° C. In some embodiments, a stable DP is defined as having a IEX with a MP≥50% after DP storage for about 12 months or more and at a temperature of about 25° C., and/or after storage for about 2 years or more and at a temperature of about 5° C. In some embodiments, a stable DP is defined as having a IEX with a MP≥55% after DP storage for about 12 months or more and at a temperature of about 25° C., and/or after storage for about 2 years or more and at a temperature of about 5° C. In some embodiments, a stable DP is defined as having a IEX with a MP≥60% after DP storage for about 12 months or more and at a temperature of about 25° C., and/or after storage for about 2 years or more and at a temperature of about 5° C.

In some embodiments, a stable DP is defined as having a IEX with a sum of acidic peaks totaling to about: 1%, 5%, 10%, 15%, 20%, 25%, 30%, 35%, 40%, 45%, 50%, or any value or range there in between after DP storage for about 12 months or more and at a temperature of about 25° C., and/or after storage for about 2 years or more and at a temperature of about 5° C. In some embodiments, a stable DP is defined as having a IEX with a sum of acidic peaks ≤50% after DP storage for about 12 months or more and at a temperature of about 25° C., and/or after storage for about 2 years or more and at a temperature of about 5° C. In some embodiments, a stable DP is defined as having a IEX with a sum of acidic peaks totaling to about ≤40% after DP storage for about 12 months or more and at a temperature of about 25° C., and/or after storage for about 2 years or more and at a temperature of about 5° C. In some embodiments, a stable DP is defined as having a IEX with a sum of acidic peaks totaling to about ≤30% after DP storage for about 12 months or more and at a temperature of about 25° C., and/or after storage for about 2 years or more and at a temperature of about 5° C.

In some embodiments, a stable DP is defined as having a IEX with a sum of basic peaks totaling about: 1%, 2%, 3%, 4%, 5%, 6%, 7% 8%, 9%10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%, or any value or range there in between after DP storage for about 12 months or more and at a temperature of about 25° C., and/or after storage for about 2 years or more and at a temperature of about 5° C. In some embodiments, a stable DP is defined as having a IEX with a sum of basic peaks totaling about 25% or less after DP storage for about 12 months or more and at a temperature of about 25° C., and/or after storage for about 2 years or more and at a temperature of about 5° C. In some embodiments, a stable DP is defined as having a IEX with a sum of basic peaks totaling less than or about 15% after DP storage for about 12 months or more and at a temperature of about 25° C., and/or after storage for about 2 years or more and at a temperature of about 5° C. In some embodiments, a stable DP is defined as having a IEX with a sum of basic peaks totaling less than or about 10% after DP storage for about 12 months or more and at a temperature of about 25° C., and/or after storage for about 2 years or more and at a temperature of about 5° C.

### Protein Concentration

Protein concentration of the DP allows verifying that it is consistent with previous DP batches at release and over the shelf life. Quantification of protein concentration can be accomplished by measuring the UV light absorbance of the drug product solution at 280 nm (A280).

2 mg/mL DP Formulation

In some embodiments, a stable DP is defined as having a protein concentration of about: 1 mg/mL, 1.2 mg/mL, 1.4 mg/mL, 1.6 mg/mL, 1.8 mg/mL, 2 mg/mL, 2.2 mg/mL, 2.4 mg/mL, 2.6 mg/mL, 2.8 mg/mL, 3 mg/mL, or any value or range in between, after DP storage for about 12 months or more and at a temperature of about 25° C., and/or after storage for about 2 years or more and at a temperature of about 5° C. In some embodiments, a stable DP is defined as having a protein concentration of about 1.8 mg/mL to about 2.2 mg/mL after DP storage for about 12 months or more and at a temperature of about 25° C., and/or after storage for about 2 years or more and at a temperature of about 5° C. In some embodiments, a stable DP is defined as having a protein concentration of about 1.85 mg/mL to about 2.15 mg/mL after DP storage for about 12 months or more and at a temperature of about 25° C., and/or after storage for about 2 years or more and at a temperature of about 5° C. In some embodiments, a stable DP is defined as having a protein concentration of about 1.9 mg/mL to 2.1 mg/mL after DP storage for about 12 months or more and at a temperature of about 25° C., and/or after storage for about 2 years or more and at a temperature of about 5° C.

40 mg/mL DP Formulation

In some embodiments, a stable DP is defined as having a protein concentration of about: 30 mg/mL, 31 mg/mL, 32 mg/mL, 33 mg/mL, 34 mg/mL 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, or 50 mg/mL or any value or range in between, after DP storage for about 12 months or more and at a temperature of about 25° C., and/or after storage for about 2 years or more and at a temperature of about 5° C. In some embodiments, a stable DP is defined as having a protein concentration of about 34 mg/mL to about 44 mg/mL after DP storage for about 12 months or more and at a temperature of about 25° C., and/or after storage for about 2 years or more and at a temperature of about 5° C. In some embodiments, a stable DP is defined as having a protein concentration of about 37 mg/mL to about 43 mg/mL after DP storage for about 12 months or more and at a temperature of about 25° C., and/or after storage for about 2 years or more and at a temperature of about 5° C. In some embodiments, a stable DP is defined as having a protein concentration of about 38 mg/mL to 42 mg/mL after DP storage for about 12 months or more and at a temperature of about 25° C., and/or after storage for about 2 years or more and at a temperature of about 5° C.

### Peptide Mapping

Post-translational modifications (PTMs), such as oxidation, deamidation, and isomerization, are enzymatic modifications that may be detected in the structure of an antibody. In some embodiments, the DP stability is assessed based on level of PTMs in the antibody. Test articles are enzymatically digested to yield peptide segments. These peptides are then evaluated by for instance by mass spectrometry (MS), by tandem mass spectrometry (MS-MS) or Ultra High-Performance Liquid Chromatography Mass Spectroscopy (UPLC-MS). Each analyzed peptide sequence is identified relative to its known location within the overall antibody structure. Post-translational modifications are determined by comparing the measured mass of the identified peptide sequence with its expected mass.

### Drug Product Potency

In vitro T-cell activation assay allows assessing the level of DP stability. This activation can be assessed by using, but not limited to, a Nuclear factor of activated T cells-Response Element (NFAT-RE)-mediated luminescence assay.

### GPRC5D/CD3 T-cell Activation Activity

In some embodiments, a stable DP is defined as having a GPRC5D/CD3-mediated T-cell activation activity, relative to a reference, of about: 40%, 50%, 60%, 70%, 80%, 90%, 100%, 110%, 120%, 130%, 140%, 150%, or 160% or any value or range in between after DP storage for about 12 months or more and at a temperature of about 5° C., after storage for about 12 months or more and at a temperature of about 25° C., and/or after storage for about 2 years or more and at a temperature of about 5° C. In some embodiments, a stable DP is defined as having an GPRC5D/CD3-mediated T-cell activation activity ranging from about 50% to about 150% relative to a reference after DP storage for about 12 months or more and at a temperature of about 25° C., and/or after storage for about 2 years or more and at a temperature of about 5° C. In some embodiments, a stable DP is defined as having an GPRC5D/CD3-mediated T-cell activation activity ranging from about 60% to about 140% relative to a reference after DP storage for about 12 months or more and at a temperature of about 25° C., and/or after storage for about 2 years or more and at a temperature of about 5° C. In some embodiments, a stable DP stability is defined as having an GPRC5D/CD3-mediated T-cell activation activity ranging from about 80% to about 120% relative to a reference after DP storage for about 12 months or more and at a temperature of about 25° C., and/or after storage for about 2 years or more and at a temperature of about 5° C. In some embodiments, the GPRC5D/CD3-mediated T-cell activation activity is a talquetamab mediated T-cell activation activity.

### Polysorbate 20 (PS20)

In some embodiments, a stable DP is defined as having a PS20 concentration in percentage weight to volume of about: 0.005%, 0.01%, 0.02%, 0.03%, 0.04%, 0.05%, 0.06%, 0.07%, 0.08%, 0.09%, 0.10%, or any value or range in between after DP storage for about 12 months or more and at a temperature of about 5° C., after storage for about 12 months or more and at a temperature of about 25° C., and/or after storage for about 2 years or more and at a temperature of about 5° C. In some embodiments, a stable DP is defined as having a PS20 concentration of about 0.02% (w/v) to about 0.1% (w/v) after DP storage for about 12 months or more and at a temperature of about 25° C., and/or after storage for about 2 years or more and at a temperature of about 5° C. In one embodiment, a stable DP is defined as having a PS20 concentration of about 0.01% (w/v) to about 0.07% (w/v) after DP storage for about 12 months or more and at a temperature of about 5° C., after storage for about 12 months or more and at a temperature of about 25° C., and/or after storage for about 2 years or more and at a temperature of about 5° C. In some embodiments, a stable DP is defined as having a PS20 concentration of about 0.02% (w/v) to about 0.06% (w/v) after DP storage for about 12 months or more and at a temperature of about 25° C., and/or after storage for about 2 years or more and at a temperature of about 5° C. In some embodiments, a stable DP is defined as having a PS20 concentration of about 0.03% (w/v) to about 0.05% (w/v) after DP storage for about 12 months or more and at a temperature of about 25° C., and/or after storage for about 2 years or more and at a temperature of about 5° C.

### Total Volume of the Aqueous Pharmaceutical Composition

2 mg/mL Formulation

In some embodiments, the total volume of the aqueous pharmaceutical composition (or DP) ranges from about 5 mL to about 10 mL. In some embodiments, the total volume of the aqueous pharmaceutical composition (or DP) ranges from about 0.5 mL to about 20 mL, from about 1 mL to about 15 mL, from about 5 mL to about 10 mL, or from about 6 mL to about 8 mL or any value or range in-between.

In some embodiments, the total volume of the aqueous pharmaceutical composition (or DP) is from about 0.5 mL to about 30 mL. In some embodiments, the total volume of the aqueous pharmaceutical composition (or DP) is from about 0.6 mL to about 30 mL. In some embodiments, the total volume of the aqueous pharmaceutical composition (or DP) is from about 0.7 mL to about 30 mL. In some embodiments, the total volume of the aqueous pharmaceutical composition (or DP) is from about 0.8 mL to about 30 mL. In some embodiments, the total volume of the aqueous pharmaceutical composition (or DP) is from about 0.9 mL to about 30 mL. In some embodiments, the total volume of the aqueous pharmaceutical composition (or DP) is from about 1 mL to about 30 mL. In some embodiments, the total volume of the aqueous pharmaceutical composition (or DP) is from about 2 mL to about 30 mL. In some embodiments, the total volume of the aqueous pharmaceutical composition (or DP) is from about 3 mL to about 30 mL. In some embodiments, the total volume of the aqueous pharmaceutical composition (or DP) is from about 4 mL to about 30 mL. In some embodiments, the total volume of the aqueous pharmaceutical composition (or DP) is from about 5 mL to about 30 mL. In some embodiments, the total volume of the aqueous pharmaceutical composition (or DP) is from about 6 mL to about 30 mL. In some embodiments, the total volume of the aqueous pharmaceutical composition (or DP) is from about 7 mL to about 30 mL. In some embodiments, the total volume of the aqueous pharmaceutical composition (or DP) is from about 8 mL to about 30 mL. In some embodiments, the total volume of the aqueous pharmaceutical composition (or DP) is from about 9 mL to about 30 mL. In some embodiments, the total volume of the aqueous pharmaceutical composition (or DP) is from about 10 mL to about 30 mL. In some embodiments, the total volume of the aqueous pharmaceutical composition (or DP) is from about 11 mL to about 30 mL. In some embodiments, the total volume of the aqueous pharmaceutical composition (or DP) is from about 12 mL to about 30 mL. In some embodiments, the total volume of the aqueous pharmaceutical composition (or DP) is from about 13 mL to about 30 mL. In some embodiments, the total volume of the aqueous pharmaceutical composition (or DP) is from about 14 mL to about 30 mL. In some embodiments, the total volume of the aqueous pharmaceutical composition (or DP) is from about 15 mL to about 30 mL. In some embodiments, the total volume of the aqueous pharmaceutical composition (or DP) is from about 16 mL to about 30 mL. In some embodiments, the total volume of the aqueous pharmaceutical composition (or DP) is from about 17 mL to about 30 mL. In some embodiments, the total volume of the aqueous pharmaceutical composition (or DP) is from about 18 mL to about 30 mL. In some embodiments, the total volume of the aqueous pharmaceutical composition (or DP) is from about 19 mL to about 30 mL. In some embodiments, the total volume of the aqueous pharmaceutical composition (or DP) is from about 20 mL to about 30 mL. In some embodiments, the total volume of the aqueous pharmaceutical composition (or DP) is from about 25 mL to about 30 mL.

In some embodiments, the total volume of the aqueous pharmaceutical composition (or DP) is from about 0.5 mL to about 25 mL. In some embodiments, the total volume of the aqueous pharmaceutical composition (or DP) is from about 0.5 mL to about 20 mL. In some embodiments, the total volume of the aqueous pharmaceutical composition (or DP) is from about 0.5 mL to about 19 mL. In some embodiments, the total volume of the aqueous pharmaceutical composition (or DP) is from about 0.5 mL to about 18 mL. In some embodiments, the total volume of the aqueous pharmaceutical composition (or DP) is from about 0.5 mL to about 17 mL. In some embodiments, the total volume of the aqueous pharmaceutical composition (or DP) is from about 0.5 mL to about 16 mL. In some embodiments, the total volume of the aqueous pharmaceutical composition (or DP) is from about 0.5 mL to about 15 mL. In some embodiments, the total volume of the aqueous pharmaceutical composition (or DP) is from about 0.5 mL to about 14 mL. In some embodiments, the total volume of the aqueous pharmaceutical composition (or DP) is from about 0.5 mL to about 13 mL. In some embodiments, the total volume of the aqueous pharmaceutical composition (or DP) is from about 0.5 mL to about 12 mL. In some embodiments, the total volume of the aqueous pharmaceutical composition (or DP) is from about 0.5 mL to about 11 mL. In some embodiments, the total volume of the aqueous pharmaceutical composition (or DP) is from about 0.5 mL to about 10 mL. In some embodiments, the total volume of the aqueous pharmaceutical composition (or DP) is from about 0.5 mL to about 9 mL. In some embodiments, the total volume of the aqueous pharmaceutical composition (or DP) is from about 0.5 mL to about 8 mL. In some embodiments, the total volume of the aqueous pharmaceutical composition (or DP) is from about 0.5 mL to about 7 mL. In some embodiments, the total volume of the aqueous pharmaceutical composition (or DP) is from about 0.5 mL to about 6 mL. In some embodiments, the total volume of the aqueous pharmaceutical composition (or DP) is from about 0.5 mL to about 5 mL. In some embodiments, the total volume of the aqueous pharmaceutical composition (or DP) is from about 0.5 mL to about 4 mL. In some embodiments, the total volume of the aqueous pharmaceutical composition (or DP) is from about 0.5 mL to about 3 mL. In some embodiments, the total volume of the aqueous pharmaceutical composition (or DP) is from about 0.5 mL to about 2 mL. In some embodiments, the total volume of the aqueous pharmaceutical composition (or DP) is from about 0.5 mL to about 1 mL. In some embodiments, the total volume of the aqueous pharmaceutical composition (or DP) is from about 0.5 mL to about 0.9 mL. In some embodiments, the total volume of the aqueous pharmaceutical composition (or DP) is from about 0.5 mL to about 0.8 mL. In some embodiments, the total volume of the aqueous pharmaceutical composition (or DP) is from about 0.5 mL to about 0.7 mL. In some embodiments, the total volume of the aqueous pharmaceutical composition (or DP) is from about 0.5 mL to about 0.6 mL.

In some embodiments, the total volume of the aqueous pharmaceutical composition is about: 0.5 mL, 0.6 mL, 0.7 mL, 0.8 mL, 0.9 mL, 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 18 mL, 19 mL, 20 mL, 25 mL, or 30 mL or any value or range in between. In some embodiments, the total volume of the aqueous pharmaceutical composition is 0.5 mL. In some embodiments, the total volume of the aqueous pharmaceutical composition is 0.6 mL. In some embodiments, the total volume of the aqueous pharmaceutical composition is 0.7 mL. In some embodiments, the total volume of the aqueous pharmaceutical composition is 0.8 mL. In some embodiments, the total volume of the aqueous pharmaceutical composition is 0.9 mL. In some embodiments, the total volume of the aqueous pharmaceutical composition is 1 mL. In some embodiments, the total volume of the aqueous pharmaceutical composition is 2 mL. In some embodiments, the total volume of the aqueous pharmaceutical composition is 3 mL. In some embodiments, the total volume of the aqueous pharmaceutical composition is 4 mL. In some embodiments, the total volume of the aqueous pharmaceutical composition is 5 mL. In some embodiments, the total volume of the aqueous pharmaceutical composition is 6 mL. In some embodiments, the total volume of the aqueous pharmaceutical composition is 7 mL. In some embodiments, the total volume of the aqueous pharmaceutical composition is 8 mL. In some embodiments, the total volume of the aqueous pharmaceutical composition is 9 mL. In some embodiments, the total volume of the aqueous pharmaceutical composition is 10 mL. In some embodiments, the total volume of the aqueous pharmaceutical composition is 11 mL. In some embodiments, the total volume of the aqueous pharmaceutical composition is 12 mL. In some embodiments, the total volume of the aqueous pharmaceutical composition is 13 mL. In some embodiments, the total volume of the aqueous pharmaceutical composition is 14 mL. In some embodiments, the total volume of the aqueous pharmaceutical composition is 15 mL. In some embodiments, the total volume of the aqueous pharmaceutical composition is 16 mL. In some embodiments, the total volume of the aqueous pharmaceutical composition is 17 mL. In some embodiments, the total volume of the aqueous pharmaceutical composition is 18 mL. In some embodiments, the total volume of the aqueous pharmaceutical composition is 19 mL. In some embodiments, the total volume of the aqueous pharmaceutical composition is 20 mL. In some embodiments, the total volume of the aqueous pharmaceutical composition is 25 mL. In some embodiments, the total volume of the aqueous pharmaceutical composition is 30 mL. In some embodiments, the aqueous pharmaceutical composition is a stable aqueous pharmaceutical composition. In some embodiments, the stability of the aqueous pharmaceutical composition is as defined and provided for herein.

40 mg/mL Formulation

In some embodiments, the total volume of the aqueous pharmaceutical composition (or DP) ranges from about 5 mL to about 10 mL. In some embodiments, the total volume of the aqueous pharmaceutical composition (or DP) ranges from about 0.5 mL to about 20 mL, from about 1 mL to about 15 mL, from about 5 mL to about 10 mL, or from about 6 mL to about 8 mL, or any value or range in between.

In some embodiments, the total volume of the aqueous pharmaceutical composition (or DP) is from about 0.5 mL to about 30 mL. In some embodiments, the total volume of the aqueous pharmaceutical composition (or DP) is from about 0.6 mL to about 30 mL. In some embodiments, the total volume of the aqueous pharmaceutical composition (or DP) is from about 0.7 mL to about 30 mL. In some embodiments, the total volume of the aqueous pharmaceutical composition (or DP) is from about 0.8 mL to about 30 mL. In some embodiments, the total volume of the aqueous pharmaceutical composition (or DP) is from about 0.9 mL to about 30 mL. In some embodiments, the total volume of the aqueous pharmaceutical composition (or DP) is from about 1 mL to about 30 mL. In some embodiments, the total volume of the aqueous pharmaceutical composition (or DP) is from about 2 mL to about 30 mL. In some embodiments, the total volume of the aqueous pharmaceutical composition (or DP) is from about 3 mL to about 30 mL. In some embodiments, the total volume of the aqueous pharmaceutical composition (or DP) is from about 4 mL to about 30 mL. In some embodiments, the total volume of the aqueous pharmaceutical composition (or DP) is from about 5 mL to about 30 mL. In some embodiments, the total volume of the aqueous pharmaceutical composition (or DP) is from about 6 mL to about 30 mL. In some embodiments, the total volume of the aqueous pharmaceutical composition (or DP) is from about 7 mL to about 30 mL. In some embodiments, the total volume of the aqueous pharmaceutical composition (or DP) is from about 8 mL to about 30 mL. In some embodiments, the total volume of the aqueous pharmaceutical composition (or DP) is from about 9 mL to about 30 mL. In some embodiments, the total volume of the aqueous pharmaceutical composition (or DP) is from about 10 mL to about 30 mL. In some embodiments, the total volume of the aqueous pharmaceutical composition (or DP) is from about 11 mL to about 30 mL. In some embodiments, the total volume of the aqueous pharmaceutical composition (or DP) is from about 12 mL to about 30 mL. In some embodiments, the total volume of the aqueous pharmaceutical composition (or DP) is from about 13 mL to about 30 mL. In some embodiments, the total volume of the aqueous pharmaceutical composition (or DP) is from about 14 mL to about 30 mL. In some embodiments, the total volume of the aqueous pharmaceutical composition (or DP) is from about 15 mL to about 30 mL. In some embodiments, the total volume of the aqueous pharmaceutical composition (or DP) is from about 16 mL to about 30 mL. In some embodiments, the total volume of the aqueous pharmaceutical composition (or DP) is from about 17 mL to about 30 mL. In some embodiments, the total volume of the aqueous pharmaceutical composition (or DP) is from about 18 mL to about 30 mL. In some embodiments, the total volume of the aqueous pharmaceutical composition (or DP) is from about 19 mL to about 30 mL. In some embodiments, the total volume of the aqueous pharmaceutical composition (or DP) is from about 20 mL to about 30 mL. In some embodiments, the total volume of the aqueous pharmaceutical composition (or DP) is from about 25 mL to about 30 mL.

In some embodiments, the total volume of the aqueous pharmaceutical composition (or DP) is from about 0.5 mL to about 25 mL. In some embodiments, the total volume of the aqueous pharmaceutical composition (or DP) is from about 0.5 mL to about 20 mL. In some embodiments, the total volume of the aqueous pharmaceutical composition (or DP) is from about 0.5 mL to about 19 mL. In some embodiments, the total volume of the aqueous pharmaceutical composition (or DP) is from about 0.5 mL to about 18 mL. In some embodiments, the total volume of the aqueous pharmaceutical composition (or DP) is from about 0.5 mL to about 17 mL. In some embodiments, the total volume of the aqueous pharmaceutical composition (or DP) is from about 0.5 mL to about 16 mL. In some embodiments, the total volume of the aqueous pharmaceutical composition (or DP) is from about 0.5 mL to about 15 mL. In some embodiments, the total volume of the aqueous pharmaceutical composition (or DP) is from about 0.5 mL to about 14 mL. In some embodiments, the total volume of the aqueous pharmaceutical composition (or DP) is from about 0.5 mL to about 13 mL. In some embodiments, the total volume of the aqueous pharmaceutical composition (or DP) is from about 0.5 mL to about 12 mL. In some embodiments, the total volume of the aqueous pharmaceutical composition (or DP) is from about 0.5 mL to about 11 mL. In some embodiments, the total volume of the aqueous pharmaceutical composition (or DP) is from about 0.5 mL to about 10 mL. In some embodiments, the total volume of the aqueous pharmaceutical composition (or DP) is from about 0.5 mL to about 9 mL. In some embodiments, the total volume of the aqueous pharmaceutical composition (or DP) is from about 0.5 mL to about 8 mL. In some embodiments, the total volume of the aqueous pharmaceutical composition (or DP) is from about 0.5 mL to about 7 mL. In some embodiments, the total volume of the aqueous pharmaceutical composition (or DP) is from about 0.5 mL to about 6 mL. In some embodiments, the total volume of the aqueous pharmaceutical composition (or DP) is from about 0.5 mL to about 5 mL. In some embodiments, the total volume of the aqueous pharmaceutical composition (or DP) is from about 0.5 mL to about 4 mL. In some embodiments, the total volume of the aqueous pharmaceutical composition (or DP) is from about 0.5 mL to about 3 mL. In some embodiments, the total volume of the aqueous pharmaceutical composition (or DP) is from about 0.5 mL to about 2 mL. In some embodiments, the total volume of the aqueous pharmaceutical composition (or DP) is from about 0.5 mL to about 1 mL. In some embodiments, the total volume of the aqueous pharmaceutical composition (or DP) is from about 0.5 mL to about 0.9 mL. In some embodiments, the total volume of the aqueous pharmaceutical composition (or DP) is from about 0.5 mL to about 0.8 mL. In some embodiments, the total volume of the aqueous pharmaceutical composition (or DP) is from about 0.5 mL to about 0.7 mL. In some embodiments, the total volume of the aqueous pharmaceutical composition (or DP) is from about 0.5 mL to about 0.6 mL.

In some embodiments, the total volume of the aqueous pharmaceutical composition is about: 0.5 mL, 0.6 mL, 0.7 mL, 0.8 mL, 0.9 mL, 1 mL, 1.1 mL, 1.2 mL, 1.3 mL, 1.4 mL, 1.5 mL, 2 mL, 3 mL, 4 mL, 5 mL, 6 mL, 8 mL, 9 mL, 10 mL, 11 mL, 12 mL, 13 mL, 14 mL, 15 mL, 16 mL, 18 mL, 19 mL, 20 mL, 25 mL, or 30 mL or any value or range in between. In some embodiments, the total volume of the aqueous pharmaceutical composition is 0.5 mL. In some embodiments, the total volume of the aqueous pharmaceutical composition is 0.6 mL. In some embodiments, the total volume of the aqueous pharmaceutical composition is 0.7 mL. In some embodiments, the total volume of the aqueous pharmaceutical composition is 0.8 mL. In some embodiments, the total volume of the aqueous pharmaceutical composition is 0.9 mL. In some embodiments, the total volume of the aqueous pharmaceutical composition is 1 mL. In some embodiments, the total volume of the aqueous pharmaceutical composition is 1.1 mL. In some embodiments, the total volume of the aqueous pharmaceutical composition is 1.2 mL. In some embodiments, the total volume of the aqueous pharmaceutical composition is 1.3 mL. In some embodiments, the total volume of the aqueous pharmaceutical composition is 1.4 mL. In some embodiments, the total volume of the aqueous pharmaceutical composition is 1.5 mL. In some embodiments, the total volume of the aqueous pharmaceutical composition is 2 mL. In some embodiments, the total volume of the aqueous pharmaceutical composition is 3 mL. In some embodiments, the total volume of the aqueous pharmaceutical composition is 4 mL. In some embodiments, the total volume of the aqueous pharmaceutical composition is 5 mL. In some embodiments, the total volume of the aqueous pharmaceutical composition is 6 mL. In some embodiments, the total volume of the aqueous pharmaceutical composition is 7 mL. In some embodiments, the total volume of the aqueous pharmaceutical composition is 8 mL. In some embodiments, the total volume of the aqueous pharmaceutical composition is 9 mL. In some embodiments, the total volume of the aqueous pharmaceutical composition is 10 mL. In some embodiments, the total volume of the aqueous pharmaceutical composition is 11 mL. In some embodiments, the total volume of the aqueous pharmaceutical composition is 12 mL. In some embodiments, the total volume of the aqueous pharmaceutical composition is 13 mL. In some embodiments, the total volume of the aqueous pharmaceutical composition is 14 mL. In some embodiments, the total volume of the aqueous pharmaceutical composition is 15 mL. In some embodiments, the total volume of the aqueous pharmaceutical composition is 16 mL. In some embodiments, the total volume of the aqueous pharmaceutical composition is 17 mL. In some embodiments, the total volume of the aqueous pharmaceutical composition is 18 mL. In some embodiments, the total volume of the aqueous pharmaceutical composition is 19 mL. In some embodiments, the total volume of the aqueous pharmaceutical composition is 20 mL. In some embodiments, the total volume of the aqueous pharmaceutical composition is 25 mL. In some embodiments, the total volume of the aqueous pharmaceutical composition is 30 mL. In some embodiments, the aqueous pharmaceutical composition is a stable aqueous pharmaceutical composition. In some embodiments, the stability of the aqueous pharmaceutical composition is as defined and provided for herein.

### Methods

In some embodiments, methods of treating cancer in a subject in need thereof are provided. In some embodiments, the method comprises administering to the subject an aqueous pharmaceutical composition as disclosed herein. In some embodiments, the aqueous pharmaceutical composition is a stable aqueous pharmaceutical composition. In some embodiments, the stable aqueous pharmaceutical composition is as defined and provided for herein. In some embodiments, the administration is subcutaneous.

### Cancers

In some embodiments, the cancer is a hematological malignancy.

In some embodiments, the hematological malignancy is multiple myeloma. In some embodiments, the subject has a newly diagnosed multiple myeloma. In some embodiments, the subject is relapsed or refractory to treatment with a prior anti-cancer therapeutic, such as a therapeutic used to treat multiple myeloma or other hematological malignancies.

In some embodiments, the subject is refractory or relapsed to treatment with one or more treatments or therapies, such as THALOMID® (thalidomide), REVLIMID® (lenalidomide), POMALYST® (pomalidomide), VELCADE® (bortezomib), NINLARO (ixazomib), KYPROLIS® (carfilzomib), FARADYK® (panobinostat), AREDIA® (pamidronate), ZOMETA® (zoledronic acid), DARZALEX® (daratumumab), elotozumab or melphalan, Xpovio® (Selinexor), Venclexta® (Venetoclax), GSK 916, CAR-T therapies, BCMA-directed therapies, or other GPRC5D-directed therapies.

Various qualitative and/or quantitative methods can be used to determine relapse or refractory nature of the disease. Symptoms that can be associated are for example a decline or plateau of the well-being of the patient or re-establishment or worsening of various symptoms associated with solid tumors, and/or the spread of cancerous cells in the body from one location to other organs, tissues or cells.

In some embodiments, the multiple myeloma is relapsed or refractory to treatment with at least four prior therapies, including a proteasome inhibitor, an immunomodulatory agent and an anti-CD38 monoclonal antibody, e.g., selected from daratumumab, selinexor, lenalinomide, bortezomib, pomalidomide, carfilzomib, or thalidomide, or any combination thereof. In some embodiments, the multiple myeloma is relapsed or refractory to treatment with at least 3 prior therapies, including an immunomodulatory agent, a proteasome inhibitor, and an anti-CD38 antibody, e.g., selected from daratumumab, selinexor, lenalinomide, bortezomib, pomalidomide, carfilzomib, or thalidomide, or any combination thereof, and the patient demonstrated disease progression or did not respond to the last therapy.

In some embodiments, the multiple myeloma is relapsed or refractory to treatment with an anti-CD38 antibody, selinexor, venetoclax, lenalinomide, bortezomib, pomalidomide, carfilzomib, elotozumab, ixazomib, melphalan or thalidomide, or any combination thereof.

In some embodiments, the multiple myeloma is a high-risk multiple myeloma. Subjects with high-risk multiple myeloma are known to relapse early and have poor prognosis and outcome. Subjects can be classified as having high-risk multiple myeloma is they have one or more of the following cytogenetic abnormalities: t(4;14)(p16;q32), t(14;16)(q32;q23), del17p, lqAmp, t(4;14)(p16;q32) and t(14;16)(q32;q23), t(4;14)(p16;q32) and del17p, t(14;16)(q32;q23) and del17p, or t(4;14)(p16;q32), t(14;16)(q32;q23) and del17p. In some embodiments, the subject having the high-risk multiple myeloma has one or more chromosomal abnormalities comprising: t(4;14)(p16;q32), t(14;16)(q32;q23), del17p, lqAmp, t(4;14)(p16;q32) and t(14;16)(q32;q23), t(4;14)(p16;q32) and del17p, t(14;16)(q32;q23) and del17p; or t(4;14)(p16;q32), t(14;16)(q32;q23) and del17p, or any combination thereof.

The cytogenetic abnormalities can be detected for example by fluorescent in situ hybridization (FISH). In chromosomal translocations, an oncogene is translocated to the IgH region on chromosome 14q32, resulting in dysregulation of these genes. t(4;14)(p16;q32) involves translocation of fibroblast growth factor receptor 3 (FGFR3) and multiple myeloma SET domain containing protein (MMSET) (also called WHSC1/NSD2), and t(14;16)(q32;q23) involves translocation of the MAF transcription factor C-MAF. Deletion of 17p (del17p) involves loss of the p53 gene locus.

Chromosomal rearrangements can be identified using well known methods, for example fluorescent in situ hybridization, karyotyping, pulsed field gel electrophoresis, or sequencing.

Methods of treating multiple myeloma with a GPRC5D×CD3 bispecific antibody, such as talquetamab, are described in WO 2022/058445, which is incorporated by reference herein. A phase 1 clinical study evaluating talquetamab is also described in Chari et al., Talquetamab, a T-Cell-Redirecting GPRC5D Bispecific Antibody for Multiple Myeloma, N Engl J Med 2022; 387:2232-2244, which is incorporated by reference herein.

According to an embodiment, a method of treating a human subject with relapsed or refractory multiple myeloma comprises administering a stable aqueous pharmaceutical composition of the present invention on a dosing schedule according to the subject's body weight, wherein said dosing schedule comprises: subcutaneously administering an aqueous pharmaceutical composition to the subject at step-up doses of 0.01 mg/kg, 0.06 mg/kg, 0.4 mg/kg, and 0.8 mg/kg on days 1, 3, 5, and 7, respectively, followed by subcutaneously administering an aqueous pharmaceutical composition to the subject at a treatment dose of 0.8 mg/kg once every two weeks, wherein the aqueous pharmaceutical composition comprising 2 mg/mL of talquetamab is administered for the doses of 0.01 mg/kg and 0.06 mg/kg and the aqueous pharmaceutical composition comprising 40 mg/mL of talquetamab is administered for each dose of 0.4 mg/kg and 0.8 mg/kg. As used herein with respect to dosing schedules, mg/kg refers to mg antibody, such as talquetamab, per kg of the subject's body weight.

According to an alternative embodiment, a method of treating a human subject with relapsed or refractory multiple myeloma comprises administering a stable aqueous pharmaceutical composition of the present invention on a dosing schedule according to the subject's body weight, wherein said dosing schedule comprises: subcutaneously administering an aqueous pharmaceutical composition to the subject at step-up doses of 0.01 mg/kg, 0.06 mg/kg, 0.3 mg/kg, and 0.8 mg/kg on days 1, 3, 5, and 7, respectively, followed by subcutaneously administering an aqueous pharmaceutical composition to the subject at a treatment dose of 0.8 mg/kg once every two weeks, wherein the aqueous pharmaceutical composition comprising 2 mg/mL of talquetamab is administered for the doses of 0.01 mg/kg and 0.06 mg/kg and the aqueous pharmaceutical composition comprising 40 mg/mL of talquetamab is administered for each dose of 0.3 mg/kg and 0.8 mg/kg.

According to an alternative embodiment, a method of treating a human subject with relapsed or refractory multiple myeloma comprises administering a stable aqueous pharmaceutical composition of the present invention on a dosing schedule according to the subject's body weight, wherein said dosing schedule comprises: subcutaneously administering an aqueous pharmaceutical composition to the subject at step-up doses of 0.01 mg/kg, 0.06 mg/kg and 0.4 mg/kg, on days 1, 3 and 5, respectively, followed by subcutaneously administering an aqueous pharmaceutical composition to the subject at a treatment dose of 0.4 mg/kg once per week, wherein the aqueous pharmaceutical composition comprising 2 mg/mL of talquetamab is administered for the doses of 0.01 mg/kg and 0.06 mg/kg and the aqueous pharmaceutical composition comprising 40 mg/mL of talquetamab is administered for each dose of 0.4 mg/kg.

In preferred embodiments, the pharmaceutical composition is therapeutically effective in treating relapsed or refractory multiple myeloma in a human subject, in particular, the composition is effective in eliciting a partial response, very good partial response, complete response or stringent complete response, as determined by International Myeloma Working Group (TMWG) response criteria. As used herein, overall response rate (ORR) refers to the percentage ofpatients in a population that achieve a partial response (PR) or better, i.e., a partial response, very good partial response, complete response or stringent complete response. IMWG criteria for response to Multiple Myeloma treatment are provided in Table A below.

TABLE A

| Response | Response Criteria |
|---|---|
| sCR = stringent complete | □ CR as defined below, plus |
| response | □ Normal FLC ratio, and |
| | □ Absence of clonal PCs by immunohistochemistry, |
| | immunofluorescence^{a} or 2- to 4-color flow cytometry |
| CR = complete | □ Negative immunofixation on the serum and urine, and |
| response | □ Disappearance of any soft tissue plasmacytomas, and |
| | □ <5% PCs in bone marrow |
| VGPR = very good | □ Serum and urine M-component detectable by immunofixation |
| partial response | but not on electrophoresis, or |
| | □ ≥90% reduction in serum M-protein plus urine M-protein < |
| | 100 mg/24 hours |
| PR = partial | □ ≥50% reduction of serum M-protein and reduction in 24-hour |
| response | urinary M-protein by ≥90% or to <200 mg/24 hours |
| | □ If the serum and urine M-protein are not measurable, a decrease |
| | of ≥50% in the difference between involved and uninvolved FLC |
| | levels is required in place of the M-protein criteria |
| | □ If serum and urine M-protein are not measurable, and serum |
| | free light assay is also not measurable, ≥50% reduction in bone |
| | marrow PCs is required in place of M-protein, |
| | provided baseline bone marrow plasma cell percentage was ≥30% |
| | □ In addition to the above criteria, if present at baseline, a ≥50% |
| | reduction in the size of soft tissue plasmacytomas is also required. |

CR = complete response;
FLC = free light chain;
IMWG = International Myeloma Working Group;
M-protein = monoclonal paraprotein;
MR = minimal response;
PC = plasma cell;
PD = progressive disease;
PR = partial response;
sCR = stringent complete response;
SD = stable disease;
VGPR = very good partial response
^{a}Presence/absence of clonal cells is based upon the kappa/lambda ratio. An abnormal kappa/lambda ratio by immunohistochemistry or immunofluorescence requires a minimum of 100 plasma cells for analysis. An abnormal ratio reflecting presence of an abnormal clone is kappa/lambda of >4:1 or <1:2.
* Clarifications to IMWG criteria for coding CR and VGPR in subjects in whom the only measurable disease is by serum FLC levels: CR in such subjects indicates a normal FLC ratio of 0.26 to 1.65 in addition to CR criteria listed above. VGPR in such subjects requires a >90% decrease in the difference between involved and uninvolved FLC levels.

IMWG criteria for response to Multiple Myeloma treatment are also described, for example, in Durie et al., Kumar et al. and Rajkumar et al., which are incorporated by reference herein: Durie B G, Harousseau J L, Miguel J S, et al. International uniform response criteria for multiple myeloma. Leukemia. 2006; 20(9):1467-1473; Kumar S, Paiva B, Anderson K C, et al. International Myeloma Working Group consensus criteria for response and minimal residual disease assessment in multiple myeloma. Lancet Oncol. 2016;17(8):e328-346; Rajkumar S V, Harousseau J L, Durie B, et al. Consensus recommendations for the uniform reporting of clinical Trials: report of the International Myeloma Workshop Consensus Panel 1. Blood. 2011; 117(18):4691-4695.

In certain embodiments, pharmaceutical compositions of the present invention elicit an overall response rate (ORR) of at least 70% in a population of patients as defined by IMWG criteria when subcutaneously administered to each subject according to a dosing schedule shown in Table B, C or D:

TABLE B

| Phase | Day | Talquetamab Dose^{a} |
|---|---|---|
| Step-up Phase | Day 1 | 0.01 mg/kg |
| | Day 3^{b} | 0.06 mg/kg |
| | Day 5^{b} | 0.4 mg/kg |
| Treatment Phase | Once a week thereafter^{c} | 0.4 mg/kg |

^{a}Based on actual body weight.
^{b}Dose may be administered between 2 to 4 days after the previous dose and may be given up to 7 days after the previous dose to allow for resolution of adverse reactions.
^{c}Maintain a minimum of 6 days between weekly doses and a minimum of 12 days between biweekly (every 2 weeks) doses.

TABLE C

| Phase | Day | Talquetamab Dosea |
|---|---|---|
| Step-up Phase | Day 1 | 0.01 mg/kg |
| | Day 3^{b} | 0.06 mg/kg |
| | Day 5^{b} | 0.4 mg/kg |
| | Day 7^{b} | 0.8 mg/kg |
| Treatment Phase | Once every 2 weeks thereafter^{c} | 0.8 mg/kg |

^{a}Based on actual body weight.
^{b}Dose may be administered between 2 to 4 days after the previous dose and may be given up to 7 days after the previous dose to allow for resolution of adverse reactions.
^{c}Maintain a minimum of 6 days between weekly doses and a minimum of 12 days between biweekly (every 2 weeks) doses.

TABLE D

| Phase | Day | Talquetamab Dose^{a} |
|---|---|---|
| Step-up Phase | Day 1 | 0.01 mg/kg |
| | Day 3^{b} | 0.06 mg/kg |
| | Day 5^{b} | 0.3 mg/kg |
| | Day 7^{b} | 0.8 mg/kg |
| Treatment Phase | Once every 2 weeks thereafter^{c} | 0.8 mg/kg |

^{a}Based on actual body weight.
^{b}Dose may be administered between 2 to 4 days after the previous dose and may be given up to 7 days after the previous dose to allow for resolution of adverse reactions.
^{c}Maintain a minimum of 6 days between weekly doses and a minimum of 12 days between biweekly (every 2 weeks) doses.

In certain embodiments, the aqueous pharmaceutical composition comprising 2 mg/mL of talquetamab (administered for doses of 0.01 mg/kg and 0.06 mg/kg in Tables B-D) has the composition shown in Table 26 or Table 27 and the aqueous pharmaceutical composition comprising 40 mg/mL of talquetamab (administered for doses of 0.3 mg/kg, 0.4 mg/kg and 0.8 mg/kg in Tables B-D) has the composition shown in Table 22 or Table 23.

In certain embodiments, the aqueous pharmaceutical composition comprising 2 mg/mL of talquetamab, used for doses of 0.01 mg/kg and 0.06 mg/kg, has the following composition per 1.5 mL: 3 mg of talquetamab, edetate disodium (0.027 mg), glacial acetic acid (0.36 mg), polysorbate 20 (0.6 mg), sodium acetate (1.39 mg), sucrose (120 mg), and Water for Injection, USP.

In certain embodiments, the aqueous pharmaceutical composition comprising 40 mg/mL of talquetamab used for doses of 0.3 mg/kg, 0.4 mg/kg and 0.8 mg/kg has the following composition per 1 mL: 40 mg of talquetamab, edetate disodium (0.018 mg), glacial acetic acid (0.24 mg), polysorbate 20 (0.4 mg), sodium acetate (0.90 mg), sucrose (80 mg), and Water for Injection, USP.

According to certain embodiments, the method of treatment further comprises administering pretreatment medications 1-3 hours before each step-up dose, wherein said pretreatment medications comprise: Corticosteroid (oral or intravenous dexamethasone, 16 mg or equivalent); Antihistamines (oral or intravenous diphenhydramine, 50 mg or equivalent); and Antipyretics (oral or intravenous acetaminophen, 650 mg to 1000 mg or equivalent).

According to certain embodiments, methods of treatment described herein are effective in eliciting a clinical response in the subject according to International Myeloma Working Group (IMWG) diagnostic criteria. In certain embodiments, the methods result in an overall response rate (ORR) of at least 50%, at least 55%, at least 60%, at least 65%, or at least 66%, or at least 67%, or at least 68%, or at least 69%, or at least 70%, or at least 71%, or at least 72%, or at least 73%, or at least 74% in a population of human subjects, according to IMWG criteria, e.g., as described in Example 14.

Also provided herein is a method for preparing an aqueous pharmaceutical composition of a bispecific G-protein coupled receptor, class C, group 5, member D (GPRC5D) antibody or antigen-binding fragment thereof, the bispecific GPRC5D/CD3 antibody or antigen-binding fragment thereof comprising: a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively; a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively; a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively; the method comprising combining a composition comprising about 2 mg/mL of the bispecific GPRC5D/CD3 antibody, about 15 mM of acetate and/or a pharmaceutically acceptable acetate salt, about 8% (w/v) sucrose, about 18 μg/mL of EDTA, and about 0.04% polysorbate (PS) 20, wherein the stable aqueous pharmaceutical composition has a pH of about 5.2. In some embodiments, the aqueous pharmaceutical composition is a stable aqueous pharmaceutical composition, wherein the stability of the aqueous pharmaceutical composition is as defined and provided for herein.

In some embodiments, the bispecific GPRC5D/CD3 antibody comprises a VH1 having the amino acid sequence of SEQ ID NO:7 and a VL1 having the amino acid sequence of SEQ ID NO:8. In some embodiments, the bispecific GPRC5D/CD3 antibody comprises a HC1 having the amino acid sequence of SEQ ID NO:9, and a LC1 having the amino acid sequence of SEQ ID NO:10. In some embodiments, the bispecific GPRC5D/CD3 antibody comprises a VH2 having the amino acid sequence of SEQ ID NO:17, and a VL2 having the amino acid sequence of SEQ ID NO:18. In certain embodiments, the bispecific GPRC5D/CD3 antibody comprises a HC2 having the amino acid sequence of SEQ ID NO:19, and a LC2 having the amino acid sequence of SEQ ID NO:20.

In some embodiments, the bispecific GPRC5D/CD3 antibody can, for example, be talquetamab. In some embodiments, talquetamab comprises a first heavy chain (HC1), a first light chain (LC1), a second heavy chain (HC2), and a second light chain (LC2), wherein the HC1 is associated with LC1 and the HC2 is associated with LC2, wherein HC1 and LC1 form a first antigen-binding site that immunospecifically binds to GPRC5D and wherein HC2 and LC2 form a second antigen-binding site that immunospecifically binds to CD3. In some embodiments, talquetamab comprises a HC1 of SEQ ID NO: 9, a LC1 of SEQ ID NO: 10, a HC2 of SEQ ID NO: 19, and a LC2 of SEQ ID NO: 20. In some embodiments, the GPRC5D arm and the CD3 arm of talquetamab form a functional bispecific antibody through an interaction between their respective Fc domains.

Also provided herein is a method for preparing a stable aqueous pharmaceutical composition of a bispecific G-protein coupled receptor, class C, group 5, member D (GPRC5D) antibody or antigen-binding fragment thereof, the bispecific GPRC5D/CD3 antibody or antigen-binding fragment thereof comprising: a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively; a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively; a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively; the method comprising combining a composition comprising about 40 mg/mL of the bispecific GPRC5D/CD3 antibody, about 15 mM of acetate and/or a pharmaceutically acceptable acetate salt, about 8% (w/v) sucrose, about 18 μg/mL of EDTA, and about 0.04% polysorbate (PS) 20, wherein the stable aqueous pharmaceutical composition has a pH of about 5.2. In some embodiments, the stability of the aqueous pharmaceutical composition is as defined and provided for herein.

In some embodiments, the bispecific GPRC5D/CD3 antibody comprises a VH1 having the amino acid sequence of SEQ ID NO:7 and a VL1 having the amino acid sequence of SEQ ID NO:8. In some embodiments, the bispecific GPRC5D/CD3 antibody comprises a HC1 having the amino acid sequence of SEQ ID NO:9, and a LC1 having the amino acid sequence of SEQ ID NO:10. In some embodiments, the bispecific GPRC5D/CD3 antibody comprises a VH2 having the amino acid sequence of SEQ ID NO:17, and a VL2 having the amino acid sequence of SEQ ID NO:18. In certain embodiments, the bispecific GPRC5D/CD3 antibody comprises a HC2 having the amino acid sequence of SEQ ID NO:19, and a LC2 having the amino acid sequence of SEQ ID NO:20.

In some embodiments, the bispecific GPRC5D/CD3 antibody can, for example, be talquetamab. In some embodiments, talquetamab comprises a first heavy chain (HC1), a first light chain (LC1), a second heavy chain (HC2), and a second light chain (LC2), wherein the HC1 is associated with LC1 and the HC2 is associated with LC2, wherein HC1 and LC1 form a first antigen-binding site that immunospecifically binds to GPRC5D and wherein HC2 and LC2 form a second antigen-binding site that immunospecifically binds to CD3. In some embodiments, talquetamab comprises a HC1 of SEQ ID NO: 9, a LC1 of SEQ ID NO: 10, a HC2 of SEQ ID NO: 19, and a LC2 of SEQ ID NO: 20. In some embodiments, the GPRC5D arm and the CD3 arm of talquetamab form a functional bispecific antibody through an interaction between their respective Fc domains.

The aqueous pharmaceutical compositions disclosed herein can be packaged into kits, containers, packs, dispensers, or vials. In some embodiments, the aqueous pharmaceutical composition is a stable aqueous pharmaceutical composition. In some embodiments, the stability of the aqueous pharmaceutical composition is as defined and provided for herein.

Provided herein is a kit comprising the disclosed aqueous pharmaceutical compositions and instructions for use thereof. In some embodiments, the aqueous pharmaceutical composition is a stable aqueous pharmaceutical composition. In some embodiments, the stability of the aqueous pharmaceutical composition is as defined and provided for herein.

Also provided herein is an article of manufacture comprising a container holding the disclosed aqueous pharmaceutical composition. In some embodiments, the aqueous pharmaceutical composition is a stable aqueous pharmaceutical composition. In some embodiments, the stability of the aqueous pharmaceutical composition is as defined and provided for herein. In some embodiments, the container is a vial with a stopper pierceable by a syringe.

### EMBODIMENTS

Provided here are illustrative embodiments of the disclosed technology. These embodiments are illustrative only and do not limit the scope of the present disclosure or of the claims attached hereto.

In some embodiments, the following embodiments are provided:

1a. An aqueous pharmaceutical composition comprising:
a) a concentration of about 0.5 mg/mL to about 100 mg/mL of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
b) about 10 mM to about 20 mM of acetate and/or pharmaceutically acceptable acetate salt;
c) about 6% (w/v) to about 10% (w/v) of sucrose;
d) about 7 μg/mL to about 33 μg/mL of ethylenediaminetetraacetic acid (EDTA);
e) about 0.01% to about 0.07% polysorbate 20; and
f) a pH from about 4.6 to about 5.8.
   1b. An aqueous pharmaceutical composition comprising:
a) a concentration of about 0.5 mg/mL to about 3.5 mg/mL of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
b) about 10 mM to about 20 mM of acetate and/or pharmaceutically acceptable acetate salt;
c) about 6% (w/v) to about 10% (w/v) of sucrose;
d) about 7 μg/mL to about 33 μg/mL of ethylenediaminetetraacetic acid (EDTA);
e) about 0.01% to about 0.07% polysorbate 20; and
f) a pH from about 4.6 to about 5.8.
   2. An aqueous pharmaceutical composition comprising:
a) a concentration of about 25 mg/mL to about 55 mg/mL of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
b) about 10 mM to about 20 mM of acetate and/or pharmaceutically acceptable acetate salt;
c) about 6% (w/v) to about 10% (w/v) of sucrose;
d) about 7 μg/mL to about 33 μg/mL of ethylenediaminetetraacetic acid (EDTA);
e) about 0.01% to about 0.07% polysorbate 20; and
f) a pH from about 4.6 to about 5.8.
   3. The aqueous pharmaceutical composition of embodiment 1a, 1b or 2, wherein the bispecific GPRC5D/CD3 antibody comprises a VH1 having the amino acid sequence of SEQ ID NO:7, and a VL1 having the amino acid sequence of SEQ ID NO:8.
   4. The aqueous pharmaceutical composition of embodiment 1a, 1b or 2, wherein the bispecific GPRC5D/CD3 antibody comprising a HC1 having the amino acid sequence of SEQ ID NO:9, and a LC1 having the amino acid sequence of SEQ ID NO:10.
   5. The aqueous pharmaceutical composition of embodiment 1a, 1b or 2, wherein the bispecific GPRC5D/CD3 antibody comprises a VH2 having the amino acid sequence of SEQ ID NO:17, and a VL2 having the amino acid sequence of SEQ ID NO:18.
   6. The aqueous pharmaceutical composition of embodiment 1a, 1b or 2, wherein the bispecific GPRC5D/CD3 antibody comprises a HC2 having the amino acid sequence of SEQ ID NO:19, and a LC2 having the amino acid sequence of SEQ ID NO:20.
   7. The aqueous pharmaceutical composition of embodiment 1a, 1b or 2, wherein the bispecific GPRC5D/CD3 antibody is talquetamab.
   8. The aqueous pharmaceutical composition of embodiment 1b, wherein the bispecific GPRC5D/CD3 antibody has a concentration of about 1 mg/mL to about 3 mg/mL.
   9. The aqueous pharmaceutical composition of embodiment 8, wherein the bispecific GPRC5D/CD3 antibody has a concentration of about 1.5 mg/mL to about 2.5 mg/mL.
   10. The aqueous pharmaceutical composition of embodiment 9, wherein the bispecific GPRC5D/CD3 antibody has a concentration of about 2 mg/mL.
   11. The aqueous pharmaceutical composition of embodiment 2, wherein the bispecific GPRC5D/CD3 antibody has a concentration of about 30 mg/mL to about 50 mg/mL.
   12. The aqueous pharmaceutical composition of embodiment 11, wherein the bispecific GPRC5D/CD3 antibody has a concentration of about 35 mg/mL to about 45 mg/mL.
   13. The aqueous pharmaceutical composition of embodiment 12, wherein the bispecific GPRC5D/CD3 antibody has a concentration of about 40 mg/mL.
   14. The aqueous pharmaceutical composition of embodiment 1a, 1b or 2, wherein the composition comprises about 12 mM to about 18 mM of acetate and/or pharmaceutically acceptable acetate salt.
   15. The aqueous pharmaceutical composition of embodiment 1a, 1b or 2, wherein the composition comprises about 14 mM to about 16 mM of acetate and/or pharmaceutically acceptable acetate salt.
   16. The aqueous pharmaceutical composition of embodiment 1a, 1b or 2, wherein the composition comprises about 15 mM of acetate and/or pharmaceutically acceptable acetate salt.
   17. The aqueous pharmaceutical composition of embodiment 1a, 1b or 2, wherein the composition comprises about 7% (w/v) to about 9% (w/v) of sucrose.
   18. The aqueous pharmaceutical composition of embodiment 17, wherein the composition comprises about 8% (w/v) of sucrose.
   19. The aqueous pharmaceutical composition of embodiment 1a, 1b or 2, wherein the composition comprises about 15 μg/mL to about 21 μg/mL of EDTA.
   20. The aqueous pharmaceutical composition of embodiment 1a, 1b or 2, wherein the composition comprises about 18 μg/mL of EDTA.
   21. The aqueous pharmaceutical composition of embodiment 1a, 1b or 2, wherein the composition comprises about 0.02% to about 0.06% of PS-20.
   22. The aqueous pharmaceutical composition of embodiment 21, wherein the composition comprises about 0.03 to about 0.05% of PS-20.
   23. The aqueous pharmaceutical composition of embodiment 22, wherein the composition comprises about 0.04% of PS-20.
   24. The aqueous pharmaceutical composition of embodiment 1a, 1b or 2, wherein the pH is about 4.8 to about 5.6.
   25. The aqueous pharmaceutical composition of embodiment 24, wherein the pH is about 4.9 to about 5.5.
   26. The aqueous pharmaceutical composition of embodiment 25, wherein the pH is about 5.2.
   27. The aqueous pharmaceutical composition of embodiment 1b, wherein the composition comprises 2 mg/mL of the bispecific GPRC5D/CD3 antibody, 15 mM of acetate and/or pharmaceutically acceptable acetate salt, 8% (w/v) sucrose, 18 μg/mL of EDTA, 0.04% PS 20, and a pH of 5.2.
   28. The aqueous pharmaceutical composition of embodiment 2, wherein the composition comprises 40 mg/mL of the bispecific GPRC5D/CD3 antibody, 15 mM of acetate and/or pharmaceutically acceptable acetate salt, 8% (w/v) sucrose, 18 μg/mL of EDTA, 0.04% PS 20, and a pH of 5.2.
   29. The aqueous pharmaceutical composition of any one of embodiments 1a, 1b, or 2-28, wherein the aqueous pharmaceutical composition is stable.
   30. The aqueous pharmaceutical composition of embodiment 29, wherein stability is defined based on color of solution, pH, turbidity, percentage of purity, percentage of new peaks, percentage of main component, percentage of high molecular weight species (HWMS), percentage of low molecular weight species (LMWS), percentage of sum of acidic peaks, percentage of sum of basic peaks, protein concentration, percentage of T cell activation, percentage of PS 20 (w/v), or any combination thereof.
   31. The aqueous pharmaceutical composition of embodiment 29 or 30, wherein the aqueous pharmaceutical composition is stable at a temperature of about 2-8° C. for at least two years.
   32. A method of treating cancer in a human subject in need thereof, the method comprising administering to the subject the aqueous pharmaceutical composition of any one of embodiments 1a, 1b, or 2-31.
   33. The method of embodiment 32, wherein the administering is subcutaneous.
   34. A method for preparing a stable aqueous pharmaceutical composition of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
(1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
   the method comprising combining a composition comprising about 2 mg/mL of the bispecific GPRC5D/CD3 antibody, about 15 mM of acetate and/or a pharmaceutically acceptable acetate salt, about 8% (w/v) sucrose, about 18 μg/mL of EDTA, and about 0.04% polysorbate (PS) 20, wherein the stable aqueous pharmaceutical composition has a pH of about 5.2.
   35. A method for preparing a stable aqueous pharmaceutical composition of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
(1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
   the method comprising combining a composition comprising about 40 mg/mL of the bispecific GPRC5D/CD3 antibody, about 15 mM of acetate and/or a pharmaceutically acceptable acetate salt, about 8% (w/v) sucrose, about 18 μg/mL of EDTA, and about 0.04% polysorbate (PS) 20, wherein the stable aqueous pharmaceutical composition has a pH of about 5.2.
   36. The method of embodiment 34 or 35, wherein the bispecific GPRC5D/CD3 antibody comprises a VH1 having the amino acid sequence of SEQ ID NO:7, and a VL1 having the amino acid sequence of SEQ ID NO:8.
   37. The method of embodiment 34 or 35, wherein the bispecific GPRC5D/CD3 antibody comprising a HC1 having the amino acid sequence of SEQ ID NO:9, and a LC1 having the amino acid sequence of SEQ ID NO:10.
   38. The method of embodiment 34 or 35, wherein the bispecific GPRC5D/CD3 antibody comprises a VH2 having the amino acid sequence of SEQ ID NO:17, and a VL2 having the amino acid sequence of SEQ ID NO:18.
   39. The method of embodiment 34 or 35, wherein the bispecific GPRC5D/CD3 antibody comprises a HC2 having the amino acid sequence of SEQ ID NO:19, and a LC2 having the amino acid sequence of SEQ ID NO:20.
   40. The method of embodiment 34 or 35, wherein the bispecific GPRC5D/CD3 antibody is talquetamab.
   41. The method of any one of embodiments 34-40, wherein the aqueous pharmaceutical composition is stable.
   42. The method of embodiment 41, wherein stability is defined based on color of solution, pH, turbidity, percentage of purity, percentage of new peaks, percentage of main component, percentage of high molecular weight species (HWMS), percentage of low molecular weight species (LMWS), percentage of sum of acidic peaks, percentage of sum of basic peaks, protein concentration, percentage of T cell activation, percentage of PS 20 (w/v), or any combination thereof.
   43. A kit comprising the aqueous pharmaceutical composition of any one of embodiments 1a, 1b, or 2-31 and instructions for use.
   44. An article of manufacture comprising a container holding the aqueous pharmaceutical composition of any one of embodiments 1a, 1b, or 2-31.
   45. The article of manufacture of embodiment 44, wherein the container is a vial with a stopper pierceable by a syringe.
   46. The aqueous pharmaceutical composition of any one of embodiments 1a, 1b, or 2-31 for use in the treatment of cancer.
   47. The aqueous pharmaceutical composition of any one of embodiments 1a, 1b, or 2-31 for use in the preparation of a medicament for the treatment of cancer.
   48. Use of the aqueous pharmaceutical composition of any one of embodiments 1a, 1b, or 2-31 for treating a cancer in a subject in need thereof, the use comprising administering the aqueous pharmaceutical composition to the subject in need thereof.
   49. The use of embodiment 48, wherein the administration is subcutaneous.
   50. The method of embodiment 32 or 33, wherein the human subject is an adult with relapsed or refractory multiple myeloma and has received at least four prior therapies, including a proteasome inhibitor, an immunomodulatory agent and an anti-CD38 monoclonal antibody.
   51. The method of embodiment 32 or 33, wherein the human subject is an adult with relapsed and refractory multiple myeloma, and has received at least 3 prior therapies, including an immunomodulatory agent, a proteasome inhibitor, and an anti-CD38 antibody and has demonstrated disease progression or did not respond to the last therapy.
   52a. The method of embodiment 50 or 51 comprising administering the aqueous pharmaceutical composition on a dosing schedule according to the subject's body weight, wherein said dosing schedule comprises:
subcutaneously administering the aqueous pharmaceutical composition to the subject at step-up doses of 0.01 mg/kg, 0.06 mg/kg, 0.4 mg/kg, and 0.8 mg/kg on days 1, 3, 5, and 7, respectively, followed by subcutaneously administering the aqueous pharmaceutical composition to the subject at a treatment dose of 0.8 mg/kg once every two weeks,
wherein the aqueous pharmaceutical composition comprising 2 mg/mL of talquetamab is administered for the doses of 0.01 mg/kg and 0.06 mg/kg and the aqueous pharmaceutical composition comprising 40 mg/mL of talquetamab is administered for each dose of 0.4 mg/kg and 0.8 mg/kg.
   52b. The method of embodiment 50 or 51 comprising administering the aqueous pharmaceutical composition on a dosing schedule according to the subject's body weight, wherein said dosing schedule comprises:
subcutaneously administering the aqueous pharmaceutical composition to the subject at step-up doses of 0.01 mg/kg, 0.06 mg/kg, 0.3 mg/kg, and 0.8 mg/kg on days 1, 3, 5, and 7, respectively, followed by subcutaneously administering the aqueous pharmaceutical composition to the subject at a treatment dose of 0.8 mg/kg once every two weeks,
wherein the aqueous pharmaceutical composition comprising 2 mg/mL of talquetamab is administered for the doses of 0.01 mg/kg and 0.06 mg/kg and the aqueous pharmaceutical composition comprising 40 mg/mL of talquetamab is administered for each dose of 0.3 mg/kg and 0.8 mg/kg.
   53. The method of embodiment 50 or 51 comprising administering the aqueous pharmaceutical composition on a dosing schedule according to the subject's body weight, wherein said dosing schedule comprises:
subcutaneously administering the aqueous pharmaceutical composition to the subject at step-up doses of 0.01 mg/kg, 0.06 mg/kg and 0.4 mg/kg, on days 1, 3 and 5, respectively, followed by subcutaneously administering the aqueous pharmaceutical composition to the subject at a treatment dose of 0.4 mg/kg once per week,
wherein the aqueous pharmaceutical composition comprising 2 mg/mL of talquetamab is administered for the doses of 0.01 mg/kg and 0.06 mg/kg and the aqueous pharmaceutical composition comprising 40 mg/mL of talquetamab is administered for each dose of 0.4 mg/kg.
   54. The method of embodiment 52a, 52b, or 53, wherein said aqueous pharmaceutical composition comprising 2 mg/mL of talquetamab has the following composition per 1.5 mL: 3 mg of talquetamab, edetate disodium (0.027 mg), glacial acetic acid (0.36 mg), polysorbate 20 (0.6 mg), sodium acetate (1.39 mg), sucrose (120 mg), and Water for Injection, USP.
   55. The method of any of embodiments 52a, 52b, 53, or 54, wherein said aqueous pharmaceutical composition comprising 40 mg/mL of talquetamab has the following composition per 1 mL: 40 mg of talquetamab, edetate disodium (0.018 mg), glacial acetic acid (0.24 mg), polysorbate 20 (0.4 mg), sodium acetate (0.90 mg), sucrose (80 mg), and Water for Injection, USP.
   56. The method of any of embodiments 50, 51, 52a, 52b, or 53-55, further comprising administering pretreatment medications 1-3 hours before each step-up dose, wherein said pretreatment medications comprise: Corticosteroid (oral or intravenous dexamethasone, 16 mg or equivalent); Antihistamines (oral or intravenous diphenhydramine, 50 mg or equivalent); and Antipyretics (oral or intravenous acetaminophen, 650 mg to 1000 mg or equivalent).
   57. The method of any of embodiments 50, 51, 52a, 52b, or 53-55, wherein the method is effective in eliciting a partial response, a very good partial response, a complete response, or a stringent complete response in the subject according to IMWG criteria.
   58. The method of any of embodiments 50, 51, 52a, 52b, or 53-55, wherein the method results in an overall response rate (ORR) of at least 70% in a population of the human subjects, according to IMWG criteria.
   59. An aqueous pharmaceutical composition of any one of embodiments 1a, 1b, or 2-31 comprising talquetamab,
wherein subcutaneous administration of the composition to a population of patients diagnosed with released or refractory multiple myeloma elicits an overall response rate (ORR) of at least 70% in said population (according to IMWG criteria) following administration based on each patient's body weight according to a dosing schedule comprising subcutaneous administration of the composition at doses of 0.01 mg/kg, 0.06 mg/kg and 0.4 mg/kg, on days 1, 3 and 5, respectively, followed by subcutaneous administration of the composition to the subject at a dose of 0.4 mg/kg once per week,
wherein the composition comprising 2 mg/mL of talquetamab is administered for the doses of 0.01 mg/kg and 0.06 mg/kg and the aqueous pharmaceutical composition comprising 40 mg/mL of talquetamab is administered for each dose of 0.4 mg/kg,
wherein each patient in the population has received at least three or four prior therapies, including a proteasome inhibitor, an immunomodulatory agent and an anti-CD38 monoclonal antibody.
   60. An aqueous pharmaceutical composition of any one of embodiments 1a, 1b, or 2-31 comprising talquetamab,
wherein subcutaneous administration of the composition to a population of patients diagnosed with released or refractory multiple myeloma elicits an overall response rate (ORR) of at least 70% in said population (according to IMWG criteria) following administration based on each patient's body weight according to a dosing schedule comprising subcutaneous administration of the composition at doses of 0.01 mg/kg, 0.06 mg/kg, 0.3 mg/kg, and 0.8 mg/kg on days 1, 3, 5, and 7, respectively, followed by subcutaneous administration of the composition to the subject at a dose of 0.8 mg/kg every two weeks,
wherein the composition comprising 2 mg/mL of talquetamab is administered for the doses of 0.01 mg/kg and 0.06 mg/kg and the aqueous pharmaceutical composition comprising 40 mg/mL of talquetamab is administered for each dose of 0.3 mg/kg and 0.8 mg/kg,
wherein each patient in the population has received at least three or four prior therapies, including a proteasome inhibitor, an immunomodulatory agent and an anti-CD38 monoclonal antibody.
   61. An aqueous pharmaceutical composition of any one of embodiments 1a, 1b, or 2-31 comprising talquetamab,
wherein subcutaneous administration of the composition to a population of patients diagnosed with released or refractory multiple myeloma elicits an overall response rate (ORR) of at least 70% in said population (according to IMWG criteria) following administration based on each patient's body weight according to a dosing schedule comprising subcutaneous administration of the composition at doses of 0.01 mg/kg, 0.06 mg/kg, 0.4 mg/kg, and 0.8 mg/kg on days 1, 3, 5, and 7, respectively, followed by subcutaneous administration of the composition to the subject at a dose of 0.8 mg/kg every two weeks,
wherein the composition comprising 2 mg/mL of talquetamab is administered for the doses of 0.01 mg/kg and 0.06 mg/kg and the aqueous pharmaceutical composition comprising 40 mg/mL of talquetamab is administered for each dose of 0.4 mg/kg and 0.8 mg/kg,
wherein each patient in the population has received at least three or four prior therapies, including a proteasome inhibitor, an immunomodulatory agent and an anti-CD38 monoclonal antibody.

### EXAMPLES

The following examples are provided to further describe some of the embodiments disclosed herein. The examples are intended to illustrate, not to limit, the disclosed embodiments.

### Description of Analytical Tests Used Herein

### Analytical Tests—General Characterization

### Color of Solution

Color of solution is monitored for drug product to assess appearance and ensure it is consistent with previous batches at release and over the shelf life. Color of solution may be an indicator of product stability. To determine Color of solution, test samples are visually compared to a defined set of reference solutions.

A defined volume of liquid content is transferred into a pre-scored ampoule of same dimensions as the reference solutions. Then the content of the ampoule is visually compared to European Pharmacopoeia color reference solutions. The degree of color is determined in diffuse daylight, viewed against a white background.

### Color of Solution Material and Methods

Materials and methods are as described in European Pharmacopoeia 2.2.2, Degree of Coloration of Liquids European Pharmacopoeia (Ph. Eur.) 10th Edition monograph number 20202, July 2019. Briefly, test articles are compared against B (Brown), BY (Brownish-Yellow), and Y (Yellow) Color Reference Solution Sets. Stability of the aqueous pharmaceutical composition as determined by color of solution is defined as provided for herein.

pH

pH Materials and Methods—A daily calibrated electronic pH meter with standardized pH electrode is used to measure the pH of test articles. All calibration solutions, reference buffers, and test articles are equilibrated to, and maintained at, 25° C. prior to and during testing. Stability of the aqueous pharmaceutical composition as determined by pH is defined as provided for herein.

### Turbidity

Turbidity Materials and Methods—The materials and methods are based on European Pharmacopoeia 2.2.1, Clarity and Degree of Opalescence of Liquids. Stability of the aqueous pharmaceutical composition as determined by turbidity is defined as provided for herein.

### Analytical Tests—Particulate Matter

Particulate Matter (Sub-visible) Materials and Methods—All materials and methods are compliant with United States Pharmacopeia <788> Particulate Matter. A Compendial compliant Liquid Particle Counter instrument equipped with a compendial volume sampler set-up is used. Test articles are equilibrated to room temperature for at least 60 minutes, but no longer than 10 hours, prior to testing. Test article vials are pooled in manner compliant with United States Pharmacopeia <788> Particulate Matter. As instructed by United States Pharmacopeia <788> Particulate Matter, four portions of pooled test article, each of appropriate volume, are removed and the number of particles equal to or greater than 10 μm and 25 μm are counted per portion. Results obtained for the first portion are disregarded and the remaining three results are used to calculate the mean number of particles for the preparation examined.

Particle Analysis (sub-vis) compendia compliant results—Testing results are to comply with United States Pharmacopoeia <788> Particulate Matter, European Pharmacopoeia 2.9.19, and Japanese Pharmacopoeia XVII/6.07 Particulate Contamination: Sub-visible particles. As such, the average number of particles present in the units tested should not exceed 6000 particles per container for particles size equal to 10 μm or greater and should not exceed 600 particles per container for particles size equal to 25 μm or greater.

### Analytical Tests—Purity

Capillary Electrophoresis Sodium Dodecyl Sulfate (cSDS)-Reduced

cSDS Reduced Materials and Methods—Analysis employs a commercial capillary electrophoresis system with a bare fused silica capillary, 50 μm i.d.×30.2 cm length in a temperature-controlled cartridge; the capillary is equipped with a detection window transparent to ultraviolet light. The capillary is rinsed electrokinetically before each injection. The capillary is loaded with a sieving matrix consisting of an entangled polymer solution before each sample analysis. The method utilizes an SDS-MW gel migration buffer and certified protein molecular weight standards spanning a range of approximately 10 to 148 kDa. The instrument's ultraviolet absorption spectrophotometer detector is set at a wavelength of 220 nm and the capillary temperature is set to 25° C. For reducing sample treatment conditions, the test article (in duplicate) is mixed with SDS and 2-mercaptoethanol and then heated for a defined time and temperature to fully denature and reduce the protein. The reduced sample is injected electro-kinetically by applying a voltage of 5 kV across the capillary for approximately 20 seconds, and then analyzed by application of a greater electric field for approximately 35 minutes. Detection is accomplished by absorbance in the far ultraviolet region of the spectrum, 220 nm. Percent of total signal data is collected for the light chain, heavy chain, and a glycosylated heavy chain (AG HC). Stability of the aqueous pharmaceutical composition as determined by cSDS is defined as provided for herein.

Capillary Electrophoresis Sodium Dodecyl Sulfate (cSDS)-Non-Reduced

cSDS Non-reduced Materials and Methods—Analysis employs a commercial capillary electrophoresis system with a bare fused silica capillary, 50 μm i.d.×30.2 cm length in a temperature-controlled cartridge; the capillary is equipped with a detection window transparent to ultraviolet light. The capillary is rinsed electrokinetically before each injection. The capillary is loaded with a sieving matrix consisting of an entangled polymer solution before each sample analysis. The method utilizes an SDS-MW gel migration buffer, certified protein molecular weight standards spanning a range of approximately 10 to 148 kDa, and a validated talquetamab reference material sample. The instrument's ultraviolet absorption spectrophotometer detector is set at a wavelength of 220 nm and the capillary temperature is set to 25° C. For non-reduced sample treatment conditions, the test article (in duplicate) is mixed with SDS and the alkylating reagent (N-Ethylmaleimide, to prevent disulfide bond shuffling or reformation). It is then heated for a defined time and temperature to fully denature the protein and minimize formation of fragments and artifact bands. The non-reduced sample is injected electrokinetically by applying a voltage of 5 kV across the capillary for approximately 20 seconds, and then analyzed by application of a greater electric field for approximately 35 minutes. Detection is accomplished by absorbance in the far ultraviolet region of the spectrum, 220 nm. Percent of total signal data is collected. The data is also analyzed for the presence of new peaks versus talquetamab reference material. Percent purity is defined as percent heavy chain+percent light chain. Stability of the aqueous pharmaceutical composition as determined by cSDS is defined as provided for herein.

### Size Exclusion High Performance Liquid Chromatography (SE-HPLC)

SE-HPLC Materials and Methods—Reference Material and test articles are diluted to a target protein concentration. A 20 μl volume of analyte is injected onto a 7.8 mm×30 cm size exclusion column with 5 μm particle size silica base, with a fractionation range of 10 to 500 kDa. Aqueous phosphate buffer is used as the mobile phase at a flow rate of 0.7 mL/minute and the absorbance of the eluate is monitored continuously at 280 nm. Monomer (main component or main peak), aggregates (high molecular weight species, or HMWS), and fragments (low molecular weight species, or LMWS) are separated on the column and elute at different retention times. The amounts of these species are measured by monitoring peak absorbance at 280 nm. Stability of the aqueous pharmaceutical composition as determined by SE-HPLC is defined as provided for herein. Stability of the aqueous pharmaceutical composition as determined by the Main Component (MC) of SE-HPLC is defined as provided for herein. Stability of the aqueous pharmaceutical composition as determined by the HMWS of SE-HPLC is defined as provided for herein. Stability of the aqueous pharmaceutical composition as determined by the LMWS of SE-HPLC is defined as provided for herein.

### Ion Exchange Chromatography (IEX)

### IEX Materials and Methods—

The analytical procedure was performed using a HPLC system with diode array or UV detector, column heating capability, and temperature controlled HPLC auto-injector. The stationary phase was a MAbPac SCX-10, 4×250 mm, 10 m particle size, cation exchange column maintained at 35° C.+/−2° C. Mobile Phase A was ThermoFisher CX-1 pH Gradient Buffer A, pH 5.6 diluted 1:10 in DI water. Mobile Phase B was ThermoFisher CX-1 pH Gradient Buffer B, pH 10.2 diluted 1:10 in DI water. Reference Material and test articles are diluted to a target protein concentration. 10-100 μl aliquots were transferred to sample vials and place in the temperature control HPLC auto-injector set at 5° C.+/−2° C. The run time per sample was 16 minutes at a flow rate of 0.75 mL/minute. A gradient, mediated by mobile phase A and B, was applied which incrementally increased the mobile phase pH during the run. As the pH increases, isoforms becomes neutral and elutes from the column. The eluate is monitored continuously at 280 nm and the relative amount of each isoform is measured by comparing peak area count. The largest peak observed is designated the main peak. All peaks/groups eluted prior to main peak as designated acidic species and all peaks/groups eluted after the main peak as designated basic species. Stability of the aqueous pharmaceutical composition as determined by IEX is defined as provided for herein. Stability of the aqueous pharmaceutical composition as determined by the Main Peak (MP) of IEX is defined as provided for herein. Stability of the aqueous pharmaceutical composition as determined by the sum of acidic peaks of IEX is defined as provided for herein. Stability of the aqueous pharmaceutical composition as determined by the sum of basic peaks of IEX is defined as provided for herein.

### Analytical Tests—Quantity

### Protein Concentration by A280

Protein concentration of the drug product is determined by quantification of the absorbance at 280 nm (A280).

### Protein Concentration by A280 Materials and Methods

Measurement of protein concentration is performed using a qualified and calibrated double beam UV-Vis spectrophotometer. 40 mg/ml talquetamab test articles are diluted 1:80 using 0.9% (w/v) NaCl. 2 mg/mL talquetamab test articles are diluted 1:6.7 using 0.9% (w/v) NaCl. Samples are measured using quartz semi-micro cuvettes (1.4 ml) with a 1 cm path length and black or frosted sides. The Spectrophotometer is set to a Wavelength of 280 nm, a slit width of 1 nm, and a response of one (1) second. 0.9% (w/v) NaCl is used as the Blank control. Protein concentration (mg/mL) is calculated by dividing the product of the Test article absorbance and dilution factor by the product of the antibody's Absorptivity Constant and instrument's path length (for example, but not limited to talquetamab's Absorptivity Constant of 1.49 (mg/mL)⁻¹ cm⁻¹ and instrument's path length of 1 cm). Stability of the aqueous pharmaceutical composition as determined by A280 protein concentration is defined as provided for herein. Stability of the 2 mg/mL aqueous pharmaceutical composition as determined by A280 protein concentration is defined as provided for herein. Stability of the 40 mg/mL aqueous pharmaceutical composition as determined by A280 protein concentration is defined as provided for herein.

### Analytical Tests—Potency

### Potency (GPRC5D/CD3 T-Cell Activation)

The in vitro T-cell activation the GPRC5D/CD3 bispecific antibodies provided for herein is demonstrated using a Nuclear factor of activated T cells-Response Element (NFAT-RE)-mediated luminescence assay.

This reporter assay uses luminescence induced by activation of the NFAT (nuclear factor of activated T cells) pathway in CD3-expressing engineered effector cells as a read-out for target cell/effector cell co-engagement and is a surrogate measure of target cell killing. MM.1R B lymphoblast cells, which expresses GPRC5D on its cell surface, are used as the target cells. The engagement of both the anti-GPRC5D Fab region with the GPRC5D expressing target cells and the anti-CD3 Fab region with the genetically engineered CD3+ T-cells are required for T-cell activation and subsequent NFAT-RE-mediated luminescence.

GPRC5D/CD3 T-cell Activation Materials and Methods. Qualified talquetamab is used as Reference Material and Controls. Solutions of Jurkat TCR/CD3 effector cells at 1.3×10⁶ viable cells/mL in culture media (4% Heat-Inactivated Fetal Bovine Serum in RPMI) and a solution of MM.1R B lymphoblast target cells at 1.75×10⁵ viable cells/mL in cell culture media are prepared. Equal volumes of effector cells and target cells are aliquoted into individual wells on a 96-well cell culture plate. The plate is then incubated at 37° C. (±2° C.) with 5% (±2%) CO₂ for 16-24 hours. After incubation, Bio-Glo™ Luciferase substrate is added to each well and agitated moderately on a plate shaker for at least 5 minutes. Within 30 minutes of the addition of the substrate, luminescence (Relative Light Unit (RLU) values) is measured using a microtiter luminescence plate reader. Data is reported as percent bioactivity relative to Reference Material. Stability of the aqueous pharmaceutical composition as determined by GPRC5D/CD3 T-Cell activation activity is defined as provided for herein.

### Analytical Tests—Surfactant

### Polysorbate-20 Quantification

Polysorbate 20 is quantitatively determined by mixed-mode ion-exchange/hydrophobic HPLC.

PS 20 Materials and Methods. Analysis conducted with a gradient HPLC equipped with a 2.1×20 mm on-line column containing a 30 μm water-wettable, mixed-mode polymeric spherical sorbent particles, an ELSD, and a temperature-controlled column compartment at 30° C. The flow rate is set to 1 mL/minute and the ELSD nebulizer temperature and evaporator temperature are both set to 50° C. Mobile Phase A is 2% v/v Formic acid in water and Mobile Phase B is 2% v/v Formic acid in Isopropyl alcohol. Neat Polysorbate 20 is used to create calibration and check standards. Test article samples are injected neat. Stability of the aqueous pharmaceutical composition as determined by polysorbate 20 quantification is defined as provided for herein.

### Analytical Tests—Routine Characterization

### Peptide Map

The purpose of this test is to measure the levels of post-translational modifications, such as oxidation, deamidation, and isomerization, that may be present in the antibody structure. Test articles are enzymatically digested to yield peptide segments. These peptides are then evaluated by Ultra High-Performance Liquid Chromatography Mass Spectroscopy (UPLC-MS). Each analyzed peptide sequence is identified relative to its known location within the overall antibody structure. Post-translational modifications are determined by comparing the measured mass of the identified peptide sequence with its expected mass.

Peptide Mapping materials and methods. Samples are denatured with 6 M Guanidine, 50 mM Tris pH 8.0, 5 mM EDTA and filtered using 30 kDa centrifugal filter device (flow through discarded). The denatured samples are reduced with 1 M Dithiothreitol (DTT), followed by alkylation with 1 M sodium Iodoacetate, and further treated with DTT to quench the reaction. The reaction mixture is exchanged into digestion buffer (50 mM Tris pH 7.0, with 1 mM CaCl₂) via Sephadex G-25 columns with separate columns used for blanks, reference material, and test articles. An aliquot of 1 mg/mL Trypsin stock solution is added to the sample in digestion buffer yielding a 20 μL/mL trypsin concentration. The solution is incubated at 37° C. for 2 hours±30 minutes. The trypsinized solution is allowed to cool to room temperature and the enzyme is inactivated with Trifluoroacetic acid. The treated samples are evaluated by Ultra High-Performance Liquid Chromatography Mass Spectroscopy (UPLC-MS) equipped with a Waters Acquity BEH (Ethylene Bridged Hybrid) C18, 2.1×100 mm, 1.7 μm, 130 Å column and an attached auto sampler. Mobile phase A is 0.1% Formic Acid in water Mobile phase B is, 0.1% FA in acetonitrile (mobile phase B). The autosampler is set to 2-8° C., the column is set to 40° C. and the flow rate is set to 500 μL/minute. Eluted peptides are subjected to electrospray ionization and detected using a calibrated on-line mass spectrometry.

### Example 1: Early Formulation Development

### High-Throughput (HTP) Multi-Factorial Screening Studies

High-throughput (HTP) multi-factorial screening studies were conducted to select combinations of formulation buffer species and concentration, excipient species, and pH. This is achieved by generating multiple test formulations consisting of various combinations of buffers species and concentrations, excipients, and pH values. The target antibody concentration used for all test formulations was 2 mg/mL. Sucrose was evaluated at 8.5% (w/v) and sorbitol at 5.0% (w/v).

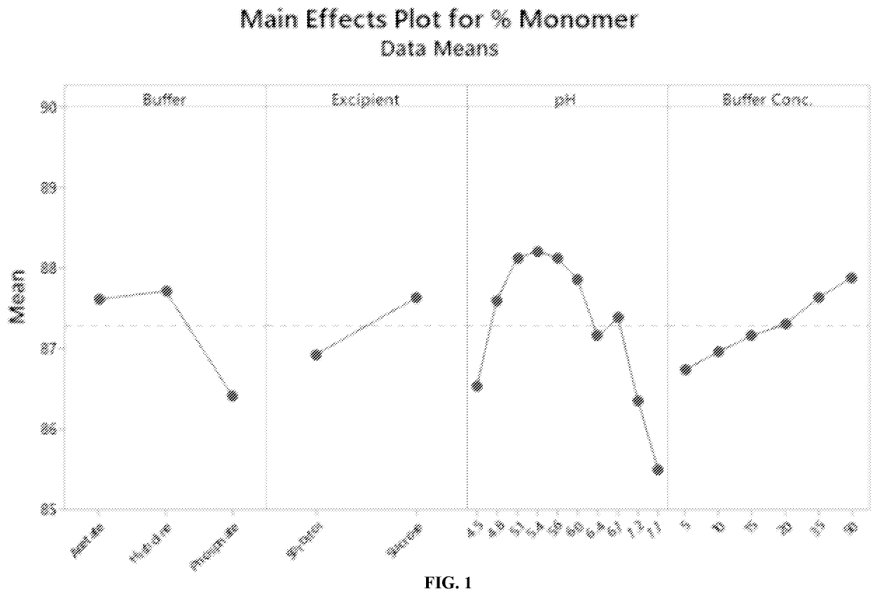

In this study, approximately 200 μL of each test formulation was aliquoted in to 96 well plates, sealed to prevent evaporation, and held under stressed (40° C.) stability conditions for three weeks. The test formulations were then analyzed by SEC. The composition of the test formulations and their corresponding SEC results are list per buffer in Tables 7, 8, and 9 below. Main effects plots calculated from all test formulation data were generated using Minitab 19.0 statistical software (FIGS. 1, 2 and 3).

TABLE 7
The composition of the acetate formulations and
corresponding SEC results

| | | | Buffer | % | % | % |
|---|---|---|---|---|---|---|
| Buffer | Excipient | pH | Conc. | Monomer | Aggregate | Fragment |
| Acetate | Sorbitol | 4.5 | 5 | 87.54 | 3.91 | 8.54 |
| | | | 10 | 87.06 | 3.95 | 8.99 |
| | | | 15 | 86.65 | 3.97 | 9.38 |
| | | | 20 | 86.33 | 3.96 | 9.71 |
| | | | 35 | 85.86 | 4.05 | 10.09 |
| | | | 50 | 85.87 | 4.13 | 10.01 |
| | | 4.8 | 5 | 88.11 | 4.7 | 7.18 |
| | | | 10 | 87.85 | 4.73 | 7.42 |
| | | | 15 | 87.82 | 4.67 | 7.5 |
| | | | 20 | 87.45 | 4.65 | 7.89 |
| | | | 35 | 87.37 | 4.66 | 7.97 |
| | | | 50 | 87.2 | 4.68 | 8.12 |
| | | 5.1 | 5 | 88.08 | 5.43 | 6.5 |
| | | | 10 | 88.03 | 5.45 | 6.51 |
| | | | 15 | 88.02 | 5.37 | 6.6 |
| | | | 20 | 87.92 | 5.38 | 6.7 |
| | | | 35 | 87.95 | 5.21 | 6.84 |
| | | | 50 | 88.1 | 5.17 | 6.73 |
| | | 5.4 | 5 | 87.87 | 6.33 | 5.8 |
| | | | 10 | 87.85 | 6.51 | 5.63 |
| | | | 15 | 87.94 | 6.49 | 5.56 |
| | | | 20 | 87.81 | 6.43 | 5.76 |
| | | | 35 | 88.48 | 6.12 | 5.41 |
| | | | 50 | 88.54 | 5.94 | 5.51 |
| | Sucrose | 4.5 | 5 | 87.65 | 3.87 | 8.48 |
| | | | 10 | 86.72 | 3.9 | 9.39 |
| | | | 15 | 86.59 | 4.02 | 9.4 |
| | | | 20 | 86.57 | 3.98 | 9.45 |
| | | | 35 | 85.82 | 4.12 | 10.07 |
| | | | 50 | 85.64 | 4.33 | 10.03 |
| | | 4.8 | 5 | 87.98 | 4.55 | 7.48 |
| | | | 10 | 87.78 | 4.57 | 7.65 |
| | | | 15 | 87.69 | 4.51 | 7.8 |
| | | | 20 | 87.69 | 4.57 | 7.74 |
| | | | 35 | 87.09 | 4.48 | 8.43 |
| | | | 50 | 87.1 | 4.6 | 8.3 |
| | | 5.1 | 5 | 88.3 | 5.05 | 6.65 |
| | | | 10 | 88.3 | 5.16 | 6.54 |
| | | | 15 | 88.15 | 5.08 | 6.76 |
| | | | 20 | 88.09 | 5.08 | 6.82 |
| | | | 35 | 88.11 | 4.86 | 7.04 |
| | | | 50 | 88.36 | 4.77 | 6.87 |
| | | 5.4 | 5 | 88.21 | 5.74 | 6.05 |
| | | | 10 | 88.16 | 5.91 | 5.93 |
| | | | 15 | 88.28 | 5.82 | 5.9 |
| | | | 20 | 88.2 | 5.71 | 6.09 |
| | | | 35 | 88.47 | 5.38 | 6.15 |
| | | | 50 | 88.72 | 5.21 | 6.08 |

TABLE 8
The composition of the histidine formulations and
corresponding SEC results

| | | | Buffer | % | % | % |
|---|---|---|---|---|---|---|
| Buffer | Excipient | pH | Conc. | Monomer | Aggregate | Fragment |
| Histidine | Sorbitol | 5.6 | 5 | 88.21 | 6.09 | 5.7 |
| | | | 10 | 87.84 | 6.13 | 6.03 |
| | | | 15 | 87.84 | 6.03 | 6.14 |
| | | | 20 | 88.14 | 5.88 | 5.98 |
| | | | 35 | 88.24 | 5.61 | 6.16 |
| | | | 50 | 88.2 | 5.44 | 6.36 |
| | | 6 | 5 | 87.38 | 7.51 | 5.11 |
| | | | 10 | 87.02 | 7.79 | 5.19 |
| | | | 15 | 87.26 | 7.84 | 4.9 |
| | | | 20 | 87.17 | 7.73 | 5.09 |
| | | | 35 | 87.98 | 7.02 | 4.99 |
| | | | 50 | 88.47 | 6.47 | 5.06 |
| | | 6.4 | 5 | 86.72 | 8.71 | 4.56 |
| | | | 10 | 86.39 | 9.25 | 4.36 |
| | | | 15 | 86.18 | 9.32 | 4.5 |
| | | | 20 | 86.26 | 9.28 | 4.46 |
| | | | 35 | 86.97 | 8.56 | 4.47 |
| | | | 50 | 87.89 | 7.74 | 4.37 |
| | Sucrose | 5.6 | 5 | 88.41 | 5.61 | 5.98 |
| | | | 10 | 88.13 | 5.63 | 6.23 |
| | | | 15 | 88.24 | 5.47 | 6.3 |
| | | | 20 | 88.12 | 5.49 | 6.39 |
| | | | 35 | 88.1 | 5.18 | 6.71 |
| | | | 50 | 88.02 | 5.12 | 6.87 |
| | | 6 | 5 | 88.18 | 6.54 | 5.28 |
| | | | 10 | 87.78 | 6.83 | 5.39 |
| | | | 15 | 87.77 | 6.85 | 5.38 |
| | | | 20 | 88.11 | 6.7 | 5.2 |
| | | | 35 | 88.36 | 6.23 | 5.41 |
| | | | 50 | 88.82 | 5.87 | 5.3 |
| | | 6.4 | 5 | 87.67 | 7.49 | 4.84 |
| | | | 10 | 87.24 | 7.94 | 4.83 |
| | | | 15 | 87.21 | 8.03 | 4.76 |
| | | | 20 | 87.32 | 7.94 | 4.75 |
| | | | 35 | 87.71 | 7.52 | 4.77 |
| | | | 50 | 88.3 | 6.93 | 4.77 |

TABLE 9
The composition of the phosphate formulations and
corresponding SEC results

| | | | Buffer | % | % | % |
|---|---|---|---|---|---|---|
| Buffer | Excipient | pH | Conc. | Monomer | Aggregate | Fragment |
| Phosphate | Sorbitol | 6.7 | 5 | 84.47 | 11.51 | 4.02 |
| | | | 10 | 85.86 | 10.12 | 4.02 |
| | | | 15 | 86.71 | 9.29 | 3.99 |
| | | | 20 | 87.04 | 8.65 | 4.32 |
| | | | 35 | 88.17 | 7.72 | 4.11 |
| | | | 50 | 88.45 | 7.52 | 4.03 |
| | | 7.2 | 5 | 82.47 | 13.47 | 4.05 |
| | | | 10 | 84.35 | 11.33 | 4.32 |
| | | | 15 | 85.32 | 10.39 | 4.29 |
| | | | 20 | 86.06 | 9.7 | 4.24 |
| | | | 35 | 87.01 | 8.55 | 4.44 |
| | | | 50 | 87.49 | 8.18 | 4.34 |
| | | 7.7 | 5 | 80.96 | 15.26 | 3.79 |
| | | | 10 | 83.46 | 12.51 | 4.03 |
| | | | 15 | 84.18 | 11.76 | 4.06 |
| | | | 20 | 84.86 | 10.76 | 4.39 |
| | | | 35 | 86.35 | 9.43 | 4.22 |
| | | | 50 | 86.63 | 9.13 | 4.23 |
| | Sucrose | 6.7 | 5 | 86.44 | 9.27 | 4.29 |
| | | | 10 | 87.15 | 8.23 | 4.62 |
| | | | 15 | 87.86 | 7.65 | 4.49 |
| | | | 20 | 88.43 | 7.16 | 4.41 |
| | | | 35 | 88.86 | 6.61 | 4.52 |
| | | | 50 | 89.14 | 6.26 | 4.59 |
| | | 7.2 | 5 | 85.22 | 10.29 | 4.49 |
| | | | 10 | 86.55 | 8.81 | 4.64 |
| | | | 15 | 87.12 | 8.2 | 4.68 |
| | | | 20 | 87.58 | 7.76 | 4.66 |
| | | | 35 | 88.28 | 7.01 | 4.71 |
| | | | 50 | 88.7 | 6.67 | 4.63 |
| | | 7.7 | 5 | 84.88 | 10.51 | 4.6 |
| | | | 10 | 85.78 | 9.35 | 4.87 |
| | | | 15 | 86.33 | 8.93 | 4.74 |
| | | | 20 | 86.94 | 8.37 | 4.7 |
| | | | 35 | 87.47 | 7.76 | 4.77 |
| | | | 50 | 88.08 | 7.3 | 4.62 |

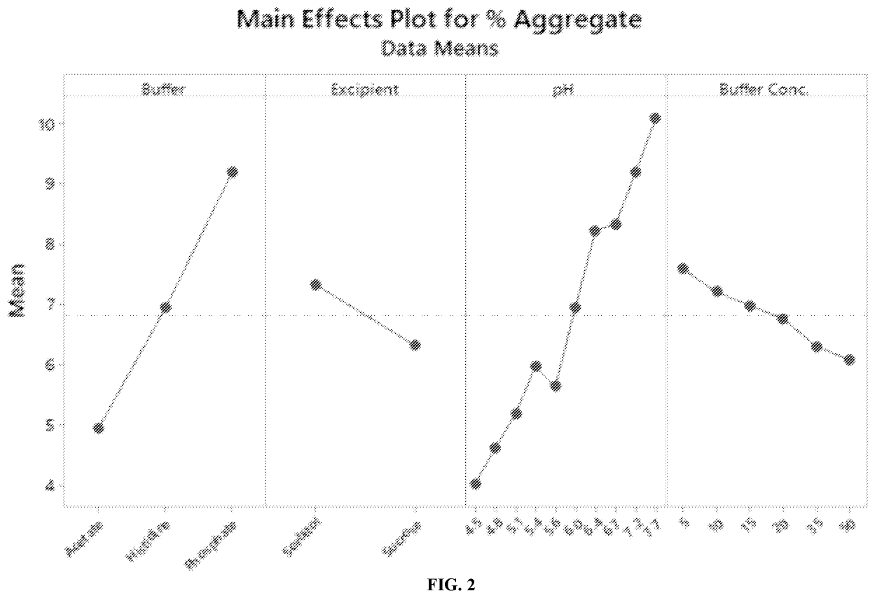

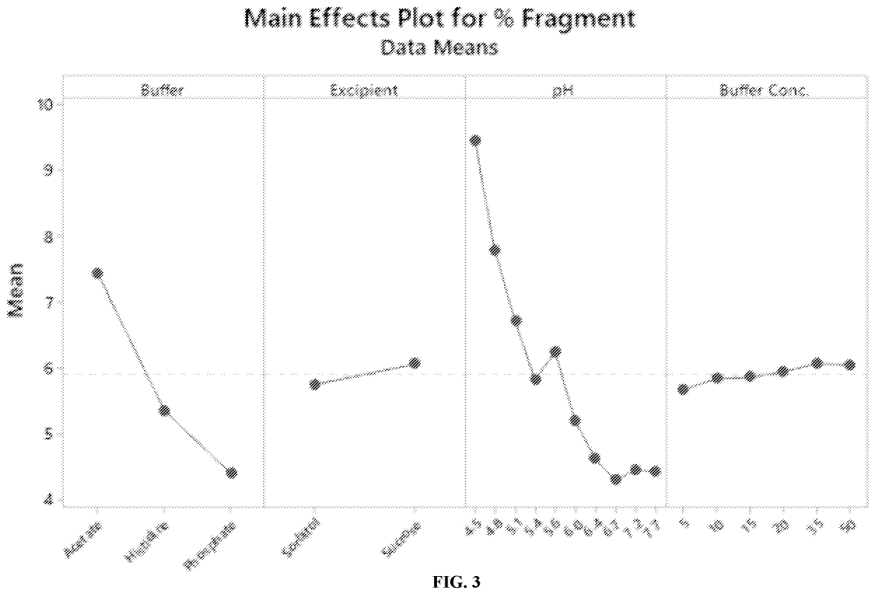

As seen in FIG. 1, pH appears to have a strong, non-linear, biphasic effect on the % monomer response with a pH value of approximately 5.4 at the apex of the curve. As reflected in FIGS. 2 and 3, respectively, test formulations above 5.4 primarily lose monomer to aggregation while test formulations below pH 5.4 appear to lose monomer to fragmentation. The monomer values at pH 5.1 and 5.6 are slightly lower than the monomer value at 5.4. However, the loss of monomer above 5.6 and below pH 5.1 appears to occur in a steep, non-linear manner inferring a finite pH range in which the formulation will remain stable. Based on the pH profile verse buffer species, histidine and phosphate were ruled out as buffers.

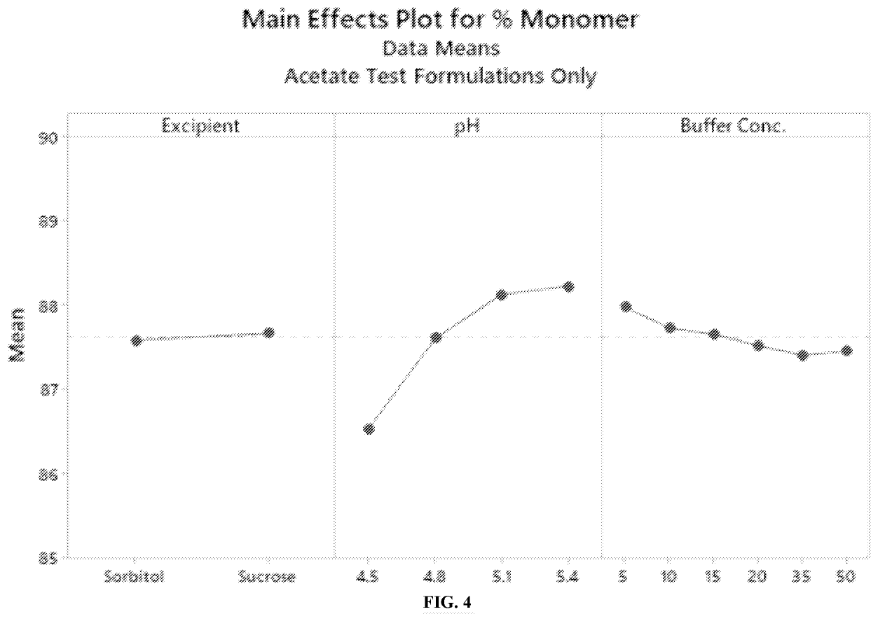

To further evaluate the acetate buffer, a second set of main effect plots were generated using data from the acetate test formulations only (FIGS. 4, 5, 6).

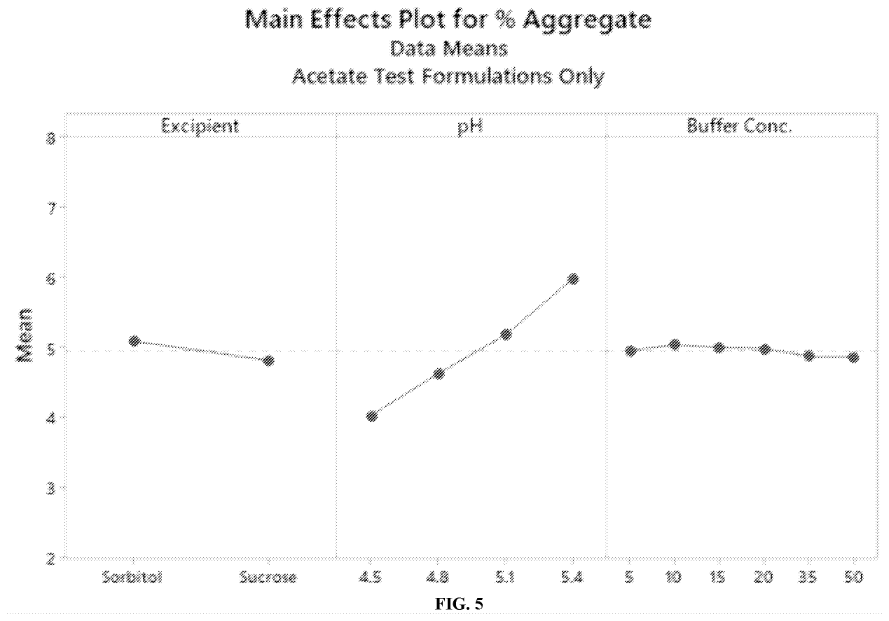

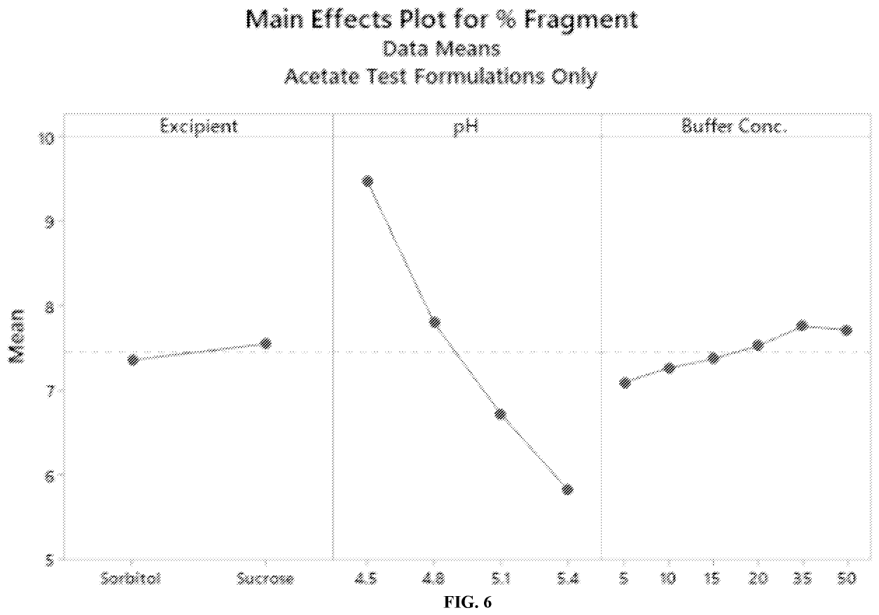

FIGS. 5 and 6 again show a strong linear effect of pH on aggregation and fragmentation respectively while FIG. 4 again shows the nonlinear effect of pH on monomer. However, the curve appears to begin to plateau at pH 5.1.

FIGS. 4 and 6 show decreased acetate buffer concentration correlating with increased stability while FIG. 5 shows acetate buffer concentration having no meaningful effect of aggregation. FIGS. 4 and 5 show sucrose exhibiting a slight increase in stability verses sorbitol while FIG. 6 shows sorbitol exhibiting an equally slight increase in stability verses sucrose.

Taken together, these early screening studies identified an optimal formulation consisting of an acetate buffer at low concentration and a pH between 5.1 and 5.4. Based on practical considerations, sucrose was selected as the excipient.

### Oxidative Stress Screening Study

A preliminary oxidative stress screening study was performed to evaluate the sensitivity (or lack thereof) of the DP to oxidation. Oxidative stress was mediated by the addition of trace metals to DP test formulations, and the test formulations were stored under stressed (40° C.) stability conditions for one month. As a mitigation strategy, EDTA was also added to test formulations to evaluate its ability to mitigate oxidation.

The study contained three experimental formulations: 1) No Metals, 2) With Metals, and 3) With Metals and EDTA. The No Metals test formulation consisted of 1 mg/mL DP in the 10 mM acetate, 8% (w/v) sucrose, 0.04% (w/v) PS20, pH 5.2. The With Metals test formulation consisted of the No Metals test formulation spiked with trace (low ppb) metals. The species of metals selected for this study were based on the composition of metal alloy components potentially present in manufacturing processes. The concentrations of the trace metals used in this study were 2× to 20× values of trace metals historically reported in literature during DP manufacturing processes. The With Metals and EDTA test formulation consistent of the With Metals test formulation supplemented with 20 μg/mL EDTA.

The test formulations were aliquoted into 8R vials at a fill volume of 3.3 mL. All vials were stoppered, capped, and crimp sealed. The vials were stored under stress (40° C.) stability conditions for one month. The samples then were pulled and assayed.

After one month under stress (40° C.) stability conditions, the No Metals test formulation showed an increase in HMWS as measured by SE-HPLC and a decrease in Purity as measured by both non-reduced and reduced cSDS (Table 10). These results are consistent with a monoclonal antibody stored under stress (40° C.) stability conditions for one month. The With Metals test formulation showed a dramatic increase HMWS and decrease in Purity as compared to the No Metals at the same time point. This data infers that, under stress (40° C.) stability conditions for one month, the species and concentration of trace metals used in this study contributed to increased degradation vs. similar test formulations without metals. However, the With Metals and EDTA test formulations showed assay values consistent with the No Metals. These data infer the addition of EDTA in the test formulation mitigated the apparent increased degrative effects seen of With Metals test formulations trace metals under stress (40° C.) stability conditions for one month.

TABLE 10
Impact of Metal Ions on DP Stability for 1 Month 40° C.

| | | Test Formulations | | | | With Metals | With Metals |
|---|---|---|---|---|---|---|---|
| | | No | No | | | and | and |
| | | Metals | Metals | With Metals | With Metals | EDTA | EDTA |
| Assay | Attribute | T = 0 | 1 m | T = 0 | 1 m | T = 0 | 1 m |
| SE-HPLC | HMWS (%) | 0.4 | 1.6 | 0.4 | 14.6 | 0.4 | 0.7 |
| | Main | 99.6 | 98.3 | 99.6 | 85.2 | 99.6 | 99.2 |
| | Component | | | | | | |
| | (%) | | | | | | |
| | LMWS | <0.1 | <0.1 | <0.1 | 0.2 | <0.1 | <0.1 |
| | (%) | | | | | | |
| cSDS | Purity | 98.1 | 94.6 | 98.2 | 73.5 | 98.2 | 95.7 |
| (non-reduced) | (%) | | | | | | |
| cSDS | Purity | 98.9 | 94.1 | 99 | 79.4 | 98.9 | 95.2 |
| (reduced) | (%) | | | | | | |

Based on the results of this preliminary oxidative stress screening study, EDTA was added to the DP formulation.

### Example 2: Polysorbate Concentration Range Shaking

A shaking stress study was performed to evaluate the range of polysorbate concentration able to maintain mAb stability against mechanical stress. The study evaluated Formulations containing PS20 concentrations within a range of 0.01-0.08% (w/v). Formulations containing 2 mg/mL and 40 mg/mL talquetamab in 15 mM acetate, 8% (w/v) sucrose, 0.02 mg/mL EDTA, pH 5.2 were spiked with PS20 to achieve 0.01, 0.02%, 0.04%, 0.06% and 0.08% (w/v) PS20 concentrations. The 2 mg/mL formulations were filled into 6 mL Type I glass vials with a 2.0 mL fill volume, and 40 mg/mL formulations were filled into 2 mL Type I glass vials with a 1.4 mL fill volume. The vials were placed on an orbital platform shaker and agitated at 250 rpm for 24 hours at ambient temperature. Samples were analyzed and the results are shown in Table 11 and Table 12.

For formulations varying in PS20 concentration from 0.02-0.08% (w/v), no meaningful changes were observed between formulations that were agitated for at least 24 hours. The Sub-Vis results for all PS20 concentrations and conditions, when viewed in the context of the magnitude of its compendial acceptance criteria values and overall methodology, can be considered essentially the same. There was a slight increase in HMWS (%) and loss of main component (%) for formulations containing 0.010 PS20 (Aggregate and Monomer %, respectively). However, these values are consistent with the definition of a stable aqueous pharmaceutical composition as provided for herein.

This study demonstrated that talquetamab formulations containing 0.01-0.08% (w/v) PS20 remained stable under mechanical stress conditions.

TABLE 11
Agitation Stress Study Results for 2 mg/mL and 40 mg/mL DP Formulations at
Varying Concentrations of PS20

| Formulation | | | | | | IE-HPLC | | |
|---|---|---|---|---|---|---|---|---|
| Surfactant | | | SE-HPLC | | | Acidic | Main | Basic |
| Concentration^{a)} | | | Monomer | Aggregate | Fragment | Peaks | Peak | Peaks |
| (w/v) | Condition | DP | (%) | (%) | (%) | (%) | (%) | (%) |
| 0.01% | | | 98.7 | 1.2 | <0.2 | 17.7 | 78.8 | 3.5 |
| 0.02% | | | 98.8 | 1.2 | <0.2 | 17.7 | 78.9 | 3.3 |
| 0.04% | T = 0 h | 2 mg/mL | 98.6 | 1.3 | <0.2 | 17.8 | 78.7 | 3.5 |
| 0.06% | | | 98.6 | 1.3 | <0.2 | 17.7 | 78.9 | 3.4 |
| 0.08% | | | 98.7 | 1.3 | <0.2 | 17.9 | 78.7 | 3.4 |
| 0.01% | | | 98.1 | 1.8 | <0.2 | 17.4 | 79 | 3.6 |
| 0.02% | | | 98.6 | 1.3 | <0.2 | 17.8 | 78.9 | 3.3 |
| 0.04% | T=24 h | 2 mg/mL | 98.7 | 1.3 | <0.2 | 17.8 | 78.8 | 3.3 |
| 0.06% | | | 98.7 | 1.3 | <0.2 | 17.5 | 79.1 | 3.4 |
| 0.08% | | | 98.7 | 1.3 | <0.2 | 17.6 | 78.9 | 3.5 |
| 0.01% | | | 98.5 | 1.5 | <0.2 | 17.7 | 78.9 | 3.4 |
| 0.02% | | | 98.5 | 1.5 | <0.2 | 17.8 | 78.8 | 3.4 |
| 0.04% | T = 0 h | 40 mg/mL | 98.7 | 1.3 | <0.2 | 17.7 | 79.1 | 3.3 |
| 0.06% | | | 98.5 | 1.5 | <0.2 | 17.9 | 78.6 | 3.5 |
| 0.08% | | | 98.5 | 1.5 | <0.2 | 17.9 | 78.7 | 3.5 |
| 0.01% | | | 97.6 | 2.4 | <0.2 | 17.2 | 78.6 | 4.2 |
| 0.02% | | | 98.5 | 1.5 | <0.2 | 17.5 | 78.9 | 3.6 |
| 0.04% | T = 24 h | 40 mg/mL | 98.6 | 1.4 | <0.2 | 17.4 | 79.3 | 3.3 |
| 0.06% | | | 98.5 | 1.5 | <0.2 | 17.8 | 78.7 | 3.4 |
| 0.08% | | | 98.4 | 1.6 | <0.2 | 17.9 | 78.5 | 3.5 |

^{a)}Formulation composition is 2 mg/mL or 40 mg/mL talquetamab in 15 mM acetate, 8% (w/v) sucrose, 0.02 mg/mL EDTA, pH 5.2 in addition to PS20 at levels described in the table.

TABLE 12
Agitation Stress Study Results for 2 mg/mL and 40 mg/mL DP Formulations at Varying
Concentrations of PS20

| | | | cSDS | | Sub-Visible | | |
|---|---|---|---|---|---|---|---|
| Formulation | | | (non- | cSDS | Particulate Matter | | |
| Surfactant | | | reduced) | (reduced) | ≥10 mm | ≥25 mm | |
| Concentration^{a)} | | | Purity | Purity | Particles/ | Particles/ | Potency |
| (w/v) | Condition | DP | (%) | (%) | Container | Container | (%) |
| 0.01% | | | 98.7 | 99.1 | 3 | 1 | NT |
| 0.02% | | | 98.6 | 99.2 | 2 | 0 | 106 |
| 0.04% | T = 0 h | 2 mg/mL | 98.6 | 99.1 | 8 | 2 | NT |
| 0.06% | | | 98.6 | 99.3 | 9 | 1 | 102 |
| 0.08% | | | 98.7 | 99 | 2 | 1 | NT |
| 0.01% | | | 98.5 | 99 | 15 | 1 | NT |
| 0.02% | | | 98.5 | 99.1 | 9 | 1 | 96 |
| 0.04% | T = 24 h | 2 mg/mL | 98.5 | 99.1 | 15 | 3 | NT |
| 0.06% | | | 98.4 | 99.1 | 10 | 0 | 99 |
| 0.08% | | | 98.6 | 99 | 13 | 1 | NT |
| 0.01% | | | 98.8 | 99 | 13 | 3 | NT |
| 0.02% | | | 98.7 | 99.1 | 7 | 1 | 101 |
| 0.04% | T = 0 h | 40 mg/mL | 98.7 | 99.2 | 68 | 1 | NT |
| 0.06% | | | 98.6 | 99.1 | 13 | 1 | 101 |
| 0.08% | | | 98.7 | 99.1 | 15 | 1 | NT |
| 0.01% | | | 98.6 | 99 | 10 | 2 | NT |
| 0.02% | | | 98.6 | 99.1 | 21 | 3 | 96 |
| 0.04% | T = 24 h | 40 mg/mL | 98.6 | 99 | 34 | 3 | NT |
| 0.06% | | | 98.5 | 99.1 | 18 | 3 | 102 |
| 0.08% | | | 98.5 | 98.9 | 22 | 5 | NT |

^{a)}Formulation composition is 2 mg/mL or 40 mg/mL talquetamab in 15 mM acetate, 8% (w/v) sucrose, 0.02 mg/mL EDTA, pH 5.2 in addition to PS20 at levels described in the table.

### Example 3: Freeze Thaw Stress Study

Studies were conducted to demonstrate the robustness of the formulation against physicochemical stresses induced by repeated freeze-thaw cycles.

In this study, 2 mg/mL and 40 mg/mL formulations (15 mM acetate, 8% (w/v) sucrose, 20 μg/mL EDTA, 0.04% (w/v) PS20, pH5.2) were filled into 5 mL size polycarbonate containers and placed upright in a −70° C. freezer. After freezing was complete, vials were removed from freezer and placed at ambient temperature for thawing. Once fully thawed, the containers were gently swirled to ensure solution homogeneity and then freezing thawing (F/T) was repeated until a total of 5 cycles were completed. Control samples did not undergo any freeze-thaw cycles. Both control and frozen and thawed samples were analyzed.

As presented in Table 13, quality attributes for samples that experienced five F/T cycles were nearly identical to the control samples that were stored at 2-8° C. and not subjected to the freeze-thaw conditions. Additionally, no significant changes in protein concentration, color of solution, pH, or turbidity were observed.

TABLE 13
Impact of repeated freeze-thaw cycles on 2
mg/mL and 40 mg/mL DP quality attributes

| | | 2 mg/mL | | 40 mg/mL | |
|---|---|---|---|---|---|
| Assay | Attributes | Control | 5X F/T | Control | 5X F/T |
| Particulate Matter | 10 μm Particles/container | 7 | 37 | 67 | 85 |
| (sub-vis) | | | | | |
| | 25 μm Particles/container | 1 | 0 | 1 | 1 |
| Bioassay | % Bioactivity | 91 | 84 | 97 | 106 |
| SE-HPLC | % Aggregate | 1.1 | 1.0 | 1.3 | 1.4 |
| | % Monomer | 98.9 | 98.9 | 98.7 | 98.6 |
| | % Fragment | <0.2 | <0.2 | <0.2 | <0.2 |
| IE-HPLC | % Total acidic species | 17.6 | 17.6 | 17.6 | 17.6 |
| | % Main peak | 79.1 | 79.2 | 79.2 | 78.9 |
| | % Total basic species | 3.3 | 3.2 | 3.2 | 3.4 |
| cSDS (non-reduced) | % Purity | 98.7 | 98.5 | 98.8 | 98.6 |
| cSDS (reduced) | % Purity | 99.1 | 99.1 | 99.1 | 99.1 |

The results of this study demonstrate the robustness of the formulation at 2 mg/mL and 40 mg/mL against freeze-thaw induced stress.

### Example 4: Metal Spiking Study

A study was performed to further evaluate the impact of metal ions potentially present or introduced in the Drug Product during manufacturing processes in DP formulation at 2 mg/mL and 40 mg/mL talquetamab.

Test formulations consisted of 2 mg/mL or 40 mg/mL talquetamab, 15 mM acetate, 8% sucrose, 20 μg/mL EDTA, and 0.04% PS 20 at pH 5.2 with or without the addition of Iron (Fe³⁺), Chromium (Cr³⁺), Copper (Cu²⁺), Nickel (Ni²⁺), and Molybdenum (Mo⁵)). The selection of metals is based on the composition of metal alloy components potentially present in manufacturing processes. To demonstrate robustness, exaggerated stress conditions were created by utilizing metal concentrations 5 to 13 times greater than values of trace metals historically reported in literature during DP manufacturing processes.

The 2 mg/mL test formulations were aliquoted into 6R vials at a fill volume of 3.5 mL, and 40 mg/mL test formulations were aliquoted into 2 mL vials at a fill volume of 2.0 mL. All vials were stoppered, capped, and crimp sealed. The vials were placed on stability at recommended (5° C.) and accelerated (25° C.) conditions for up to 6 months. At designated time points, samples were pulled and assayed.

As shown in Table 14, quality attributes for the 2 mg/mL and 40 mg/mL formulations stressed with metal ions after 6 months of storage at 2-8° C. were nearly identical to their respective control formulations. Further there was little to no change in quality attribute values of the 6 month compared to the T=0 values for all test formulations at stored at 2-8° C. Lastly, the quality attribute values of all metal spiked formulations and stored at 2-8° C. for six months were consistent with the definition of a stable aqueous pharmaceutical composition as provided for herein.

As shown in Table 15, quality attributes for the 2 mg/mL and 40 mg/mL formulations stressed with metal ions after 6 months of storage at 25° C. were nearly identical to their respective control formulations. A slight to minimal change in some quality attribute values was seen at 6 months as compared to the T=0 values for all test formulations. The degree of change was consistent with the expected degradation of those monoclonal antibodies quality attributes when at stored for six months at 25° C. Lastly, the quality attribute values of all metal spiked formulations and stored at 25° C. for six months were consistent with the definition of a stable aqueous pharmaceutical composition as provided for herein.

Taken together, the data demonstrate the robustness of the formulation at 2 mg/mL and 40 mg/mL against potential oxidative stresses experienced during normal and/or exaggerated Drug Product manufacturing processes and storage conditions.

TABLE 14
Impact of Metal Ions on 2 mg/mL and 40 mg/mL DP Stability for 6 months at
2-8° C.

| | | Formulation | | | | | | | |
|---|---|---|---|---|---|---|---|---|---|
| | | 2 mg/mL | | 2 mg/mL + metals | | 40 mg/mL | | 40 mg/mL + metals | |
| Assay | Attribute | T = 0 | 6 m | T = 0 | 6 m | T = 0 | 6 m | T = 0 | 6 m |
| Particulate | ≥10 μm | 16 | 7 | 18 | 3 | 6 | 23 | 3 | 2 |
| Matter | Particles/container | | | | | | | | |
| (sub-vis) | ≥25 μm | 2 | 1 | 2 | 0 | 1 | 1 | 0 | 0 |
| | Particles/container | | | | | | | | |
| Bioassay | % Bioactivity | 104 | 91 | 101 | 107 | 107 | 99 | 114 | 97 |
| SE-HPLC | % Aggregate | 0.9 | 0.8 | 0.9 | 0.8 | 1.2 | 1.3 | 1.2 | 1.4 |
| | % Monomer | 99.1 | 99.1 | 99.1 | 99.1 | 98.7 | 98.6 | 98.8 | 98.6 |
| | % Fragment | <0.2 | <0.2 | <0.2 | <0.2 | <0.2 | <0.2 | <0.2 | <0.2 |
| IE-HPLC | % Total acidic | 18.1 | 18.2 | 18.1 | 19 | 18.4 | 18.2 | 18.2 | 18.4 |
| | species | | | | | | | | |
| | % Main peak | 77.9 | 78.4 | 77.9 | 77.4 | 77.6 | 78.3 | 77.8 | 78.1 |
| | % Total basic | 4 | 3.3 | 4 | 3.6 | 4 | 3.5 | 4 | 3.6 |
| | species | | | | | | | | |
| Peptide | GPRC5D HC | 3.6 | 3.8 | 3.8 | 3.7 | 4 | 4.5 | 3.5 | 4.1 |
| Mapping | Met250/CD3 HC | | | | | | | | |
| | Met257 Oxidation | | | | | | | | |
| | (%) | | | | | | | | |
| | GPRC5D HC | 1.5 | 1.2 | 1.8 | 1.3 | 1.9 | 1.6 | 1.4 | 1.5 |
| | Met426/CD3 HC | | | | | | | | |
| | Met433 Oxidation | | | | | | | | |
| | (%) | | | | | | | | |
| cSDS (non- | % Purity | 98.8 | 97.6 | 98.6 | 98.6 | 98.6 | 98.5 | 98.4 | 98.5 |
| reduced) | | | | | | | | | |
| cSDS | % Purity | 99.1 | 99.1 | 99.1 | 99 | 98.9 | 99 | 99.1 | 99 |
| (reduced) | | | | | | | | | |

TABLE 15
Impact of Metal Ions on 2 mg/mL and 40 mg/mL DP Stability for 6 months at
25° C.

| | | Formulation | | | | | | | |
|---|---|---|---|---|---|---|---|---|---|
| | | 2 mg/mL | | 2 mg/mL + metals | | 40 mg/mL | | 40mg/mL + metals | |
| Assay | Attribute | T = 0 | 6 m | T = 0 | 6 m | T = 0 | 6 m | T = 0 | 6 m |
| Particulate | ≥10 mm | 16 | 8 | 18 | 7 | 6 | 39 | 3 | 7 |
| Matter | Particles/container | | | | | | | | |
| (sub-vis) | ≥25 mm | 2 | 1 | 2 | 1 | 1 | 2 | 0 | 0 |
| | Particles/container | | | | | | | | |
| Bioassay | % Bioactivity | 104 | 84 | 101 | 106 | 107 | 96 | 114 | 103 |
| SE-HPLC | % Aggregate | 0.9 | 0.8 | 0.9 | 1 | 1.2 | 1.8 | 1.2 | 1.8 |
| | % Monomer | 99.1 | 99.1 | 99.1 | 99 | 98.7 | 98.2 | 98.8 | 98.1 |
| | % Fragment | <0.2 | <0.2 | <0.2 | <0.2 | <0.2 | <0.2 | <0.2 | <0.2 |
| IE-HPLC | % Total acidic | 18.1 | 25.3 | 18.1 | 28 | 18.4 | 24.1 | 18.2 | 25 |
| | species | | | | | | | | |
| | % Main peak | 77.9 | 70.3 | 77.9 | 67 | 77.6 | 71.3 | 77.8 | 70.2 |
| | % Total basic | 4 | 4.4 | 4 | 5 | 4 | 4.6 | 4 | 4.8 |
| | species | | | | | | | | |
| Peptide | GPRC5D HC | 3.6 | 5.5 | 3.8 | 4.2 | 4 | 4.7 | 3.5 | 4.1 |
| Mapping | Met250/CD3 HC | | | | | | | | |
| | Met257 Oxidation | | | | | | | | |
| | (%) | | | | | | | | |
| | GPRC5D HC | 1.5 | 2 | 1.8 | 1.7 | 1.9 | 2 | 1.4 | 1.7 |
| | Met426/CD3 HC | | | | | | | | |
| | Met433 Oxidation | | | | | | | | |
| | (%) | | | | | | | | |
| cSDS (non- | % Purity | 98.8 | 98.1 | 98.6 | 98 | 98.6 | 97 | 98.4 | 97 |
| reduced) | | | | | | | | | |
| cSDS | % Purity | 99.1 | 97.7 | 99.1 | 97.4 | 98.9 | 98.2 | 99.1 | 98 |
| (reduced) | | | | | | | | | |

### Example 5: Formulation Robustness Development

### Study Design

A stable monoclonal antibody formulation is the result of the net effect of the complex interactions between the formulation's components. To evaluate these interactions, Design of Experiments (DOE) methodology was used to create a multi-factorial stability study in which a statistically determined number of experimental test formulations are generated that simultaneously vary the parameters of formulation components. Statistical analysis of the study results provides a quantitative understanding of how formulation parameters interact to impact stability attributes and, in turn, demonstrate the robustness of formulation.

Experimental test formulations of GPRC5D×CD3 drug product were held at recommended (5° C.) for up to 12 months and accelerated (25° C.) conditions for up 6 months. The formulation components evaluated were protein concentration, acetate concentration, sucrose concentration, polysorbate 20 concentration, EDTA concentration, and pH. The ranges of the factor concentrations tested are listed in Table 16 (target formulation values included in table to provide context). The study encompassed both 2 mg/mL and 40 mg/mL DP formulations. A typical protein concentration range for evaluation is +/−12% of the target value. Therefore, the upper protein concentration values selected for the study represent 112% of the of the 40 mg/mL formulations. For convenience, 1 mg/mL was used the lower protein concentration (50% of the 2 mg/mL formulation).

TABLE 16
Ranges of the factor concentrations tested

| Test Factors | Low | Target | High |
|---|---|---|---|
| Protein (mg/mL) | 1 | n/a | 45 |
| pH | 4.6 | 5.2 | 5.8 |
| Acetate (mM) | 10 | 15 | 20 |
| Sucrose concentration (% w/v) | 6 | 8 | 10 |
| EDTA concentration (μg/mL) | 7.5 | 20 | 32.5 |
| PS20 concentration (% w/v) | 0.015 | 0.04 | 0.065 |

Based on this criterion, JMP® statistical software was used to create a 6-factor, resolution IV, Fractional Factorial Design (Table 17).

TABLE 17
Compositions of Study Test Formulations

| Study Test | Talquetamab | | Acetate | Sucrose | EDTA | PS20 |
|---|---|---|---|---|---|---|
| Formulation | (mg/mL) | pH | (mM) | (% w/v) | (μg/mL) | (% w/v) |
| 1 | 45 | 4.7 | 10 | 10 | 32.5 | 0.015 |
| 2 | 1 | 5.7 | 10 | 10 | 7.5 | 0.065 |
| 3 | 1 | 5.7 | 20 | 10 | 32.5 | 0.015 |
| 4 | 1 | 5.7 | 10 | 6 | 32.5 | 0.015 |
| 5 | 1 | 4.7 | 20 | 6 | 32.5 | 0.065 |
| 6 | 45 | 5.7 | 10 | 10 | 7.5 | 0.015 |
| 7 | 45 | 4.7 | 20 | 10 | 7.5 | 0.065 |
| 8 | 45 | 5.7 | 10 | 6 | 32.5 | 0.065 |
| 9 | 1 | 4.7 | 10 | 10 | 32.5 | 0.065 |
| 10 | 45 | 4.7 | 10 | 6 | 7.5 | 0.065 |
| 11 | 1 | 4.7 | 10 | 6 | 7.5 | 0.015 |
| 12 | 1 | 5.7 | 20 | 6 | 7.5 | 0.065 |
| 13 | 45 | 4.7 | 20 | 6 | 32.5 | 0.015 |
| 14 | 45 | 5.7 | 20 | 10 | 32.5 | 0.065 |
| 15 | 45 | 5.7 | 20 | 6 | 7.5 | 0.015 |
| 16 | 1 | 4.7 | 20 | 10 | 7.5 | 0.015 |

The vials were stoppered, capped, and crimp sealed. The vials were placed on stability at recommended (5° C.) and accelerated (25° C.) conditions. At designated time points, samples were pulled and assayed.

### Study Results

The test results for each attribute of the sixteen formulations at study initiation (time zero), after 6 months at accelerated temperature (25° C.), and after six and 12 months at the recommended storage condition (5° C.) are presented in Table 18. The data are reported as the range, mean, and standard deviation of the sixteen formulations for each attribute.

The analytical results for all formulations held for 12 months at 5° C. demonstrated little change in the assay test values, indicating stability. The ability for all formulations with multi-variant ranges in excipient concentrations to yield a narrow range of assay test result values demonstrated the robustness of the formulation within the boundaries and storage conditions tested. Additionally, the full range of values observed in this study were consistent with the definition of a stable aqueous pharmaceutical composition as provided for herein when held for 12 months at 5° C.

The analytical results for all formulations held for 6 months at accelerated (25° C.) storage conditions showed degradation effects consistent with the stability profile of GPRC5D×CD3 antibody exposed to prolonged accelerated storage conditions as demonstrated by the increase in the magnitude of the range of result values as compared to both study initiation (time zero) and 12 months at 5° C. However, the calculated average value and the full range of values observed for all of the nine assay results were consistent with the definition of a stable aqueous pharmaceutical composition as provided for herein when held for 6 months at accelerated (25° C.) storage conditions.

TABLE 18
Summary of Study Data (Range, Mean, and Standard Deviation)

| | | Time and Storage Conditions | | | | | | | | |
|---|---|---|---|---|---|---|---|---|---|---|
| | | T = 0M | | | 6M 25° C. | | | 12M 5° C. | | |
| Assay | | Range | Mean | SD | Range | Mean | SD | Range | Mean | SD |
| cIEF | % area Main | 76.1-78.6 | 78.1 | 0.7 | 59.0-72.6 | 68.1 | 3.9 | 76.3-77.8 | 77.3 | 0.4 |
| | Peak | | | | | | | | | |
| | % Sum of | 17.6-18.6 | 18.0 | 0.3 | 21.7-36.5 | 26.4 | 4.5 | 18.1-20.2 | 18.8 | 0.6 |
| | Acidic peaks | | | | | | | | | |
| | % Sum of | 3.1-6.3 | 3.9 | 1.0 | 4.3-8.9 | 5.6 | 1.5 | 3.5-4.4 | 3.9 | 0.3 |
| | Basic peaks | | | | | | | | | |
| cSDS | % Purity | 98.1-98.6 | 98.4 | 0.1 | 96.2-98.1 | 97.1 | 0.5 | 97.8-97.8 | 98.3 | 0.2 |
| | (non-reduced) | | | | | | | | | |
| | % Purity | 98.7-99.2 | 99.0 | 0.2 | 96.6-98.4 | 97.6 | 0.6 | 98.6-98.9 | 98.8 | 0.1 |
| | (reduced) | | | | | | | | | |
| SE- | % Aggregate | 0.6-1.6 | 1.0 | 0.3 | 0.5-2.6 | 1.4 | 0.8 | 0.5-2.1 | 1.2 | 0.6 |
| HPLC | | | | | | | | | | |
| | % Monomer | 98.4-99.4 | 99.0 | 0.3 | 97.3-99.5 | 98.6 | 0.8 | 97.9-99.5 | 98.8 | 0.6 |
| | % Fragment | N/A | 0.0 | 16 of 16 | 0.0-0.1 | 0.1 | 10 of 16 | ND-<0.2 | N/A | 14 of 16 |
| | | | | <0.1 | | | <0.1 | | | <0.2 |

### Statistical Analysis of Study Results

A factorial regression model was used to perform statistical analysis of the 12M 2-8° C. data to determine which factors had a statistically significant effect verse a given response. Table 19 listed the calculated p-values for each factor verses a given response and corresponding adjusted R-squared values generated by the model. p-values less than 0.05 are considered statistically significant.

TABLE 19
calculated p-values for each factor verses a given response

| | p-Value | | | | | | |
|---|---|---|---|---|---|---|---|
| | cIEF | | | cSDS | | SEC | |
| | Acid | Basic | Main | Reduced | Non- | Aggregate | Monomer |
| Term | (%) | (%) | (%) | (%) | Reduced (%) | (%) | (%) |
| API | 0.073 | 0.874 | 0.008 | 0.522 | 0.592 | 0.000 | 0.000 |
| Sucrose | 0.817 | 0.874 | 0.684 | 0.522 | 0.747 | 0.815 | 0.707 |
| Acetate | 1.000 | 1.000 | 0.544 | 0.418 | 0.343 | 0.815 | 0.534 |
| pH | 0.003 | 0.063 | 0.001 | 0.610 | 0.253 | 0.001 | 0.001 |
| PS20 | 0.729 | 0.751 | 0.544 | 0.309 | 0.747 | 1.000 | 0.900 |
| EDTA | 0.492 | 1.000 | 0.176 | 0.841 | 0.914 | 0.815 | 0.534 |
| R-sq(adj) | 50.40% | 0.00% | 66.50% | 0.00% | 0.00% | 87.81% | 89.50% |

### API Concentration

API concentration showed a statistically significant negative correlation with % monomer while also showing a corresponding, statistically significant positive correlation with % aggregate. These trends indicate that increased API concentration correlates with decreased stability as measured by SEC. However, within the concentration range evaluated in this study (1-90 mg/mL), the range of % monomer and % aggregate assay results observed were consistent with the definition of a stable aqueous pharmaceutical composition as provided for herein.

API concentration also showed a statistically significant positive correlation with % main peak. However, as shown in Table 19, the range of result values for % main peak is considered extremely narrow for this attribute. Therefore, while calculated to be of statistical significance, API concentration from 1-90 mg/mL is not considered to have a practical impact on the % main peak.

### Formulation pH

Formulation pH showed a statistically significant negative correlation with % monomer while also showing a corresponding, statistically significant positive correlation with % aggregate. These trends indicate that increased pH correlates with decreased stability as measured by SEC. However, within the pH range evaluated in this study (4.7-5.7), the range of % monomer and % aggregate assay results observed were consistent with the definition of a stable aqueous pharmaceutical composition as provided for herein.

pH also showed a statistically significant negative correlation with % main peak while also showing a corresponding, statistically significant positive correlation with % acid peak. However, similar to API concentration, and as shown in Table 19, the range of result values for % acid peak and % main peak considered extremely narrow for those three attributes. Therefore, while calculated to be of statistical significance, pH from 4.7 to 5.7 is not considered to have a practical impact on the % acid and % main peak.

### Example 6: Formulated Drug Bulk Production

### Process Description

### Processing Solutions

TABLE 20
Processing Solutions Target Composition and Ranges

| Solution | Composition and Ranges |
|---|---|
| Diafiltration Buffer | 10 mM Acetate, 8.6% Sucrose (w/v), pH 4.9 |
| Polysorbate 20, EDTA | 10 mM Acetate, 8% (w/v) Sucrose; 4% (w/v) PS20; 2 g/L EDTA, pH |
| Stock Solution | 5.2 |

### Ultrafiltration/Diafiltration (UF/DF)

Ultrafiltration/diafiltration (UF/DF) was performed to re-formulate the talquetamab Planova filtrate intermediate manufacturing solution to a pre-formulated bulk (pFB) solution consisting of 90 mg/mL talquetamab, 10 mM Acetate, 8% (w/v) Sucrose, pH 5.2.

### Preparation of Talquetamab Formulated Bulk (FB)

Polysorbate 20 (4.0% w/v) and EDTA (2 mg/mL) stock solution was added to the pFB at a 1:100 dilution to obtain a final concentration of 0.04% (w/v) Polysorbate 20, and 20 μg/mL EDTA yielding the Formulated Bulk (FB) consisting of 90 mg/mL talquetamab in 15 mM Acetate, 8% (w/v) Sucrose, 0.04% Polysorbate 20, 20 μg/mL EDTA, pH 5.2. The FB solution was then mixed uniformly. Final filtration of the Formulated Bulk was achieved using a sterile 0.45/0.22 μm filter immediately followed with a subsequent, in-line 0.22 μm filter.

### Final Bulk Fill

Following final filtration, the FB was filled into polycarbonate Biotainer(s). The fill volume is 20% to 90% of the biotainer's stated volume.

### Final Bulk Storage and Shipping

Storage and Shipment Conditions of the Formulated Bulk Prior to Drug Product production was 5° C.±3° C. protected from light if FB was stored for about one week or less or −40° C.±10° C. protected from light if FB was stored for more than one week.

### Example 7: 40 mg/mL DP Solution Production

### Dilution of Talquetamab Formulated Bulk (FB)

Dilution was performed to re-formulate the talquetamab Formulated Bulk intermediate manufacturing solution to Drug Product solution consisting of 40 mg/mL talquetamab, 15 mM Acetate, 8% (w/v) Sucrose, 20 μg/mL, EDTA, pH 5.2

### Dilution Buffer

Provided herein is a tabular summary of the composition of the Dilution Buffer in Table 21. Final filtration of the dilution buffer was achieved using a sterile 0.22 μm filter.

TABLE 21
Composition of Dilution Buffer

| Component | Composition | | Amount per mL | |
|---|---|---|---|---|
| Sodium acetate trihydrate | 15 | mM | 1.497 | mg |
| Glacial acetic acid | | | 0.240 | mg |
| Sucrose | 8% (w/v) | | 80 | mg |
| Polysorbate 20 | 0.04% | | 0.40 | mg |
| EDTA Disodium salt, Dihydrate | 20 | μg/mL | 0.02 | mg |

| | | |
|---|---|---|
| Water for Injection | q.s to 1.0 mL | q.s to 1.0 mL |

Preparation of Talquetamab 40 mg/mL Drug Product Solution

Dilution Buffer was added to the 90 mg/mL FB yielding a 40 mg/mL Drug Product solution consisting of 40 mg/mL talquetamab in 15 mM Acetate, 8% (w/v) Sucrose, 0.04% Polysorbate 20, 20 μg/mL EDTA, pH 5.2.

### Example 8: 40 mg/mL Drug Product Formulation: Composition and Components of Primary Packaging

Provided herein are tabular summaries of compositions of the 40 mg/mL Talquetamab Drug Product Formulation, in Table 22 and Table 23.

TABLE 22
Composition of 40 mg/mL Talquetamab Drug Product

| Component | Amount per mL | |
|---|---|---|
| Talquetamab | 40 | mg |
| Sodium acetate trihydrate | 1.497 | mg |
| Glacial acetic acid | 0.240 | mg |
| Sucrose | 80 | mg |
| Polysorbate 20 | 0.40 | mg |
| EDTA Disodium salt, Dihydrate | 0.02 | mg |

| | |
|---|---|
| Water for Injection | q.s to 1.0 mL |

TABLE 23
Composition of 40 mg/mL Talquetamab Drug Product

| Component | Amount per mL | |
|---|---|---|
| Talquetamab | 40 | mg |
| Sodium acetate | 0.90 | mg |
| Glacial acetic acid | 0.240 | mg |
| Sucrose | 80 | mg |
| Polysorbate 20 | 0.40 | mg |
| Component | Amount per mL | |
| EDTA Disodium | 0.018 | mg |

| | |
|---|---|
| Water for Injection | q.s to 1.0 mL |

The 40 mg/mL Talquetamab drug product (DP) primary packaging consists of a glass vial, a polymer vial stopper, and an aluminum seal. Table 24 provides the specific components for the primary packaging material of the 40 mg/mL DP presentation.

TABLE 24
40 mg/mL DP Primary packaging material components

| Component | Description |
|---|---|
| Glass vial | 2 mL glass Type 1 borosilicate |
| Stopper | 13 mm Stopper 4432/50 Gray with Flurotec Coating, RTS |
| Seals | 13 mm aluminum seal with flip-off cap |

### Example 9: 2 mg/mL DP Solution Production

### Dilution of Talquetamab Formulated Bulk (FB)

Dilution was performed to re-formulate the talquetamab Formulated Bulk intermediate manufacturing solution to Drug Product solution consisting of 2 mg/mL talquetamab, 15 mM Acetate, 8% (w/v) Sucrose, 20 μg/mL, EDTA, pH 5.2

### Dilution Buffer

Provided herein is a tabular summary of the composition of the Dilution Buffer in Table 25. Final filtration of the dilution buffer was achieved using a sterile 0.22 μm filter.

TABLE 25
Composition of Dilution Buffer

| Component | Composition | | Amount per mL | |
|---|---|---|---|---|
| Sodium acetate trihydrate | 15 | mM | 1.497 | mg |
| Glacial acetic acid | | | 0.240 | mg |
| Sucrose | 8% (w/v) | | 80 | mg |
| Polysorbate 20 | 0.04% | | 0.40 | mg |
| EDTA Disodium salt, Dihydrate | 20 | μg/mL | 0.02 | mg |

| | | |
|---|---|---|
| Water for Injection | q.s to 1.0 mL | q.s to 1.0 mL |

Preparation of Talquetamab 2 mg/mL Drug Product Solution

Dilution Buffer was added to the 90 mg/mL FB yielding a 2 mg/mL Drug Product solution consisting of 2 mg/mL talquetamab in 15 mM Acetate, 8% (w/v) Sucrose, 0.04% Polysorbate 20, 20 μg/mL EDTA, pH 5.2.

### Example 10: 2 mg/mL Drug Product Formulation: Composition and Components of Primary Packaging

Provided herein are tabular summaries of compositions of the 2 mg/mL Talquetamab Drug Product Formulation, in Table 26 and Table 27.

TABLE 26
Composition of 2 mg/mL Talquetamab Drug Product

| Component | Amount per mL | |
|---|---|---|
| Talquetamab | 2 | mg |
| Sodium acetate trihydrate | 1.497 | mg |
| Glacial acetic acid | 0.240 | mg |
| Sucrose | 80 | mg |
| Polysorbate 20 | 0.40 | mg |
| EDTA Disodium salt, Dihydrate | 0.02 | mg |

| | |
|---|---|
| Water for Injection | q.s to 1.0 mL |

TABLE 27
Composition of 2 mg/mL Talquetamab Drug Product

| Component | Amount per 1.5 mL | |
|---|---|---|
| Talquetamab | 3 | mg |
| Sodium acetate | 1.39 | mg |
| Glacial acetic acid | 0.36 | mg |
| Sucrose | 120 | mg |
| Polysorbate 20 | 0.60 | mg |
| EDTA Disodium | 0.027 | mg |

| | |
|---|---|
| Water for Injection | q.s to 1.5 mL |

The 2 mg/mL talquetamab drug product (DP) primary packaging consists of a glass vial, a polymer vial stopper, and an aluminum seal. Table 28 lists specific components for the primary packaging material of the 2 mg/mL DP presentation.

TABLE 28
2 mg/mL DP Primary packaging material components

| Component | Description |
|---|---|
| Glass vial | 6R glass Type 1 borosilicate |
| Stopper | 20 mm Stopper 4023/50 Gray with Flurotec Coating, RTS |
| Seals | 20 mm aluminum seal with flip-off cap |

### Example 11: Description of 40 mg/mL Stability Study

This study was conducted to monitor talquetamab 40 mg mg/mL Drug Product attributes placed on stability under various environmental conditions and lengths of time. Study test articles were prepared by aliquoting Formulated Bulk into 2 mL vials at a fill volume of 1.4 mL. The vials were stoppered, capped, and crimp sealed

All studies were to be performed with vials in an inverted orientation.

TABLE 29
Study parameters

| Stability Classification | Storage condition | Duration (Months) |
|---|---|---|
| Real-time | 5 ± 3° C. | 36 |
| Accelerated | 25 ± 2° C./60% RH | 12 |
| Stressed | 40 ± 2° C./75% RH | 6 |

### Stability Study Results

The stability results for talquetamab DP held under recommended, accelerated, and stressed conditions are listed below. At all-time points for DP held at recommended storage conditions (about 12 months or more and at a temperature of about 5° C., and/or after storage for about 2 years or more and at a temperature of about 5° C.), all test parameter result values observed per assay study exceeded the criteria consistent with the definition of a stable aqueous pharmaceutical composition as provided for herein. Similarly, peptide map results showed little to no consequential change over time in the measured percent of post translational modification.

Results for talquetamab DP held at accelerated and stressed conditions showed the expected rates of degradation for Drug Product exposed to prolonged accelerated and stressed storage conditions. Of particular note, all test parameter result values observed per assay study for DP held at accelerated conditions (25° C.) for 12 months showed results consistent with the definition of a stable aqueous pharmaceutical composition as provided for herein.

TABLE 30
Stability Results for 40 mg/mL Talquetamab Drug Product Stored at 5° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | Color of | | Turbidity | particles | particles | Purity: | |
| Months | Solution | pH | (NTU) | per vial | per vial | % | new peaks |
| 0 | ≤BY6, ≤B7, | 5.1 | 4.7 | 53 | 1 | 99.1 | No new peak > 1.0% |
| | ≤Y6 | | | | | | compared to Reference |
| | | | | | | | Material |
| 3 | ≤BY7, ≤B8, | 5.3 | 4.9 | 17 | 1 | 99.1 | No new peak > 1.0% |
| | ≤Y7 | | | | | | compared to Reference |
| | | | | | | | Material |
| 6 | ≤BY7, ≤B7, | 5.3 | 4.8 | 184 | 13 | 98.8 | No new peak > 1.0% |
| | ≤Y6 | | | | | | compared to Reference |
| | | | | | | | Material |
| 9 | ≤BY6, ≤B7, | 5.2 | 4.9 | 208 | 11 | 98.9 | No new peak > 1.0% |
| | ≤Y6 | | | | | | compared to Reference |
| | | | | | | | Material |
| 12 | ≤B7, ≤BY8, | 5.3 | 4.9 | 27 | 1 | 98.9 | No new peak > 1.0% |
| | ≤Y7 | | | | | | compared to Reference |
| | | | | | | | Material |

TABLE 31
Stability Results for 40 mg/mL Talquetamab Drug Product Stored at 5° C.
(Continued)

| | | | | | | | Potency T-Cell |
|---|---|---|---|---|---|---|---|
| | | | SE HPLC | | | | Activation |
| | cSDS (Non-reduced) | | Main | | | Protein Conc. | Assay |
| | Purity | New Peaks | Component | HMWS | LMWS | by A₂₈₀ | % of RM |
| Months | (%) | (%) | (%) | (%) | (%) | (mg/mL) | activity |
| 0 | 98.6 | No new peak > | 98.7 | 1.3 | <0.2 | 40.4 | 97 |
| | | 1.0% compared | | | | | |
| | | to Reference | | | | | |
| | | Material | | | | | |
| 3 | 98.5 | No new peak > | 98.6 | 1.4 | <0.2 | 40.5 | 92 |
| | | 1.0% compared | | | | | |
| | | to Reference | | | | | |
| | | Material | | | | | |
| 6 | 98.6 | No new peak > | 98.6 | 1.4 | <0.2 | 40.5 | 93 |
| | | 1.0% compared | | | | | |
| | | to Reference | | | | | |
| | | Material | | | | | |
| 9 | 98.5 | No new peak > | 98.7 | 1.3 | <0.2 | 40.6 | 94 |
| | | 1.0% compared | | | | | |
| | | to Reference | | | | | |
| | | Material | | | | | |
| 12 | 98.4 | No new peak > | 98.6 | 1.4 | <0.2 | 40.7 | 90 |
| | | 1.0% compared | | | | | |
| | | to Reference | | | | | |
| | | Material | | | | | |

TABLE 32
Stability Results for 40 mg/mL Talquetamab
Drug Product Stored at 5° C. (Continued)

| | IE-HPLC | | | Polysorbate |
|---|---|---|---|---|
| | Sum of acidic | Main | Sum of Basic | 20 |
| Months | peaks (%) | peak (%) | peaks (%) | (%) |
| 0 | 19.1 | 77.4 | 3.5 | 0.035 |
| 3 | 19 | 77.5 | 3.5 | 0.038 |
| 6 | 19.1 | 77.3 | 3.6 | 0.036 |
| 9 | 19.4 | 77 | 3.7 | 0.036 |
| 12 | 19.4 | 76.8 | 3.7 | 0.038 |

TABLE 33
Stability Results for 40 mg/mL Talquetamab
Drug Product Stored at 5° C. (Continued)

| | Post Translational Modification | | |
|---|---|---|---|
| | GPRC5D HC | GPRC5D HC | CD3 HC |
| | Met 250/CD3 HC | Met 426/CD3 HC | Asn103/106 |
| | Met 257 Oxidation | Met433 Oxidation | Deamidation |
| Months | (%) | (%) | (%) |
| 0 | 2.9 | 1.1 | 13 |
| 3 | 3.1 | 1.1 | 12.3 |
| 6 | 3.5 | 1.3 | 13.1 |
| 9 | 3.3 | 1.1 | 14.8 |

### 25° C. Data

TABLE 34
Stability Results for 40 mg/mL Talquetamab Drug Product Stored at 25° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | Color of | | Turbidity | particles | particles | Purity: | |
| Months | Solution | pH | (NTU) | per vial | per vial | % | new peaks |
| 0 | ≤BY6, ≤B7, | 5.1 | 4.7 | 53 | 1 | 99.1 | No new peak > 1.0% |
| | ≤Y6 | | | | | | compared to Reference |
| | | | | | | | Material |
| 3 | ≤BY7, ≤B8, | 5.3 | 5.3 | 74 | 3 | 98.8 | No new peak > 1.0% |
| | ≤Y6 | | | | | | compared to Reference |
| | | | | | | | Material |
| 6 | ≤BY7, ≤B8, | 5.3 | 5.1 | 27 | 1 | 98.4 | No new peak > 1.0% |
| | ≤Y7 | | | | | | compared to Reference |
| | | | | | | | Material |
| 9 | ≤BY5, ≤B6, | 5.2 | 6 | 41 | 1 | 98.1 | No new peak > 1.0% |
| | ≤Y6 | | | | | | compared to Reference |
| | | | | | | | Material |

TABLE 35
Stability Results for 40 mg/mL Talquetamab Drug Product Stored at 25° C.
(Continued)

| | | | | | | | Potency T-Cell |
|---|---|---|---|---|---|---|---|
| | | | SE HPLC | | | | Activation |
| | cSDS (Non-reduced) | | Main | | | Protein Conc. | Assay |
| | Purity | New Peaks | Component | HMWS | LMWS | by A₂₈₀ | % of RM |
| Months | (%) | (%) | (%) | (%) | (%) | (mg/mL) | activity |
| 0 | 98.6 | No new peak > | 98.7 | 1.3 | <0.2 | 40.4 | 97 |
| | | 1.0% compared | | | | | |
| | | to Reference | | | | | |
| | | Material | | | | | |
| 3 | 97.7 | No new peak > | 98.4 | 1.6 | <0.2 | 40.5 | 107 |
| | | 1.0% compared | | | | | |
| | | to Reference | | | | | |
| | | Material | | | | | |
| 6 | 97.8 | No new peak > | 98.2 | 1.7 | <0.2 | 40.7 | 95 |
| | | 1.0% compared | | | | | |
| | | to Reference | | | | | |
| | | Material | | | | | |
| 9 | 97.1 | No new peak > | 98.3 | 1.7 | <0.2 | 40.7 | 82 |
| | | 1.0% compared | | | | | |
| | | to Reference | | | | | |
| | | Material | | | | | |

TABLE 36
Stability Results for 40 mg/mL Talquetamab
Drug Product Stored at 25° C. (Continued)

| | IE-HPLC | | | Polysorbate |
|---|---|---|---|---|
| | Sum of acidic | Main | Sum of Basic | 20 |
| Months | peaks (%) | peak (%) | peaks (%) | (%) |
| 0 | 19.1 | 77.4 | 3.5 | 0.0349 |
| 3 | 21.7 | 74.4 | 4 | 0.038 |
| 6 | 25.1 | 70 | 4.9 | 0.033 |
| 9 | 27.4 | 67.7 | 4.9 | 0.037 |

TABLE 37
Stability Results for 40 mg/mL Talquetamab
Drug Product Stored at 25° C. (Continued)

| | Post Translational Modification | | |
|---|---|---|---|
| | GPRC5D HC | GPRC5D HC | CD3 HC |
| | Met 250/CD3 HC | Met 426/CD3 HC | Asn103/106 |
| | Met 257 Oxidation | Met433 Oxidation | Deamidation |
| Months | (%) | (%) | (%) |
| 0 | 2.9 | 1.1 | 13 |
| 3 | 3.7 | 1.2 | 14.8 |
| 6 | 4.7 | 1.9 | 17.8 |
| 9 | 4.6 | 1.3 | 18.8 |

### 40° C. Data

TABLE 38
Stability Results for 40 mg/mL Talquetamab Drug Product Stored at 40° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | Color of | | Turbidity | particles | particles | Purity: | |
| Months | Solution | pH | (NTU) | per vial | per vial | % | new peaks |
| 0 | ≤BY6, ≤B7, | 5.1 | 4.7 | 53 | 1 | 99.1 | No new peak > 1.0% |
| | ≤Y6 | | | | | | compared to Reference |
| | | | | | | | Material |
| 1 | ≤B6, ≤BY7, | 5.2 | 5.4 | 170 | 10 | 98.2 | No new peak > 1.0% |
| | ≤Y6 | | | | | | compared to Reference |
| | | | | | | | Material |
| 3 | ≤B7, ≤BY7, | 5.3 | 5.6 | 191 | 10 | 96.9 | No new peak > 1.0% |
| | ≤Y6 | | | | | | compared to Reference |
| | | | | | | | Material |
| 6 | ≤B6, <BY5, | 5.3 | 6.5 | 228 | 9 | 89.2 | New peak 2.60% CMT |
| | ≤Y6 | | | | | | 15.5 |

TABLE 39
Stability Results for 40 mg/mL Talquetamab Drug Product Stored at 40° C.
(Continued)

| | | | | | | | Potency T-Cell |
|---|---|---|---|---|---|---|---|
| | | | SE HPLC | | | | Activation |
| | cSDS (Non-reduced) | | Main | | | Protein Conc. | Assay |
| | Purity | New Peaks | Component | HMWS | LMWS | by A₂₈₀ | % of RM |
| Months | (%) | (%) | (%) | (%) | (%) | (mg/mL) | activity |
| 0 | 98.6 | No new peak > | 98.7 | 1.3 | <0.2 | 40.4 | 97 |
| | | 1.0% compared | | | | | |
| | | to Reference | | | | | |
| | | Material | | | | | |
| 1 | 97 | No new peak > | 98 | 1.9 | <0.2 | 40.7 | 80 |
| | | 1.0% compared | | | | | |
| | | to Reference | | | | | |
| | | Material | | | | | |
| 3 | 93.4 | No new peak > | 96.5 | 3.1 | 0.4 | 40 | 65 |
| | | 1.0% compared | | | | | |
| | | to Reference | | | | | |
| | | Material | | | | | |
| 6 | 89 | New peak 1.32% | 93.5 | 5.9 | 0.5 | 40.5 | 35 |
| | | CMT 25.2 | | | | | |

TABLE 40
Stability Results for 40 mg/mL Talquetamab
Drug Product Stored at 40° C. (Continued)

| | IE-HPLC | | | Polysorbate |
|---|---|---|---|---|
| | Sum of acidic | Main | Sum of Basic | 20 |
| Months | peaks (%) | Peaks (%) | peaks (%) | (%) |
| 0 | 19.1 | 77.4 | 3.5 | 0.0349 |
| 1 | 27.9 | 66.6 | 5.5 | 0.036 |
| 3 | 39.4 | 53.6 | 7 | 0.034 |
| 6 | 56.9 | 31.9 | 11.2 | 0.0275 |

TABLE 41
Stability Results for 40 mg/mL Talquetamab
Drug Product Stored at 40° C. (Continued)

| | Post Translational Modification | | |
|---|---|---|---|
| | GPRC5D HC Met | GPRC5D HC | CD3 HC |
| | 250/ CD3 HC | Met 426/CD3 | Asn103/106 |
| | Met 257 Oxidation | HC Met433 Oxidation | Deamidation |
| Months | (%) | (%) | (%) |
| 0 | 2.9 | 1.1 | 13 |
| 1 | 4 | 1.3 | 19.1 |
| 3 | 6.2 | 2 | 30 |
| 6 | 12.4 | 4.5 | 46.1 |

### Example 12: Description of 2 mg/mL Stability Study

This study was conducted to monitor talquetamab 2 mg/mL Drug Product attributes placed on stability under various environmental conditions and lengths of time. Study test articles were prepared by aliquoting Formulated Bulk into 6R vials at a fill volume of 2.0 mL. The vials were stoppered, capped, and crimp sealed.

All studies were to be performed with vials in an inverted orientation.

TABLE 42
Study parameters

| Stability Classification | Storage condition | Duration (Months) |
|---|---|---|
| Real-time | 5 ± 3° C. | 36 |
| Accelerated | 25 ± 2° C./60% RH | 12 |
| Stressed | 40 ± 2° C./75% RH | 6 |

### Stability Study Results

The stability results for talquetamab DP held under recommended, accelerated, and stressed conditions are listed below. At all-time points for DP held at recommended storage conditions (about 12 months or more and at a temperature of about 5° C., and/or after storage for about 2 years or more and at a temperature of about 5° C.), all test parameter result values observed per assay study exceeded the criteria consistent with the definition of a stable aqueous pharmaceutical composition as provided for herein. Similarly, peptide map results showed little to no consequential change over time in the measured percent of post translational modification.

Results for talquetamab DP held at accelerated and stressed conditions showed the expected rates of degradation for Drug Product exposed to prolonged accelerated and stressed storage conditions. Of particular note, the majority of test parameter result values observed per assay study for DP held at accelerated conditions (25° C.) for 12 months showed results consistent with the definition of a stable aqueous pharmaceutical composition as provided for herein, with all but one of the results falling within the defined stability parameters. Interestingly, the sole outlier was at a six month timepoint, wherein the nine month data indicate that the DP is stable out to nine months at accelerated conditions. This may indicate that the 6 month data for T-Cell activation represent an outlier and as such the DP may be considered to be consistent with the definition of a stable aqueous pharmaceutical composition as provided for herein for all test parameter results.

### 5° C. Data

TABLE 43
Stability Results for 2 mg/mL Talquetamab Drug Product Stored at 5° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | Color of | | Turbidity | particles | particles | Purity: | |
| Months | Solution | pH | (NTU) | per vial | per vial | % | new peaks |
| 0 | ≤B7, ≤BY9, | 5.1 | 1 | 412 | 37 | 99.1 | No new peak > 1.0% |
| | ≤Y7 | | | | | | compared to Reference |
| | | | | | | | Material |
| 3 | ≤B7, ≤BY9, | 5.2 | 0.9 | 499 | 33 | 99 | No new peak > 1.0% |
| | ≤Y7 | | | | | | compared to Reference |
| | | | | | | | Material |
| 6 | ≤B7, ≤BY9, | 5.2 | 1.1 | 69 | 0 | 99 | No new peak > 1.0% |
| | ≤Y7 | | | | | | compared to Reference |
| | | | | | | | Material |
| 9 | ≤B7, ≤BY9, | 5.2 | 1 | 749 | 60 | 98.8 | No new peak > 1.0% |
| | ≤Y7 | | | | | | compared to Reference |
| | | | | | | | Material |
| 12 | ≤B7, ≤BY9, | 5.2 | 0.9 | 24 | 0 | 99 | No new peak > 1.0% |
| | ≤Y7 | | | | | | compared to Reference |
| | | | | | | | Material |

TABLE 44
Stability Results for 2 mg/mL Talquetamab Drug Product Stored at 5° C. (Continued)

| | | | | | | | Potency T-Cell |
|---|---|---|---|---|---|---|---|
| | | | SE HPLC | | | | Activation |
| | cSDS (Non-reduced) | | Main | | | Protein Conc. | Assay |
| | Purity | New Peaks | Component | HMWS | LMWS | by A₂₈₀ | % of RM |
| Months | (%) | (%) | (%) | (%) | (%) | (mg/mL) | activity |
| 0 | 98.5 | No new peak > | 99.2 | 0.8 | <0.2 | 2 | 91 |
| | | 1.0% compared | | | | | |
| | | to Reference | | | | | |
| | | Material | | | | | |
| 3 | 98.5 | No new peak > | 99.1 | 0.9 | <0.2 | 2.1 | 96 |
| | | 1.0% compared | | | | | |
| | | to Reference | | | | | |
| | | Material | | | | | |
| 6 | 97.9 | No new peak > | 99.2 | 0.8 | <0.2 | 2 | 83 |
| | | 1.0% compared | | | | | |
| | | to Reference | | | | | |
| | | Material | | | | | |
| 9 | 98.3 | No new peak > | 99.2 | 0.8 | <0.2 | 2.1 | 107 |
| | | 1.0% compared | | | | | |
| | | to Reference | | | | | |
| | | Material | | | | | |
| 12 | 98.4 | No new peak > | 99.1 | 0.9 | <0.2 | 2.1 | 99 |
| | | 1.0% compared | | | | | |
| | | to Reference | | | | | |
| | | Material | | | | | |

TABLE 45
Stability Results for 2 mg/mL Talquetamab
Drug Product Stored at 5° C. (Continued)

| | IE-HPLC | | | Polysorbate |
|---|---|---|---|---|
| | Sum of acidic | Main | Sum of Basic | 20 |
| Months | peaks (%) | peak (%) | peaks (%) | (%) |
| 0 | 19.2 | 77.5 | 3.3 | 0.039 |
| 3 | 19.1 | 77.6 | 3.3 | 0.04 |
| 6 | 19.3 | 77.2 | 3.6 | 0.038 |
| 9 | 19.7 | 76.7 | 3.6 | 0.037 |
| 12 | 19.7 | 76.7 | 3.6 | 0.04 |

TABLE 46
Stability Results for 2 mg/mL Talquetamab
Drug Product Stored at 5° C. (Continued)

| | Post Translational Modification | | |
|---|---|---|---|
| | GPRC5D HC | GPRC5D HC | CD3 HC |
| | Met 250/CD3 HC | Met 426/CD3 HC | Asn103/106 |
| | Met 257 Oxidation | Met433 Oxidation | Deamidation |
| Months | (%) | (%) | (%) |
| 0 | 0.8 | 4 | 13.4 |
| 9 | 0.4 | 4.2 | |

### 25° C. Data

TABLE 47
Stability Results for 2 mg/mL Talquetamab Drug Product Stored at 25° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | Color of | | Turbidity | particles | particles | Purity: | |
| Months | Solution | pH | (NTU) | per vial | per vial | % | new peaks |
| 0 | ≤B9, ≤BY7, | 5.1 | 1 | 412 | 37 | 99.1 | No new peak > 1.0% |
| | <Y7 | | | | | | compared to Reference |
| | | | | | | | Material |
| 3 | ≤B9, ≤BY7, | 5.2 | 0.9 | 44 | 2 | 98.6 | No new peak > 1.0% |
| | ≤Y7 | | | | | | compared to Reference |
| | | | | | | | Material |
| 6 | ≤B8, ≤BY7, | 5.1 | 1 | 38 | 0 | 97.4 | No new peak > 1.0% |
| | ≤Y7 | | | | | | compared to Reference |
| | | | | | | | Material |
| 9 | ≤B6, ≤BY5, | 5.2 | 1 | 12 | 0 | 96.3 | No new peak > 1.0% |
| | ≤Y6 | | | | | | compared to Reference |
| | | | | | | | Material |

TABLE 48
Stability Results for 2 mg/mL Talquetamab Drug Product Stored at 25° C. (Continued)

| | | | | | | | Potency T-Cell |
|---|---|---|---|---|---|---|---|
| | | | SE HPLC | | | | Activation |
| | cSDS (Non-reduced) | | Main | | | Protein Conc. | Assay |
| | Purity | New Peaks | Component | HMWS | LMWS | by A₂₈₀ | % of RM |
| Months | (%) | (%) | (%) | (%) | (%) | (mg/mL) | activity |
| 0 | 98.5 | No new peak > | 99.2 | 0.8 | <0.2 | 2 | 91 |
| | | 1.0% compared | | | | | |
| | | to Reference | | | | | |
| | | Material | | | | | |
| 3 | 97.8 | No new peak > | 99 | 1 | <0.2 | 2 | 91 |
| | | 1.0% compared | | | | | |
| | | to Reference | | | | | |
| | | Material | | | | | |
| 6 | 97.5 | No new peak > | 99 | 1 | <0.2 | 2.1 | 22 |
| | | 1.0% compared | | | | | |
| | | to Reference | | | | | |
| | | Material | | | | | |
| 9 | 97.1 | No new peak > | 99.1 | 0.9 | <0.2 | 2 | 81 |
| | | 1.0% compared | | | | | |
| | | to Reference | | | | | |
| | | Material | | | | | |

TABLE 49
Stability Results for 2 mg/mL Talquetamab Drug Product Stored at 25° C.
(Continued)

| | IE-HPLC | | | |
|---|---|---|---|---|
| | Sum of acidic | Main peak | Sum of Basic | Polysorbate 20 |
| Months | peaks (%) | (%) | peaks (%) | (%) |
| 0 | 19.2 | 77.5 | 3.3 | 0.039 |
| 3 | 22.8 | 73.4 | 3.9 | 0.038 |
| 6 | 26.3 | 67.4 | 6.3 | 0.036 |
| 9 | 31.3 | 64 | 4.7 | 0.038 |

TABLE 50
Stability Results for 2 mg/mL Talquetamab Drug Product Stored at 25° C.
(Continued)

| | Post Translational Modification | | |
|---|---|---|---|
| | GPRC5D HC Met | GPRC5D HC Met | CD3 HC |
| | 250/CD3 HC Met 257 | 426/CD3 HC Met433 | Asn103/106 |
| | Oxidation | Oxidation | Deamidation |
| Months | (%) | (%) | (%) |
| 0 | 0.8 | 4 | 13.4 |
| 6 | 2.8 | 6.6 | 19 |
| 9 | 0.3 | 7 | 22.3 |

### 40° C. Data

TABLE 51
Stability Results for 2 mg/mL Talquetamab Drug Product Stored at 40° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | Color of | | Turbidity | particles | particles | Purity: | |
| Months | Solution | pH | (NTU) | per vial | per vial | % | new peaks |
| 0 | ≤B9, | 5.1 | 1 | 412 | 37 | 99.1 | No new peak > 1.0% |
| | ≤BY7, ≤Y7 | | | | | | compared to Reference |
| | | | | | | | Material |
| 1 | ≤B9, | 5.2 | 1 | 55 | 1 | 97.3 | No new peak > 1.0% |
| | ≤BY7, ≤Y7 | | | | | | compared to Reference |
| | | | | | | | Material |
| 3 | ≤B8, | 5.2 | 1.1 | 47 | 0 | 94.1 | New peak 1.87% CMT 15.86 |
| | ≤BY7, ≤Y7 | | | | | | |
| 6 | ≤B8, | 5.3 | 1.2 | 63 | 0 | 82.8 | New peak: Peak2 3.18% |
| | ≤BY7, ≤Y6 | | | | | | CMT 15.8, Peak 3 3.10% |
| | | | | | | | CMT 15.5 |

TABLE 52
Stability Results for 2 mg/mL Talquetamab Drug Product Stored at 40° C. (Continued)

| | | | | | | | Potency T-Cell |
|---|---|---|---|---|---|---|---|
| | | | SE HPLC | | | | Activation |
| | cSDS (Non-reduced) | | Main | | | Protein Conc. | Assay |
| | Purity | New Peaks | Component | HMWS | LMWS | by A₂₈₀ | % of RM |
| Months | (%) | (%) | (%) | (%) | (%) | (mg/mL) | activity |
| 0 | 98.5 | No new peak > | 99.2 | 0.8 | <0.2 | 2 | 91 |
| | | 1.0% compared | | | | | |
| | | to Reference | | | | | |
| | | Material | | | | | |
| 1 | 97.2 | No new peak > | 99 | 1 | <0.2 | 2 | 84 |
| | | 1.0% compared | | | | | |
| | | to Reference | | | | | |
| | | Material | | | | | |
| 3 | 93.3 | No new peak > | 98 | 1.8 | 0.2 | 2 | 59 |
| | | 1.0% compared | | | | | |
| | | to Reference | | | | | |
| | | Material | | | | | |
| 6 | 89 | New peak 3 | 94.9 | 4.7 | 0.5 | 2 | 88 |
| | | 1.38% CMT 25.3 | | | | | |

TABLE 53
Stability Results for 2 mg/mL Talquetamab
Drug Product Stored at 40° C. (Continued)

| | IE-HPLC | | | Polysorbate |
|---|---|---|---|---|
| | Sum of acidic | Main | Sum of Basic | 20 |
| Months | peaks (%) | peak (%) | peaks (%) | (%) |
| 0 | 19.2 | 77.5 | 3.3 | 0.039 |
| 1 | 29 | 66 | 5 | 0.038 |
| 3 | 49.3 | 43.6 | 7.1 | 0.037 |
| 6 | 71.6 | 20.2 | 71.6 | 0.0328 |

TABLE 54
Stability Results for 2 mg/mL Talquetamab
Drug Product Stored at 40° C. (Continued)

| | Post Translational Modification | | |
|---|---|---|---|
| | GPRC5D HC | GPRC5D HC | CD3 HC |
| | Met 250/CD3 HC | Met 426/CD3 HC | Asn103/106 |
| | Met 257 Oxidation | Met433 Oxidation | Deamidation |
| Months | (%) | (%) | (%) |
| 0 | 0.8 | 4 | 13.4 |
| 1 | 0.8 | 4.8 | 19.9 |
| 6 | 20.9 | 7.7 | 60.6 |

### Example 13: Stability of Formulations Containing 30 mg/mL, 60 mg/mL, and 90 mg/mL Talquetamab

To examine the stability of formulations with higher talquetamab concentrations, three formulations that contained 30 mg/mL, 60 mg/mL, and 90 mg/mL talquetamab respectively, were placed on stability at 2-8° C. and 25° C. for up to 18 months. Target composition was 15 mM acetate, 8% (w/v) sucrose, 20 μg/mL EDTA disodium, 0.04% (w/v) PS 20 at pH 5.2 for all three formulations. The formulations were analyzed for main component, aggregate and fragment content by SE-HPLC, purity by cSDS (reduced and non-reduced), charge heterogeneity by clEF, and sub-visible particulate matter by light obscuration (LO).

No substantial changes were observed for clEF and cSDS (reduced and non-reduced) data as well as sub-visible particle counts (data not shown).

The SE-HPLC data (Table 55) of all three formulations show a nearly identical stability profile over 18 months at 2-8° C. with all values reported consistent with the most preferred embodiment of stability.

Comparing the stability profiles of all three formulations over 3 months at 25° C. shows a slight but present correlation of increased protein concentration and decreased main component. However, the magnitude of the decrease occurred over a narrow range with all protein concentrations showing main component values consistent with the most preferred embodiment of stability.

TABLE 55
SE-HPLC Data for 30, 60, and 90 mg/mL Formulations

| | Main component (%) | | | | | |
|---|---|---|---|---|---|---|
| Timepoint | 30 mg/mL | | 60 mg/mL | | 90 mg/mL | |
| (months) | 2-8° C. | 25° C. | 2-8° C. | 25° C. | 2-8° C. | 25° C. |
| T₀ | 98.9 | 98.9 | 98.6 | 98.6 | 98.6 | 98.6 |
| 1 | 98.9 | 98.7 | 98.6 | 98.3 | 98.5 | 98.1 |
| 3 | 98.8 | 98.5 | 98.5 | 98.1 | 98.3 | 97.9 |
| 8ª | 98.6 | NA | 98.4 | NA | 98.2 | NA |
| 12 | 98.5 | NA | 98.4 | NA | 98.2 | NA |
| 18 | 98.4 | NA | 98.3 | NA | 98.1 | NA |

^{a}30 mg/mL formulation sample pull occurred at 6 month timepoint
NA = Not applicable

### Example 14: Phase 1 Study of Talquetamab Administered as Monotherapy for Relapsed or Refractory Multiple Myeloma

The efficacy of talquetamab monotherapy was evaluated in patients with relapsed or refractory multiple myeloma in a single-arm, open-label, multicenter study, MMY1001 (MonumenTAL-1) (NCT03399799, NCT04634552). The study included patients who had previously received at least three prior therapies, including a proteasome inhibitor, an immunomodulatory agent, and an anti-CD38 monoclonal antibody. The study included patients who received prior T cell redirection therapy (N=34). Patients received talquetamab 0.4 mg/kg subcutaneously weekly, following two step-up doses (0.01 and 0.06 mg/kg) in the first week of therapy, or talquetamab 0.8 mg/kg subcutaneously biweekly (every 2 weeks), following three step-up doses (0.01, 0.06 and 0.3 mg/kg), until disease progression or unacceptable toxicity. Patients were hospitalized for monitoring for at least 48 hours after each talquetamab dose during the step-up phase.

A significant number of patients in the subcutaneous cohorts received the formulation shown in Table 27 for the 2 mg/mL concentration of talquetamab (for step-up doses of 0.01 mg/kg and 0.06 mg/kg) and the formulation shown in Table 23 for the 40 mg/mL concentration of talquetamab (for doses of 0.4 mg/kg and 0.8 mg/kg).

Of 122 patients treated with talquetamab 0.4 mg/kg weekly who were not exposed to prior T cell redirection therapy, the median age was 67 (range: 47 to 86) years, 53% were male, 92% were White, and 7% were Black or African American. Patients had received a median of 5 (range: 3 to 13) prior therapies, and 77% of patients had received prior autologous stem cell transplantation (ASCT). Ninety-three percent (93%) of patients were refractory to their last therapy, and 75% were refractory to a proteasome inhibitor, immunomodulatory agent, and anti-CD38 antibody. Of the 113 patients for whom baseline cytogenetic data were available, high-risk cytogenetic factors (presence of t(4:14), t(14:16), and/or del(17p)) were present in 35% of patients.

Of 109 patients treated with talquetamab 0.8 mg/kg biweekly (every 2 weeks) who were not exposed to prior T cell redirection therapy, the median age was 67 (range: 38 to 82) years, 61% were male, 87% were White, and 6% were Black or African American. Patients had received a median of 5 (range: 3 to 12) prior therapies, and 79% of patients had received prior autologous stem cell transplantation (ASCT). Ninety-five percent (95%) of patients were refractory to their last therapy and 69% were refractory to a proteasome inhibitor (PI), immunomodulatory agent, and anti-CD38 antibody. Of the 94 patients for whom baseline cytogenetic data were available, high-risk cytogenetic factors (presence of t(4:14), t(14:16), and/or del(17p)) were present in 33% of patients.

Efficacy results were based on overall response rate (ORR) assessed by an Independent Review Committee using IMWG criteria.

TABLE 56
Efficacy Results for MMY1001 (MonumenTAL-1) in
Patients Receiving 0.4 mg/kg Weekly talquetamab

| | 0.4 mg/kg Weekly |
|---|---|
| | (N = 122) |
| Overall response rate (ORR = sCR + CR + VGPR + PR) | 91 (74.6%) |
| 95% CI (%) | (65.9, 82.0) |
| Stringent complete response (sCR) | 24% |
| Complete response (CR) | 10% |
| Very good partial response (VGPR) | 25% |
| Partial response (PR) | 16% |

Duration of Response (DOR)

| | |
|---|---|
| Number of responders | 91 |
| Median DOR (95% CI) (months) | 8.8 (6.5, 12.5) |
| Patients with DOR ≥ 6 months | 66% |
| Patients with DOR ≥ 12 months | 42% |

MRD Negativity Rate^{a}

| | |
|---|---|
| MRD negativity rate in all treated patients, n (%) | 40 (32.8%) |
| 95% CI (%) | (24.6, 41.9) |
| MRD negativity rate^{b} in patients achieving CR or sCR | |
| Number of patients with CR or better | N = 41 |
| MRD negativity rate, n (%) | 20 (48.8%) |
| 95% CI (%) | (32.9, 64.9) |

CI = confidence interval;
MRD = minimal residual disease;
NE = not estimable
Median duration of follow-up was 14.7 months.
^{a}MRD-negativity rate is defined as the proportion of participants who achieved MRD negative status (at 10-5) at any timepoint after initial dose and prior to progressive disease (PD) or subsequent anti-myeloma therapy.
^{b}Only MRD assessments (10-5 testing threshold) within 3 months of achieving CR/sCR until death/progression/subsequent therapy (exclusive) are considered.

TABLE 57
Efficacy Results for MMY1001 (MonumenTAL-1) in Patients
Receiving 0.8 mg/kg Biweekly (Every 2 Weeks) talquetamab

| | 0.8 mg/kg Biweekly |
|---|---|
| | (Every 2 Weeks) |
| | (N = 109) |
| Overall response rate (ORR = sCR + CR + VGPR + PR) | 81 (74.3%) |
| 95% CI (%) | (65.1, 82.2) |
| Stringent complete response (sCR) | 18% |
| Complete response (CR) | 14% |
| Very good partial response (VGPR) | 26% |
| Partial response (PR) | 17% |

Duration of Response (DOR)

| | |
|---|---|
| Number of responders | 81 |
| Patients with DOR ≥ 6 months | 86% |
| Patients with DOR ≥ 9 months | 82% |

MRD Negativity Rate^{a}

| | |
|---|---|
| MRD negativity rate in all treated patients, n (%) | 31 (28.4%) |
| 95% CI (%) | (20.2, 37.9) |
| MRD negativity rate^{b} in patients achieving CR or | |
| sCR | |
| Number of patients with CR or better | N = 35 |
| MRD negativity rate, n (%) | 11 (31.4%) |
| 95% CI (%) | (16.9, 49.3) |

CI = confidence interval;
MRD = minimal residual disease;
NE = not estimable
Median duration of follow-up = 8.1 months.
^{a}MRD-negativity rate is defined as the proportion of participants who achieved MRD negative status (at 105) at any timepoint after initial dose and prior to progressive disease (PD) or subsequent anti-myeloma therapy.
^{b}Only MRD assessments (10-5 testing threshold) within 3 months of achieving CR/sCR until death/progression/subsequent therapy (exclusive) are considered.

The median time to first response was 1.2 (range: 0.2 to 10.9) months and 1.3 (range: 0.2 to 9.2) months for 0.4 mg/kg weekly and 0.8 mg/kg biweekly (every 2 weeks), respectively. ORR results were consistent across pre-specified subgroups, including number of prior lines of therapy, refractoriness to prior therapy, and cytogenetic risk at baseline.

MMY1001 also included 34 patients who were exposed to prior T-cell redirection therapy and had received at least three prior therapies, including a PI, an immunomodulatory agent, and an anti-CD38 monoclonal antibody. Patients received talquetamab 0.4 mg/kg subcutaneously once a week, following 2 step-up doses (0.01 and 0.06 mg/kg) until disease progression or unacceptable toxicity. The median age was 60 (range: 38 to 78) years, 59% were male, 97% were White, and 3% were Black or African American. Patients had received a median of 6 (range: 3 to 15) prior therapies. Prior T-cell redirection therapy was CAR-T cell therapy for 82% of patients and bispecific antibody treatment for 24%. With a median duration of follow-up of 9.9 months, ORR per IRC assessment was 71%.

Talquetamab exposure was comparable at the two dosing schedules. No clinically significant effect of anti-talquetamab antibodies on PK, efficacy, or AEs was observed. PD changes were comparable at both dosing schedules and consistent with talquetamab activity, including T-cell activation, redistribution, and induction of cytokines. In conclusion, talquetamab demonstrated robust efficacy and manageable safety in patients with heavily pretreated relapsed or refractory multiple myeloma.

1. An aqueous pharmaceutical composition comprising:
a) a concentration of about 0.5 mg/mL to about 3.5 mg/mL of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
(1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
b) about 10 mM to about 20 mM of acetate and/or pharmaceutically acceptable acetate salt;
c) about 6% (w/v) to about 10% (w/v) of sucrose;
d) about 7 μg/mL to about 33 μg/mL of ethylenediaminetetraacetic acid (EDTA);
e) about 0.01% to about 0.07% polysorbate 20; and
f) a pH from about 4.6 to about 5.8.

2. An aqueous pharmaceutical composition comprising:
a) a concentration of about 25 mg/mL to about 55 mg/mL of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
(1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
b) about 10 mM to about 20 mM of acetate and/or pharmaceutically acceptable acetate salt;
c) about 6% (w/v) to about 10% (w/v) of sucrose;
d) about 7 μg/mL to about 33 μg/mL of ethylenediaminetetraacetic acid (EDTA);
e) about 0.01% to about 0.07% polysorbate 20; and
f) a pH from about 4.6 to about 5.8.

3. The aqueous pharmaceutical composition of claim 1, wherein the bispecific GPRC5D/CD3 antibody comprises a VH1 having the amino acid sequence of SEQ ID NO:7, and a VL1 having the amino acid sequence of SEQ ID NO:8.

4. The aqueous pharmaceutical composition of claim 1, wherein the bispecific GPRC5D/CD3 antibody comprising a HC1 having the amino acid sequence of SEQ ID NO:9, and a LC1 having the amino acid sequence of SEQ ID NO:10.

5. The aqueous pharmaceutical composition of claim 1, wherein the bispecific GPRC5D/CD3 antibody comprises a VH2 having the amino acid sequence of SEQ ID NO:17, and a VL2 having the amino acid sequence of SEQ ID NO:18.

6. The aqueous pharmaceutical composition of claim 1, wherein the bispecific GPRC5D/CD3 antibody comprises a HC2 having the amino acid sequence of SEQ ID NO:19, and a LC2 having the amino acid sequence of SEQ ID NO:20.

7. The aqueous pharmaceutical composition of claim 1, wherein the bispecific GPRC5D/CD3 antibody is talquetamab.

8. The aqueous pharmaceutical composition of claim 1, wherein the bispecific GPRC5D/CD3 antibody has a concentration of about 1 mg/mL to about 3 mg/mL.

9. The aqueous pharmaceutical composition of claim 8, wherein the bispecific GPRC5D/CD3 antibody has a concentration of about 1.5 mg/mL to about 2.5 mg/mL.

10. The aqueous pharmaceutical composition of claim 9, wherein the bispecific GPRC5D/CD3 antibody has a concentration of about 2 mg/mL.

11. The aqueous pharmaceutical composition of claim 2, wherein the bispecific GPRC5D/CD3 antibody has a concentration of about 30 mg/mL to about 50 mg/mL.

12. The aqueous pharmaceutical composition of claim 11, wherein the bispecific GPRC5D/CD3 antibody has a concentration of about 35 mg/mL to about 45 mg/mL.

13. The aqueous pharmaceutical composition of claim 12, wherein the bispecific GPRC5D/CD3 antibody has a concentration of about 40 mg/mL.

14. The aqueous pharmaceutical composition of claim 1, wherein the composition comprises about 12 mM to about 18 mM of acetate and/or pharmaceutically acceptable acetate salt.

15. The aqueous pharmaceutical composition of claim 1, wherein the composition comprises about 14 mM to about 16 mM of acetate and/or pharmaceutically acceptable acetate salt.

16. The aqueous pharmaceutical composition of claim 1, wherein the composition comprises about 15 mM of acetate and/or pharmaceutically acceptable acetate salt.

17. The aqueous pharmaceutical composition of claim 1, wherein the composition comprises about 7% (w/v) to about 9% (w/v) of sucrose.

18. The aqueous pharmaceutical composition of claim 17, wherein the composition comprises about 8% (w/v) of sucrose.

19. The aqueous pharmaceutical composition of claim 1, wherein the composition comprises about 15 μg/mL to about 21 μg/mL of EDTA.

20. The aqueous pharmaceutical composition of claim 1, wherein the composition comprises about 18 μg/mL of EDTA.

21. The aqueous pharmaceutical composition of claim 1, wherein the composition comprises about 0.02% to about 0.06% of PS-20.

22. The aqueous pharmaceutical composition of claim 21, wherein the composition comprises about 0.03 to about 0.05% of PS-20.

23. The aqueous pharmaceutical composition of claim 22, wherein the composition comprises about 0.04% of PS-20.

24. The aqueous pharmaceutical composition of claim 1, wherein the pH is about 4.8 to about 5.6.

25. The aqueous pharmaceutical composition of claim 24, wherein the pH is about 4.9 to about 5.5.

26. The aqueous pharmaceutical composition of claim 25, wherein the pH is about 5.2.

27. The aqueous pharmaceutical composition of claim 1, wherein the composition comprises 2 mg/mL of the bispecific GPRC5D/CD3 antibody, 15 mM of acetate and/or pharmaceutically acceptable acetate salt, 8% (w/v) sucrose, 18 μg/mL of EDTA, 0.04% PS 20, and a pH of 5.2.

28. The aqueous pharmaceutical composition of claim 2, wherein the composition comprises 40 mg/mL of the bispecific GPRC5D/CD3 antibody, 15 mM of acetate and/or pharmaceutically acceptable acetate salt, 8% (w/v) sucrose, 18 μg/mL of EDTA, 0.04% PS 20, and a pH of 5.2.

29. The aqueous pharmaceutical composition of claim 1, wherein the aqueous pharmaceutical composition is stable.

30. The aqueous pharmaceutical composition of claim 29, wherein stability is defined based on color of solution, pH, turbidity, percentage of purity, percentage of new peaks, percentage of main component, percentage of high molecular weight species (HWMS), percentage of low molecular weight species (LMWS), percentage of sum of acidic peaks, percentage of sum of basic peaks, protein concentration, percentage of T cell activation, percentage of PS 20 (w/v), or any combination thereof.

31. The aqueous pharmaceutical composition of claim 29, wherein the aqueous pharmaceutical composition is stable at a temperature of about 2-8° C. for at least two years.

32. A method of treating cancer in a subject in need thereof, the method comprising administering to the subject the aqueous pharmaceutical composition of claim 1.

33. The method of claim 32, wherein the administering is subcutaneous.

34. A method for preparing an aqueous pharmaceutical composition of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
(1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
the method comprising combining a composition comprising about 2 mg/mL of the bispecific GPRC5D/CD3 antibody, about 15 mM of acetate and/or a pharmaceutically acceptable acetate salt, about 8% (w/v) sucrose, about 18 μg/mL of EDTA, and about 0.04% polysorbate (PS) 20, wherein the stable aqueous pharmaceutical composition has a pH of about 5.2.

35. A method for preparing an aqueous pharmaceutical composition of a bispecific G-protein coupled receptor, class C, group 5, member D (GPRC5D)/cluster of differentiation 3 (CD3) antibody or antigen-binding fragment thereof, the bispecific GPRC5D/CD3 antibody or antigen-binding fragment thereof comprising:
(1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs: 1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs: 4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs: 11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs: 14, 15, and 16, respectively;
the method comprising combining a composition comprising about 40 mg/mL of the bispecific GPRC5D/CD3 antibody, about 15 mM of acetate and/or a pharmaceutically acceptable acetate salt, about 8% (w/v) sucrose, about 18 μg/mL of EDTA, and about 0.04% polysorbate (PS) 20, wherein the stable aqueous pharmaceutical composition has a pH of about 5.2.

36. The method of claim 34, wherein the bispecific GPRC5D/CD3 antibody comprises a VH1 having the amino acid sequence of SEQ ID NO:7, and a VL1 having the amino acid sequence of SEQ ID NO:8.

37. The method of claim 34, wherein the bispecific GPRC5D/CD3 antibody comprising a HC1 having the amino acid sequence of SEQ ID NO:9, and a LC1 having the amino acid sequence of SEQ ID NO:10.

38. The method of claim 34, wherein the bispecific GPRC5D/CD3 antibody comprises a VH2 having the amino acid sequence of SEQ ID NO:17, and a VL2 having the amino acid sequence of SEQ ID NO:18.

39. The method of claim 34, wherein the bispecific GPRC5D/CD3 antibody comprises a HC2 having the amino acid sequence of SEQ ID NO:19, and a LC2 having the amino acid sequence of SEQ ID NO:20.

40. The method of claim 34, wherein the bispecific GPRC5D/CD3 antibody is talquetamab.

41. The method of claim 34, wherein the aqueous pharmaceutical composition is stable.

42. The method of claim 41, wherein stability is defined based on color of solution, pH, turbidity, percentage of purity, percentage of new peaks, percentage of main component, percentage of high molecular weight species (HWMS), percentage of low molecular weight species (LMWS), percentage of sum of acidic peaks, percentage of sum of basic peaks, protein concentration, percentage of T cell activation, percentage of PS 20 (w/v), or any combination thereof.

43. A kit comprising the aqueous pharmaceutical composition of claim 1 and instructions for use.

44. An article of manufacture comprising a container holding the aqueous pharmaceutical composition of claim 1.

45. The article of manufacture of claim 44, wherein the container is a vial with a stopper pierceable by a syringe.

46. The aqueous pharmaceutical composition of claim 1 for use in the treatment of cancer.

47. The aqueous pharmaceutical composition of claim 1 for use in the preparation of a medicament for the treatment of cancer.

48. Use of the aqueous pharmaceutical composition of claim 1 for treating a cancer in a subject in need thereof, the use comprising administering the aqueous pharmaceutical composition to the subject in need thereof.

49. The use of claim 48, wherein the administration is subcutaneous.


SEQUENCE LISTING (39 sequences)

SEQ ID NO: 1 - protein, 5 aa, synthetic construct
GYTMN

SEQ ID NO: 2 - protein, 17 aa, synthetic construct
LINPYNSDTNYAQKLQG

SEQ ID NO: 3 - protein, 9 aa, synthetic construct
VALRVALDY

SEQ ID NO: 4 - protein, 11 aa, synthetic construct
KASQNVATHVG

SEQ ID NO: 5 - protein, 7 aa, synthetic construct
SASYRYS

SEQ ID NO: 6 - protein, 9 aa, synthetic construct
QQYNRYPYT

SEQ ID NO: 7 - protein, 118 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 8 - protein, 107 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 9 - protein, 445 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
TYAMN

SEQ ID NO: 12 - protein, 19 aa, synthetic construct
RIRSKYNNYATYYAASVKG

SEQ ID NO: 13 - protein, 14 aa, synthetic construct
HGNFGNSYVSWFAY

SEQ ID NO: 14 - protein, 14 aa, synthetic construct
RSSTGAVTTSNYAN

SEQ ID NO: 15 - protein, 7 aa, synthetic construct
GTNKRAP

SEQ ID NO: 16 - protein, 9 aa, synthetic construct
ALWYSNLWV

SEQ ID NO: 17 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 18 - protein, 112 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 19 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 20 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 345 aa, Homo sapiens
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 22 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 23 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 24 - protein, 7 aa, synthetic construct
GYSFTGY

SEQ ID NO: 25 - protein, 6 aa, synthetic construct
NPYNSD

SEQ ID NO: 26 - protein, 7 aa, synthetic construct
GFTFNTY

SEQ ID NO: 27 - protein, 8 aa, synthetic construct
RSKYNNYA

SEQ ID NO: 28 - protein, 10 aa, synthetic construct
GYSFTGYTMN

SEQ ID NO: 29 - protein, 10 aa, synthetic construct
LINPYNSDTN

SEQ ID NO: 30 - protein, 10 aa, synthetic construct
GFTFNTYAMN

SEQ ID NO: 31 - protein, 12 aa, synthetic construct
RIRSKYNNYATY

SEQ ID NO: 32 - protein, 8 aa, synthetic construct
GYSFTGYT

SEQ ID NO: 33 - protein, 8 aa, synthetic construct
INPYNSDT

SEQ ID NO: 34 - protein, 11 aa, synthetic construct
ARVALRVALDY

SEQ ID NO: 35 - protein, 6 aa, synthetic construct
QNVATH

SEQ ID NO: 36 - protein, 8 aa, synthetic construct
GFTFNTYA

SEQ ID NO: 37 - protein, 10 aa, synthetic construct
IRSKYNNYAT

SEQ ID NO: 38 - protein, 16 aa, synthetic construct
ARHGNFGNSYVSWFAY

SEQ ID NO: 39 - protein, 9 aa, synthetic construct
TGAVTTSNY